(19) United States
(12) Patent Application Publication
(10) Pub. No.: US 2019/0282131 A1
(43) Pub. Date: Sep. 19, 2019

(54) MANAGEMENT OF BIOMECHANICAL ACHIEVEMENTS

(71) Applicant: SEISMIC HOLDINGS, INC., Menlo Park, CA (US)

(72) Inventors: Andrew Robert Chang, Sunnyvale, CA (US); Derek Chang, Menlo Park, CA (US); Daniel Le Ly, Mountain View, CA (US); Ray Franklin Cowan, Mountain View, CA (US)

(21) Appl. No.: 16/355,340

(22) Filed: Mar. 15, 2019

Related U.S. Application Data
(60) Provisional application No. 62/643,640, filed on Mar. 15, 2018.
(60) Provisional application No. 62/646,814, filed on Mar. 22, 2018.
(60) Provisional application No. 62/813,387, filed on Mar. 4, 2019.

(51) Int. Cl. A61B 5/11; A63B 24/00; A61H 3/00; A41D 1/00; G09B 19/00; A61B 5/00

(52) U.S. Cl. CPC A61B 5/1123; A61B 2503/20; A61H 3/00; A41D 1/002; G09B 19/003; A61B 5/1112; A61B 5/7267; A63B 2220/12; A63B 2220/803; A63B 2220/51; A63B 2220/54; A63B 2230/60; A61H 2201/165; A63B 2230/04; A63B 2024/0096; A61B 2505/09; A63B 24/0062

(57) ABSTRACT

Systems and methods and media for managing biomechanical achievements are provided. An exosuit or any other suitable sensor assembly worn by a user can be utilized by a system to monitor several movement factors that may characterize the user's movement and any changes in the user's movement with a high degree of specificity that may enable various system algorithms and/or models to predict or otherwise determine one or more biomechanical achievements of the user, such as recovery from a particular type of event (e.g., surgery or therapy procedure) and/or distance traveled (e.g., without using any global positioning system capabilities). In addition, an exosuit can provide useful feedback in response to such determinations.

### CROSS-REFERENCE TO RELATED APPLICATION(S)

This application claims priority to U.S. Provisional Patent Application No. 62/643,640, filed Mar. 15, 2018, U.S. Provisional Patent Application No. 62/646,814, filed Mar. 22, 2018, and U.S. Provisional Patent Application No. 62/813,387, filed Mar. 4, 2019, each of which is incorporated by reference herein in its entirety.

### TECHNICAL FIELD

This disclosure relates generally to the field of biomechanical achievements, and more specifically to systems and methods and media for determining a user's biomechanical achievements for providing an effective and efficient user experience.

### BACKGROUND

Wearable robotic systems have been developed for augmentation of humans' natural capabilities or to replace functionality lost due to injury or illness. It may be desirable to monitor wearers of such systems for determining certain biomechanical achievements.

### SUMMARY

Systems, methods, and media for determining a user's biomechanical achievements are discussed herein.

For example, a method for managing biomechanical achievements using a biomechanical model custodian system is provided that may include receiving, at the biomechanical model custodian system, first experiencing entity data including first biomechanical movement data indicative of a first type of biomechanical movement made by a first experiencing entity prior to experiencing a first procedure on at least one anatomical feature of the first experiencing entity and second biomechanical movement data indicative of the first type of biomechanical movement made by the first experiencing entity after experiencing the first procedure, training, at the biomechanical model custodian system, a learning engine using the received first experiencing entity data, accessing, at the biomechanical model custodian system, second experiencing entity data including third biomechanical movement data indicative of the first type of biomechanical movement made by a second experiencing entity prior to experiencing a second procedure on at least one anatomical feature of the second experiencing entity, after the training, predicting, using the learning engine at the biomechanical model custodian system and the accessed second experiencing entity data, achievement data for the second experiencing entity including fourth biomechanical movement data indicative of the first type of biomechanical movement predicted to be made by the second experiencing entity after experiencing the second procedure, detecting, with the biomechanical model custodian system, that the predicted achievement data for the second experiencing entity satisfies a rule, in response to the detecting, generating, with the biomechanical model custodian system, control data associated with the satisfied rule, and controlling a functionality of a managed element of the biomechanical model custodian system using the generated control data.

As another example, a method for managing biomechanical achievements using a biomechanical custodian system is provided that may include receiving, at the biomechanical custodian system, first experiencing entity data including first biomechanical movement data indicative of a first type of biomechanical movement made by a first experiencing entity prior to experiencing a first procedure on at least one anatomical feature of the first experiencing entity and second biomechanical movement data indicative of the first type of biomechanical movement made by the first experiencing entity after experiencing the first procedure, accessing, at the biomechanical custodian system, second experiencing entity data including third biomechanical movement data indicative of the first type of biomechanical movement made by a second experiencing entity prior to experiencing a second procedure on at least one anatomical feature of the second experiencing entity and fourth biomechanical movement data indicative of the first type of biomechanical movement made by the second experiencing entity after experiencing the second procedure, determining, with the biomechanical custodian system, that the accessed third biomechanical movement data is similar to the received first biomechanical movement data, in response to the determining, comparing, with the biomechanical custodian system, the accessed fourth biomechanical movement data to the received second biomechanical movement data, detecting, with the biomechanical custodian system, that the comparing satisfies a rule, in response to the detecting, generating, with the biomechanical custodian system, control data associated with the satisfied rule, and controlling a functionality of a managed element of the biomechanical custodian system using the generated control data.

As yet another example, a method for managing biomechanical achievements using a biomechanical model custodian system is provided that may include receiving, at the biomechanical model custodian system, condition category data for at least one condition category for a first condition of a first experiencing entity and achievement data for an actual achievement of the first experiencing entity for the first condition, training, at the biomechanical model custodian system, a learning engine using the received condition category data and the received achievement data, accessing, at the biomechanical model custodian system, condition category data for the at least one condition category for a second condition of a second experiencing entity, after the training, predicting an achievement of the second experiencing entity for the second condition, using the learning engine at the biomechanical model custodian system, with the accessed condition category data for the second condition, detecting, with the biomechanical model custodian system, that the predicted achievement satisfies a rule, in response to the detecting, generating, with the biomechanical model custodian system, control data associated with the satisfied rule, and controlling a functionality of a managed element of the biomechanical model custodian system using the generated control data.

As yet another example, a method for managing biomechanical achievements using a biomechanical custodian system is provided that may include receiving, at the biomechanical custodian system, condition category data for at least one condition category for a first condition of a first experiencing entity and achievement data for an actual achievement of the first experiencing entity for the first condition, accessing, at the biomechanical custodian system, condition category data for the at least one condition category for a second condition of a second experiencing entity and achievement data for an actual achievement of the second experiencing entity for the second condition, determining, with the biomechanical custodian system, that the accessed condition category data meets a similarity threshold with respect to the received condition category data, in response to the determining, comparing, with the biomechanical custodian system, the accessed achievement data to the received achievement data, detecting, with the biomechanical custodian system, that the comparing satisfies a rule, in response to the detecting, generating, with the biomechanical custodian system, control data associated with the satisfied rule, and controlling a functionality of a managed element of the biomechanical custodian system using the generated control data.

As yet another example, a method for managing biomechanical achievements using a biomechanical model custodian system including a global positioning subsystem is provided that may include receiving, at the biomechanical model custodian system, first experiencing entity data including first biomechanical movement data indicative of a first type of biomechanical movement made by a first experiencing entity while moving over a first period of time and first achievement data indicative of a first distance traveled by the first experiencing entity while moving over the first period of time, as determined by the global positioning subsystem, training, at the biomechanical model custodian system, a learning engine using the received first experiencing entity data, accessing, at the biomechanical model custodian system, second experiencing entity data including second biomechanical movement data indicative of the first type of biomechanical movement made by a second experiencing entity while moving over a second period of time, and, after the training, predicting, using the learning engine at the biomechanical model custodian system and the accessed second experiencing entity data, second achievement data indicative of a second distance traveled by the second experiencing entity while moving over the second period of time.

As yet another example, a method for managing biomechanical achievements using a biomechanical model custodian system is provided that may include receiving, at the biomechanical model custodian system, first experiencing entity data including first biomechanical movement data indicative of a first type of biomechanical movement made by the first experiencing entity while moving over a first period of time and first achievement data indicative of a first distance traveled by the first experiencing entity while moving over the first period of time, training, at the biomechanical model custodian system, a learning engine using the received first experiencing entity data, accessing, at the biomechanical model custodian system, second experiencing entity data including second biomechanical movement data indicative of the first type of biomechanical movement made by a second experiencing entity while moving over a second period of time, after the training, predicting, using the learning engine at the biomechanical model custodian system and the accessed second experiencing entity data, second achievement data indicative of a second distance traveled by the second experiencing entity while moving over the second period of time, detecting, with the biomechanical model custodian system, that the predicted second achievement data for the second experiencing entity satisfies a rule, in response to the detecting, generating, with the biomechanical model custodian system, control data associated with the satisfied rule, and controlling a functionality of a managed element of the biomechanical model custodian system using the generated control data.

As yet another example, a method for managing biomechanical achievements using a biomechanical model custodian system is provided that may include receiving, at the biomechanical model custodian system, first experiencing entity data including first biomechanical movement data indicative of a first type of biomechanical movement made by the first experiencing entity while moving over a first period of time and first achievement data indicative of a first distance traveled by the first experiencing entity while moving over the first period of time, training, at the biomechanical model custodian system, a learning engine using the received first experiencing entity data, accessing, at the biomechanical model custodian system, second experiencing entity data including second biomechanical movement data indicative of the first type of biomechanical movement made by a second experiencing entity while moving over a second period of time, and, after the training, predicting, using the learning engine at the biomechanical model custodian system and the accessed second experiencing entity data, second achievement data indicative of a second distance traveled by the second experiencing entity while moving over the second period of time, wherein the first biomechanical movement data is indicative of the first type of biomechanical movement made by the first experiencing entity while moving over the first period of time and a second type of biomechanical movement made by the first experiencing entity while moving over the first period of time, the second biomechanical movement data is indicative of the first type of biomechanical movement made by the second experiencing entity while moving over the second period of time and the second type of biomechanical movement made by the second experiencing entity while moving over the second period of time, and the first type of biomechanical movement is different than the second type of biomechanical movement.

This Summary is provided to summarize some example embodiments, so as to provide a basic understanding of some aspects of the subject matter described in this document. Accordingly, it will be appreciated that the features described in this Summary are only examples and should not be construed to narrow the scope or spirit of the subject matter described herein in any way. Unless otherwise stated, features described in the context of one example may be combined or used with features described in the context of one or more other examples. Other features, aspects, and advantages of the subject matter described herein will become apparent from the following Detailed Description, Figures, and Claims.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals may identify like elements, and in which:

FIGS. 1A-1C show front, back, and side views of a base layer of an exosuit according to an embodiment;

FIGS. 1D-1F show front, back, and side views, respectively, of a power layer according to an embodiment;

FIGS. 1G and 1H show respective front and back views of a human male's musculature anatomy, according to an embodiment;

FIGS. 1I and 1J show front and side views of an illustrative exosuit having several power layer segments that approximate many of the muscles shown in FIGS. 1G and 1H, according to various embodiments;

FIGS. 2A and 2B show front and back view of illustrative exosuit according to an embodiment;

FIG. 3 shows an illustrative symbiosis exosuit system according to an embodiment;

FIG. 4 shows illustrative process for implementing a symbiosis exosuit system according to an embodiment;

FIG. 5 shows an illustrative diagram of different control modules that may be implemented by an exosuit according to an embodiment;

FIG. 6 shows an illustrative block diagram according to an embodiment;

FIG. 7 shows a system including an example suit according to an embodiment;

FIG. 8 illustrates an example exosuit according to an embodiment;

FIG. 9 is a schematic illustrating elements of an exosuit and a hierarchy of control or operating the exosuit according to an embodiment;

FIG. 10 is a schematic representation of a system using activity monitor sensing of an exosuit according to an embodiment;

FIG. 11 is a schematic view of an illustrative system for managing biomechanical achievements according to an embodiment;

FIG. 12 is a schematic view of an illustrative portion of the system of FIG. 11 according to an embodiment;

FIG. 13 is a flowchart of an illustrative process for managing biomechanical achievements according to an embodiment;

FIGS. 14-19 show plots of various biomechanical gait markers over time according to some embodiments;

FIG. 20 shows a step-length distribution of various sensed run segments according to an embodiment;

FIG. 21 shows a plot of a correlation between average step size and pelvic transverse rotation according to an embodiment;

FIG. 22 shows a plot of various runners' model output error versus number of runs used for model training according to an embodiment;

FIG. 23 shows a plot of accumulated distance over the course of a run versus predicted estimated distance data for various data source types according to an embodiment;

FIG. 24 shows a comparison of test errors between a linear regression model trained on a population, a multiple-perceptron model trained on a population, and a linear regression model trained on an individual user according to an embodiment; and

FIGS. 25-33 are flowcharts of illustrative processes for managing biomechanical achievements according to some embodiments.

### DETAILED DESCRIPTION

In the following description, numerous specific details are set forth regarding the systems, methods, and media of the disclosed subject matter and the environment in which such systems, methods, and media may operate, and/or the like, in order to provide a thorough understanding of the disclosed subject matter. It can be apparent to one skilled in the art, however, that the disclosed subject matter may be practiced without such specific details, and that certain features, which are well known in the art, are not described in detail in order to avoid complication of the disclosed subject matter. In addition, it can be understood that the examples provided below are exemplary, and that it is contemplated that there are other systems, methods, and media that are within the scope of the disclosed subject matter.

An exosuit or any other suitable sensor assembly worn by a user can be utilized by a system to monitor several movement factors that may characterize the user's movement and any changes in the user's movement with a high degree of specificity that may enable various system algorithms and/or models to predict or otherwise determine one or more biomechanical achievements of the user, such as recovery from a particular type of event (e.g., surgery or therapy procedure) and/or distance traveled (e.g., without using any global positioning system capabilities). In addition, an exosuit can provide useful feedback in response to such determinations.

In the descriptions that follow, an exosuit or assistive exosuit may be a suit that can be worn by a wearer on the outside of his or her body. It may be worn under the wearer's normal clothing, over their clothing, between layers of clothing, or may be the wearer's primary clothing itself. The exosuit may be supportive and/or assistive, as it can physically support and/or assist the wearer in performing particular activities, or can provide other functionality, such as communication to the wearer through physical expressions to the body, engagement of the environment, capturing of information from the wearer, and/or the like. In some embodiments, a powered exosuit system can include several subsystems or layers. In some embodiments, the powered exosuit system can include more or fewer subsystems or layers. The subsystems or layers can include a base layer, a stability layer, a power layer, a sensor and controls layer, a covering layer, and/or a user interface/user experience (UI/UX) layer.

The base layer may provide one or more interfaces between the exosuit system and the wearer's body. The base layer may be adapted to be worn directly against the wearer's skin, between undergarments and outer layers of clothing, over outer layers of clothing or a combination thereof, or the base layer may be designed to be worn as primary clothing itself In some embodiments, the base layer can be adapted to be both comfortable and unobtrusive, as well as to comfortably and efficiently transmit loads from the stability layer and power layer to the wearer's body in order to provide the desired assistance. The base layer can typically include several different material types to achieve these purposes. Elastic materials may provide compliance to conform to the wearer's body and may allow for ranges of movement. The innermost layer may typically be adapted to grip the wearer's skin, undergarments, or clothing so that the base layer does not slip as loads are applied. Substantially inextensible materials may be used to transfer loads from the stability layer and power layer to the wearer's body. These materials may be substantially inextensible in one axis, yet flexible or extensible in other axes such that the load transmission may be along preferred paths. The load transmission paths may be optimized to distribute the loads across regions of the wearer's body to minimize the forces felt by the wearer, while providing efficient load transfer with minimal loss and not causing the base layer to slip. Collectively, this load transmission configuration within the base layer may be referred to as a load distribution member. Load distribution members may refer to flexible elements that distribute loads across a region of the wearer's body. Examples of load distribution members can be found in International Application Publication No. WO 2016/138264, titled “Flexgrip,” the contents of which are incorporated herein by reference.

Load distribution members may incorporate one or more catenary curves to distribute loads across the wearer's body. Multiple load distribution members or catenary curves may be joined with pivot points, such that as loads are applied to the structure, the arrangement of the load distribution members may pivot, tighten, and/or constrict on the body to increase the gripping strength. Compressive elements, such as battens, rods, or stays, may be used to transfer loads to different areas of the base layer for comfort or structural purposes. For example, a power layer component may terminate in the middle back due to its size and orientation requirements, however the load distribution members that may anchor the power layer component may reside on the lower back. In this case, one or more compressive elements may transfer the load from the power layer component at the middle back to the load distribution member at the lower back.

Load distribution members may be constructed using multiple fabrication and textile application techniques. For example, a load distribution member can be constructed from a layered woven 45°/90° with bonded edge, spandex tooth, organza (poly) woven 45°/90° with bonded edge, organza (cotton/silk) woven 45°/90°, and/or Tyvek (non-woven). A load distribution member may be constructed using knit and lacing or horse hair and spandex tooth. A load distribution member may be constructed using channels and/or laces.

A base layer may include a flexible underlayer that may be constructed to compress against a portion of the wearer's body, either directly to the skin or to a clothing layer, and also may provide a relatively high grip surface for one or more load distribution members to attach thereto. Load distribution members can be coupled to an underlayer to facilitate transmission of shears or other forces from the members, via the flexible underlayer, to skin of a body segment or to clothing worn over the body segment, to maintain the trajectories of the members relative to such a body segment, and/or to provide some other functionality. Such a flexible underlayer could have a flexibility and/or compliance that differs from that of the member (e.g., that is less than that of the members, at least in a direction along the members), such that the member can transmit forces along its length and evenly distribute shear forces and/or pressures, via the flexible underlayer, to skin of a body segment to which a flexible body harness may be mounted.

Further, such a flexible underlayer can be configured to provide additional functionality. The material of the flexible underlayer could include anti-bacterial, anti-fungal, or other agents (e.g., silver nanoparticles) to prevent the growth of microorganisms. The flexible underlayer can be configured to manage the transport of heat and/or moisture (e.g., sweat) from a wearer to improve the comfort and efficiency of activity of the wearer. The flexible underlayer can include straps, seams, hook-and-loop fasteners, clasps, zippers, or other elements that may be configured to maintain a specified relationship between elements of the load distribution members and aspects of a wearer's anatomy. The underlayer can additionally increase the ease with which a wearer can don and/or doff the flexible body harness and/or a system (e.g., a flexible exosuit system) or garment that includes the flexible body harness. The underlayer can additionally be configured to protect the wearer from ballistic weapons, sharp edges, shrapnel, or other environmental hazards (e.g., by including panels or flexible elements of para-aramid or other high-strength materials).

The base layer can additionally include features, such as size adjustments, openings, and electro-mechanical integration features to improve ease of use and comfort for the wearer.

Size adjustment features may permit the exosuit to be adjusted to the wearer's body. The size adjustments may allow the suit to be tightened or loosened about the length or circumference of the torso or limbs. The adjustments may include lacing (e.g., the Boa system), webbing, elastic, hook-and-loop, and/or other fasteners. Size adjustment may be accomplished by the load distribution members themselves, for example, as they may constrict onto the wearer when loaded. In one example, the torso circumference may be tightened with corset-style lacing, the legs tightened with hook-and-loop in a double-back configuration, and the length and shoulder height adjusted with webbing and tension-lock fasteners, such as cam-locks, D-rings, or the like. The size adjustment features in the base layer may be actuated by the power layer to dynamically adjust the base layer to the wearer's body in different positions, in order to maintain consistent pressure and comfort for the wearer. For example, the base layer may be configured to tighten on the thighs when the wearer is standing and loosen when the wearer is sitting such that the base layer may not excessively constrict the thighs when the wearer is seated. The dynamic size adjustment may be controlled by the sensor and controls layer, for example, by detecting pressures or forces in the base layer and actuating the power layer to consistently attain the desired force or pressure. This feature may not necessarily cause the suit to provide physical assistance, but can create a more comfortable experience for the wearer, or may allow the physical assistance elements of the suit to perform better or differently depending on the purpose of the movement assistance.

Opening features in the base layer may be provided to facilitate donning (e.g., putting the exosuit on) and doffing (e.g., taking the exosuit off) for the wearer. Opening features may include zippers, hook-and-loop, snaps, buttons, and/or other textile fasteners. In one example, a front, central zipper may provide an opening feature for the torso, while hook-and-loop fasteners may provide opening features for the legs and shoulders. In this case, the hook-and-loop fasteners may provide both opening and adjustment features. In other examples, the exosuit may simply have large openings, for example around the arms or neck, and elastic panels that may allow the suit to be donned and doffed without specific closure mechanisms. A truncated load distribution member may be simply extended to tighten on the wearer's body. Openings may be provided to facilitate toileting so the user can keep the exosuit on, but only have to remove or open a relatively small portion to use the bathroom.

Electro-mechanical integration features may attach components of the stability layer, power layer, and/or sensor and controls layer into the base layer for integration into the exosuit. The integration features may be for mechanical, structural, comfort, protective, and/or cosmetic purposes. Structural integration features may anchor components of the other layers to the base layer. For the stability and power layers, the structural integration features may provide for load-transmission to the base layer and load distribution members, and may accommodate specific degrees of freedom at the attachment point. For example, a snap or rivet anchoring a stability or power layer element may provide both load transmission to the base layer, as well as a pivoting degree of freedom. Stitched, adhesive, and/or bonded anchors may provide load transmission with or without the pivoting degree of freedom. A sliding anchor, for example, along a sleeve or rail, may provide a translational degree of freedom. Anchors may be separable, such as with snaps, buckles, clasps, and/or hooks, or may be inseparable, such as with stitching, adhesives, and/or other bonding. Size adjustment features, such as described above, may allow adjustment and customization of the stability and power layers, for example, to adjust the tension of spring or elastic elements in the passive layer, or to adjust the length of actuators in the power layer.

Other integration features, such as loops, pockets, and/or mounting hardware, may simply provide attachment to components that may not have significant load transmission requirements, such as batteries, circuit boards, sensors, and/or cables. In some cases, components may be directly integrated into textile components of the base layer. For example, cables or connectors may include conductive elements that may be directly woven, bonded, and/or otherwise integrated into the base layer.

Electromechanical integration features may also protect or cosmetically hide components of the stability, power, and/or sensor and controls layers. Elements of the stability layer (e.g., elastic bands or springs), power layer (e.g., flexible linear actuators or twisted string actuators), and/or sensor and controls layer (e.g., cables) may travel through sleeves, tubes, and/or channels that may be integrated into the base layer, which can both conceal and protect these components. The sleeves, tubes, and/or channels may also permit motion of the component, for example during actuation of a power layer element. The sleeves, channels, and/or tubes may include resistance to collapse, ensuring that the component remains free and uninhibited within.

Enclosures, padding, fabric coverings, and/or the like may be used to further integrate components of other layers into the base layer for cosmetic, comfort, and/or protective purposes. For example, components, such as motors, batteries, cables, and/or circuit boards, may be housed within an enclosure, fully or partially covered, and/or surrounded in padded material, such that the components may not cause discomfort to the wearer, may be visually unobtrusive, may be integrated into the exosuit, and/or may be protected from the environment. Opening and closing features may additionally provide access to these components for service, removal, and/or replacement.

In some cases, particularly for exosuits that may be configurable for either provisional use or testing, a tether may allow for some electronic and mechanical components to be housed off the suit. In one example, electronics, such as circuit boards and batteries, may be over-sized, which may allow for added configurability or data capture. If the large size of these components makes it undesirable to mount them on the exosuit, they may be located separately from the suit and connected via a physical or wireless tether. Larger, over-powered motors may be attached to the suit via flexible drive linkages that may allow actuation of the power layer without requiring large motors to be attached to the suit. Such over-powered configurations may allow optimization of exosuit parameters without constraints requiring all components to be attached or integrated into the exosuit.

Electro-mechanical integration features may also include wireless communication. For example, one or more power layer components may be placed at different locations on the exosuit. Rather than utilizing physical electrical connections to the sensors and controls layer, the sensor and controls layer may communicate with the one or more power layer components via any suitable wireless communication protocols, such as Bluetooth, ZigBee, ultrawide band, or any other suitable communication protocol. This may reduce the electrical interconnections within the suit. Each of the one or more power layer components may additionally incorporate a local battery, such that each power layer component or group of power layer components may be independently powered units that may not require direct electrical interconnections to other areas of the exosuit.

The stability layer may provide passive mechanical stability and assistance to the wearer. The stability layer may include one or more passive (e.g., non-powered) spring or elastic elements that may generate forces and/or store energy to provide stability or assistance to the wearer. An elastic element can have an un-deformed, least-energy state. Deformation (e.g., elongation) of the elastic element may store energy and/or generate a force that may be oriented to return the elastic element toward its least-energy state. For example, elastic elements approximating hip flexors and/or hip extensors may provide stability to the wearer in a standing position. As the wearer deviates from the standing position, the elastic elements may be deformed, generating forces that may stabilize the wearer and assist maintaining the standing position. In another example, as a wearer moves from a standing to seated posture, energy may be stored in one or more elastic elements, generating a restorative force that may assist the wearer when moving from the seated to standing position. Similar passive, elastic elements may be adapted to the torso or other areas of the limbs to provide positional stability or assistance for moving to a position where the elastic elements may be in their least-energy state.

Elastic elements of the stability layer may be integrated to parts of the base layer or be an integral part of the base layer. For example, elastic fabrics containing spandex or similar materials may serve as a combination base/stability layer. Elastic elements may also include discrete components, such as springs or segments of elastic material, such as silicone or elastic webbing, that may be anchored to the base layer for load transmission at discrete points, such as described above.

A stability layer may be adjusted, such as described above, both to adapt to the wearer's size and individual anatomy, as well as to achieve a desired amount of pre-tension or slack in components of the stability layer in specific positions. For example, some wearers may prefer more pre-tension to provide additional stability in the standing posture, while others may prefer more slack, so that the passive layer may not interfere with other activities, such as ambulation.

A stability layer may interface with the power layer to engage, disengage, and/or adjust the tension or slack in one or more elastic elements. In one example, when the wearer is in a standing position, the power layer may pre-tension one or more elastic elements of the stability layer to a desired amount for maintaining stability in that position. The pre-tension may be further adjusted by the power layer for different positions or activities. In some embodiments, the elastic elements of the stability layer may be able to generate at least 5 pounds force, and preferably at least 50 pounds force when elongated.

A power layer can provide active, powered assistance to the wearer, as well as electromechanical clutching to maintain components of the power or stability layers in a desired position or tension. The power layer can include one or more flexible linear actuators (FLAs). An FLA may be a powered actuator that may be capable of generating a tensile force between two attachment points, over a given stroke length. An FLA may be flexible, such that it can follow a contour, for example, around a body surface, and therefore the forces at the attachment points may not necessarily be aligned. In some embodiments, one or more FLAs can include one or more twisted string actuators. In the descriptions that follow, FLA may refer to a flexible linear actuator that may exert a tensile force, contracts, and/or shortens when actuated. The FLA may be used in conjunction with a mechanical clutch that may lock the tension force generated by the FLA in place so that the FLA motor may not have to consume power to maintain the desired tension force. Examples of such mechanical clutches are discussed below. In some embodiments, FLAs can include one or more twisted string actuators or flexdrives, as described in further detail in U.S. Pat. No. 9,266,233, titled “Exosuit System,” the contents of which are incorporated herein by reference. FLAs may also be used in connection with electrolaminate clutches, which are also described in the U.S. Pat. No. 9,266,233. The electrolaminate clutch (e.g., clutches that may be configured to use electrostatic attraction to generate controllable forces between clutching elements) may provide power savings by locking a tension force without requiring the FLA to maintain the same force.

The powered actuators, or FLAs, may be arranged on the base layer, connecting different points on the body, to generate forces for assistance with various activities. The arrangement can often approximate the wearer's muscles, in order to naturally mimic and assist the wearer's own capabilities. For example, one or more FLAs may connect the back of the torso to the back of the legs, thus approximating the wearer's hip extensor muscles. Actuators approximating the hip extensors may assist with activities, such as standing from a seated position, sitting from a standing position, walking, and/or lifting. Similarly, one or more actuators may be arranged approximating other muscle groups, such as the hip flexors, spinal extensors, abdominal muscles, and/or muscles of the arms and/or legs.

One or more FLAs approximating a group of muscles may be capable of generating at least 10 pounds over at least a ½ inch stroke length within 4 seconds. In some embodiments, one or more FLAs approximating a group of muscles may be capable of generating at least 250 pounds over a 6 inch stroke within ½ second. Multiple FLAs, arranged in series or parallel, may be used to approximate a single group of muscles, with the size, length, power, and/or strength of the FLAs optimized for the group of muscles and activities for which they are utilized.

The sensor and controls layer may capture data from the suit and wearer, utilize the sensor data and other commands to control the power layer based on the activity being performed, and/or provide suit and wearer data to the UX/UI layer for control and informational purposes.

Sensors, such as encoders or potentiometers, may measure the length and rotation of the FLAs, while force sensors may measure the forces applied by the FLAs, and while inertial measurement units (IMUs) may measure and enable computation of kinematic data (e.g., positions, velocities, and/or accelerations) of points on the suit and wearer. These data may enable inverse dynamics calculations of kinetic information (e.g., forces, torques) of the suit and wearer. Electromyographic (EMG) sensors may detect the wearer's muscle activity in specific muscle groups. Electronic control systems (ECSs) on the suit may use parameters measured by the sensor layer to control the power layer. Data from the IMUs may indicate both the activity being performed, as well as the speed and intensity. For example, a pattern of IMU and/or EMG data may enable the ECS to detect that the wearer is walking at a specific pace. This information may then enable the ECS, utilizing the sensor data, to control the power layer in order to provide the appropriate assistance to the wearer. Stretchable sensors may be used, for example, as a strain gauge, to measure the strain of the elements in the stability layer, and thereby may predict the forces in the elastic elements of the stability layer. Stretchable sensors may be embedded in the base layer or grip layer and may be used to measure the motion of the fabrics in the base layer and the motion of the body.

Data from the sensor layer may be further provided to the UX/UI layer, such as for feedback and information to the wearer, caregivers, and/or service providers.

A UX/UI layer may include the wearer's and/or others' interaction and experience with the exosuit system. This layer may include controls of the suit itself, such as initiation of activities, as well as feedback to the wearer and caregivers. A retail or service experience may include steps of fitting, calibration, training, and/or maintenance of the exosuit system. Other UX/UI features may include additional lifestyle features, such as electronic security, identity protection, and/or health status monitoring.

An assistive exosuit can have a user interface that may be used for the wearer to instruct the suit which activity is to be performed, as well as the timing of the activity. In one example, a user may manually instruct the exosuit to enter an activity mode via one or more user interface features, such as one or more buttons, a keypad, or a tethered device, such as a mobile phone. In another example, the exosuit may detect initiation of an activity from the sensor and controls layer, as described previously. In yet another example, the user may speak a desired activity mode to the suit, which can interpret the spoken request to set the desired mode. The suit may be pre-programmed to perform the activity for a specific duration, until another command is received from the wearer, or until the suit detects that the wearer has ceased the activity. The suit may include cease activity features that, when activated, may cause the suit to cease all activity. The cease activity features can take into account the motion being performed, and can disengage in a way that may take into account the user's position and motion, and may safely return the user to an unloaded state in a safe posture.

The exosuit may have a UX/UI controller that may be defined as a node on another user device, such as a computer or mobile smart phone. The exosuit may also be the base for other accessories. For example, the exosuit may include a cell phone chip so that the suit may be capable of receiving both data and voice commands directly similar to a cell phone, and can communicate information and voice signals through such a node. The exosuit control architecture can be configured to allow for other devices to be added as accessories to the exosuit. For example, a video screen may be connected to the exosuit to show images that are related to the use of the suit. The exosuit may be used to interact with smart household devices, such as door locks, or can be used to turn on smart televisions and adjust channels and other settings. In these modes, the physical assist of the suit can be used to augment or create physical or haptic experiences for the wearer that may be related to communication with these devices. For instance, an email could have a pat on the back as a form of physical emoji that when inserted in the email causes the suit to physically tap the wearer or perform some other type of physical expression to the user that may add emphasis to the written email.

The exosuit may provide visual, audio, and/or haptic feedback and/or cues to inform the user of various exosuit operations. For example, the exosuit may include vibration motors to provide haptic feedback. As a specific example, two haptic motors may be positioned near the front hip bones to inform the user of suit activity when performing a sit-to-stand assistive movement. In addition, two haptic motors may be positioned near the back hip bones to inform the user of suit activity when performing a stand-to-sit assistive movement. The exosuit may include one or more light emitting diodes (LEDs) to provide visual feedback or cues. For example, LEDs may be placed near the left and/or right shoulders within the peripheral vision of the user. The exosuit may include a speaker or buzzer to provide audio feedback or cues.

In other instances, the interaction of the FLAs with the body through the body harness and otherwise can be used as a form of haptic feedback to the wearer, where changes in the timing of the contraction of the FLAs can indicate certain information to the wearer. For instance, the number and/or strength of tugs of the FLA on the waist could indicate the amount of battery life remaining or that the suit has entered a ready state for an impending motion.

The control of the exosuit may also be linked to the sensors that may be measuring the movement of the wearer, or other sensors, for instance on the suit of another person, or sensors in the environment. The motor commands described herein may all be activated or modified by this sensor information. In this example, the suit can exhibit its own reflexes such that the wearer, through intentional and/or unintentional motions, may cue the motion profile of the suit. When sitting, for further example, the physical movement of leaning forward in the chair, as if to indicate an intention to stand up, can be sensed by the suit IMUs and may be used to trigger a sit to stand motion profile. In one embodiment, the exosuit may include sensors (e.g., one or more electroencephalograph (EEG) sensors) that may be able to monitor brain activity that may be used to detect a user's desire to perform a particular movement. For example, if the user is sitting down, an EEG sensor may sense the user's desire to stand up and cause the exosuit to prime itself to assist the user in a sit-to-stand assistive movement.

The suit may make sounds or provide other feedback, for instance through quick movements of the motors, as information to the user that the suit has received a command or to describe to the user that a particular motion profile can be applied. In the above reflex control example, the suit may provide a high pitch sound and/or a vibration to the wearer to indicate that it is about to start the movement. This information can help the user to be ready for the suit movements, improving performance and/or safety. Many types of cues may be possible for all movements of the suit.

Control of the suit may include the use of machine learning techniques to measure movement performance across many instances of one or many wearers of suits connected via the internet, where the calculation of the best control motion for optimizing performance and improving safety for any one user may be based on the aggregate information in all or a subset of the wearers of the suit. The machine learning techniques can be used to provide user specific customization for exosuit assistive movements. For example, a particular user may have an abnormal gait (e.g., due to a car accident) and thus may be unable to take even strides. The machine learning may detect this abnormal gait and compensate accordingly for it.

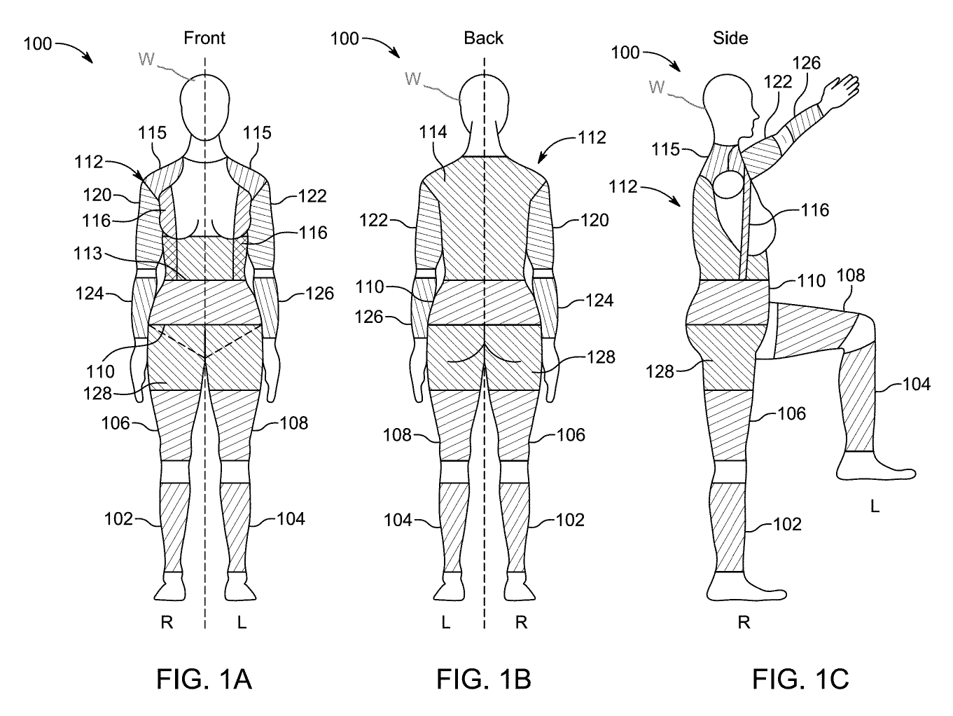

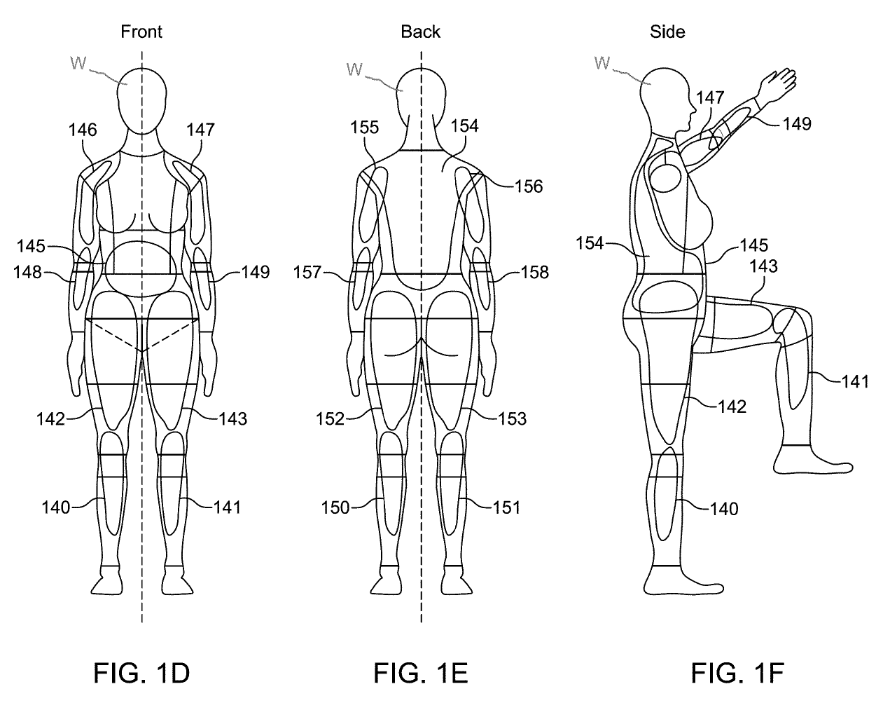

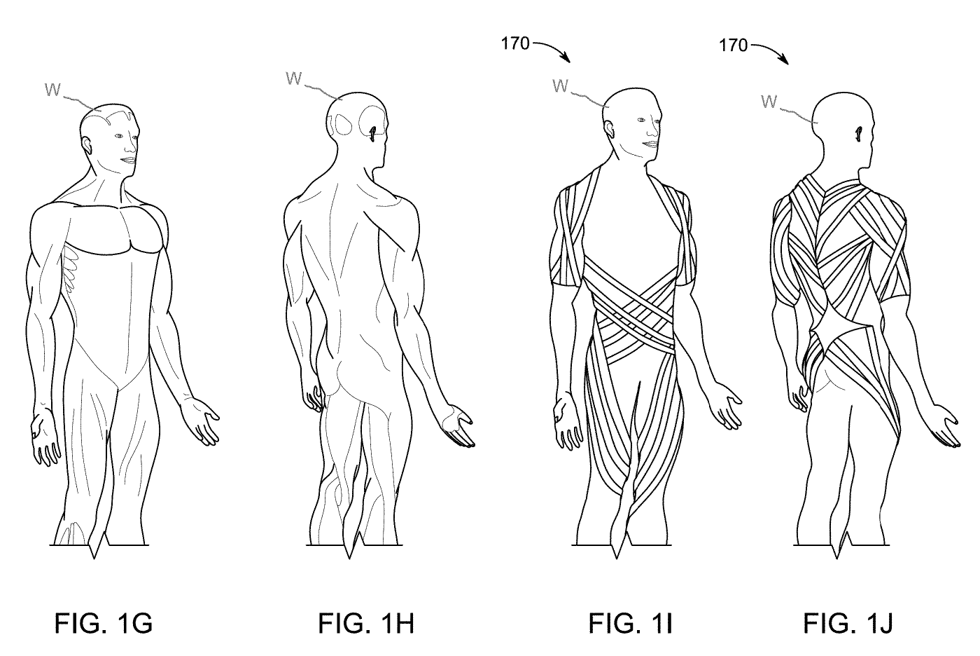

FIGS. 1A-1C show front, back, and side views of a base layer 100 of an exosuit according to an embodiment. Base layer 100 may be worn by a wearer W (e.g., a human body as shown) as a single piece or as multiple pieces. As shown, base layer 100 is shown to represent multiple pieces that can serve as load distribution members (LDMs) for a power layer (e.g., as shown in FIGS. 1D-1F). Base layer 100 and any LDMs thereof can cover or occupy any part of a human body or of any other suitable type of wearer as desired. The LDMs shown in FIGS. 1A-1C are merely illustrative of a few potential locations and it should be appreciated that additional LDMs may be added or certain LDMs may be omitted.

Base layer 100 can include calf LDMs 102 and 104 that may be secured around the calf region or lower leg portion of the wearer. Calf LDMs 102 and 104 are shown to be positioned between the knees and the ankles, but this is merely illustrative. If desired, calf LDMs 102 and 104 can also cover the foot and ankle and/or the knee of the wearer.

Base layer 100 can include thigh LDMs 106 and 108 that may be secured around the thigh region of the wearer. Thigh LDMs 106 and 108 are shown to be positioned between the knees and an upper region of the thighs. In some embodiments, thigh LDMs 106 and 108 and calf LDMs 102 and 104, respectively, may be merged together to form leg LDMs that may cover the entirety of the legs and/or feet of the wearer.

Base layer 100 can include a hip LDM 110 that may be secured around a hip region of the wearer. LDM 110 may be bounded such that it may remain positioned above the toileting regions of the human. Such bounding may make toileting relatively easy for the human as it may not be required to remove base layer 100 to use the bathroom. In some embodiments, LDM 110 may be attached to thigh LDMs 106 and 108, but the toileting regions may remain uncovered. In another embodiment, a removable base layer portion may exist between LDM 110 and thigh LDMs 106 and 108.

Base layer 100 can include an upper torso LDM 112 that may be secured around an upper torso region of the wearer. Upper torso LDM 112 may include a waist LDM 113, a back LDM 114, a shoulder LDM 115, and shoulder strap LDMs 116. Waist LDM 113, back LDM 114, shoulder LDM 115, and shoulder strap LDMs 116 may be integrally formed to yield upper torso LDM 112. In some embodiments, a chest LDM (not shown) may also be integrated into upper torso LDM 112. Female specific exosuits may have built in bust support for the chest LDM.

Base layer 100 can include upper arm LDMs 120 and 122 and lower arm LDMs 124 and 126. Upper arm LDMs 120 and 122 may be secured around bicep/triceps region of the aim and can occupy space between the shoulder and the elbow. Lower arm LDMs 124 and 126 may be secured around the forearm region of the arm and can occupy the space between the elbow and the wrist. If desired, upper arm LDM 120 and lower arm LDM 124 may be integrated to form an arm LDM, and upper arm LDM 122 and lower arm LDM 126 may be integrated to form another arm LDM. In some embodiments, arm LDMs 120, 122, 124, and 126 may form part of upper torso LDM 112.

Base layer 100 can include a gluteal/pelvic LDM 128 that may be secured the gluteal and pelvic region of the wearer. LDM 128 may be positioned between thigh LDMs 106 and 108 and hip LDM 110. LDM 128 may have removable portions, such as buttoned or zippered flaps, that may permit toileting. Although not shown in FIGS. 1A-1C, LDMs may exist for the feet, toes, neck, head, hands, fingers, elbows, and/or any other suitable body part.

As explained above, the LDMs may serve as attachment points for components of a power layer. In particular, the components that may provide muscle assistance movements may typically need to be secured in at least two locations on the body. This way, when the flexible linear actuators are engaged, the contraction of the actuator can apply a force between the at least two locations on the body. With LDMs strategically placed around the body, the power layer can also be strategically placed thereon to provide any number of muscle assistance movements. For example, the power layer may be distributed across different LDMs or within different regions of the same LDM to approximate any number of different muscles or muscle groups. The power layer may approximate muscle groups such as the abdominals, adductors, dorsal muscles, shoulders, arm extensors, wrist extensors, gluteals, arm flexors, wrist flexors, scapulae fixers, thigh flexors, lumbar muscles, surae, pectorals, quadriceps, and/or trapezii.

FIGS. 1D-1F show front, back, and side views, respectively, of a power layer according to an embodiment. The power layer is shown as multiple segments distributed across and within the various LDMs. As shown, the power layer can include power layer segments 140-158. One, some, or each power layer segment can include any number of flexible linear actuators. Some of the power layer segments may exist solely on the anterior side of the body, exist solely on the posterior side, start on the anterior side and wrap around to the posterior side, start on the posterior side and wrap around to the anterior side, or wrap completely around a portion of the body. Power layer segment (PLS) 140 may be secured to LDM 102 and LDM 106, and PLS 141 may be secured to LDM 104 and LDM 108. PLS 142 may be secured to LDM 106 and LDM 110 and/or LDM 114, and PLS 143 may be secured to LDM 108 and LDM 110 and/or LDM 114. PLS 145 may be secured to LDM 110 and LDM 113 and/or to LDM 114 or LDM 128. PLS 146 may be secured to LDM 115 and LDM 120, and PLS 147 may be secured to LDM 115 and LDM 122. PLS 148 may be secured to LDM 120 and LDM 124, and PLS 149 may be secured to LDM 122 and LDM 126.

PLS 150 may be secured to LDM 104 and LDM 108, and PLS 151 may be secured to LDM 102 and LDM 106. PLS 152 may be secured to LDM 106 and LDM 110 and/or to LDM 113, and PLS 153 may be secured to LDM 108 and LDM 110 and/or LDM 113. PLS 154 may be secured to LDM 112 and LDM 110. PLS 155 may be secured to LDM 112 and LDM 120, and PLS 156 may be secured to LDM 112 and LDM 122. PLS 157 may be secured to LDM 120 and LDM 124, and PLS 158 may be secured to LDM 122 and LDM 126.

It should be appreciated that the power layer segments are merely illustrative and that additional power layer segments may be added or that some segments may be omitted. In addition, the attachment points for the power layer segments are merely illustrative and that other attachment points may be used.

The human body has many muscles, including large and small muscles that are arranged in all sorts of different configuration. For example, FIGS. 1G and 1H show respective front and back views of a human male wearer W's musculature anatomy, which shows many muscles. In particular, the abdominals, adductors, dorsal muscles, shoulders, arm extensors, wrist extensors, gluteals, arm flexors, wrist flexors, scapulae fixers, thigh flexors, lumbar muscles, pectorals, quadriceps, and trapezii are all shown.

The LDMs may be designed so that they can accommodate different sizes of individuals who don the exosuit. For example, the LDMs may be adjusted to achieve the best fit. In addition the LDMs may be designed such that the location of the end points and the lines of action may be co-located with the bone structure of the user in such a way that the flexdrive placement on the exosuit system may be aligned with the actual muscle structure of the wearer for comfort, and the moment arms and forces generated by the flexdrive/exosuit system may feel aligned with the forces generated by the wearer's own muscles.

FIGS. 1I and 1J show front and side views of an illustrative exosuit 170 that may include several power layer segments that may approximate many of the muscles shown in FIGS. 1G and 1H of wearer W. The power layer segments are represented by the individual lines that span different parts of the body. These lines may represent specific flexible linear actuators (FLAs) or groups thereof that may work together to form the power layer segments that may be secured to the LDMs (not shown). As shown, the FLAs may be arrayed to replicate at least a portion of each of the abdominal muscles, dorsal muscles, shoulder muscles, arm extensor and flexor muscles, gluteal muscles, quadriceps muscles, thigh flexor muscles, and trapezii muscles. Thus, exosuit 170 exemplifies one of many possible different power layer segment arrangements that may be used in exosuits in accordance with embodiments discussed herein. These power layer segments may be arranged so that the moment arms and forces generated feel like forces being generated by the user's own muscles, tendons, and skeletal structure. Other possible power layer segment arrangements are illustrated and discussed below.

The power layer segments may be arranged such that they may include opposing pairs or groups, similar to the way human muscles are arranged in opposing pairs or groups of muscles. That is, for a particular movement, the opposing pairs or groups can include protagonist and antagonist muscles. While performing the movement, protagonist muscles may perform the work, whereas the antagonist muscles may provide stabilization and resistance to the movement. As a specific example, when a user is performing a curl, the biceps muscles may serve as the protagonist muscles and the triceps muscles may serve as the antagonist muscles. In this example, the power layer segments of an exosuit may emulate the biceps and triceps. When the biceps human muscle is pulling to bend the elbow, the exosuit triceps power layer segment can pull on the other side of the joint to resist bending of the elbow by attempting to extend it. The power layer segment can be, for example, either a FLA operating alone to apply the force and motion, or a FLA in series with an elastic element. In the latter case, the human biceps may be working against the elastic element, with the FLA adjusting the length and thereby the resistive force of the elastic element.

Thus, by arranging the power layer segments in protagonist and antagonist pairs, the power layers segments can mimic or emulate any protagonist and antagonist pairs of the human anatomy musculature system. This can be used to enable exosuits to provide assistive movements, alignment movements, and resistive movements. For example, for any exercise movement that requires activation of protagonist muscles, a subset of the power layer segments can emulate activation of antagonist muscles associated with that exercise movement to provide resistance.

The design flexibility of the LDMs and PLSs can enable exosuits to be constructed in accordance with embodiments discussed herein. Using exosuits, the power layer segments can be used to resist motion, assist motion, or align the user's form.

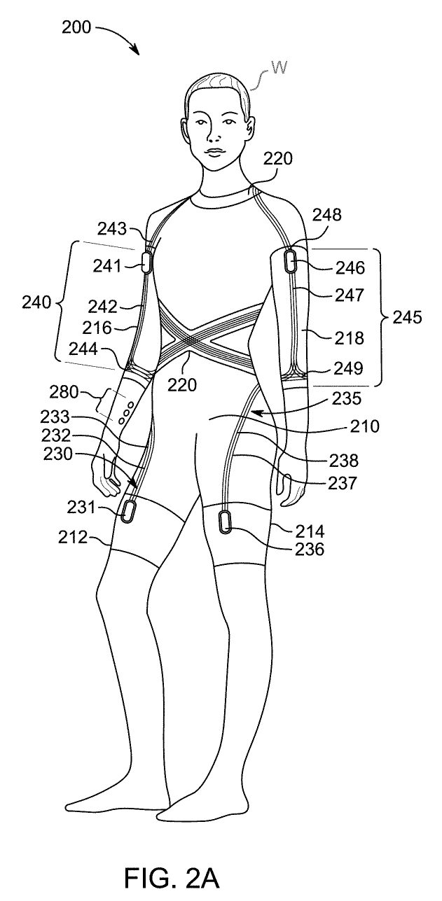

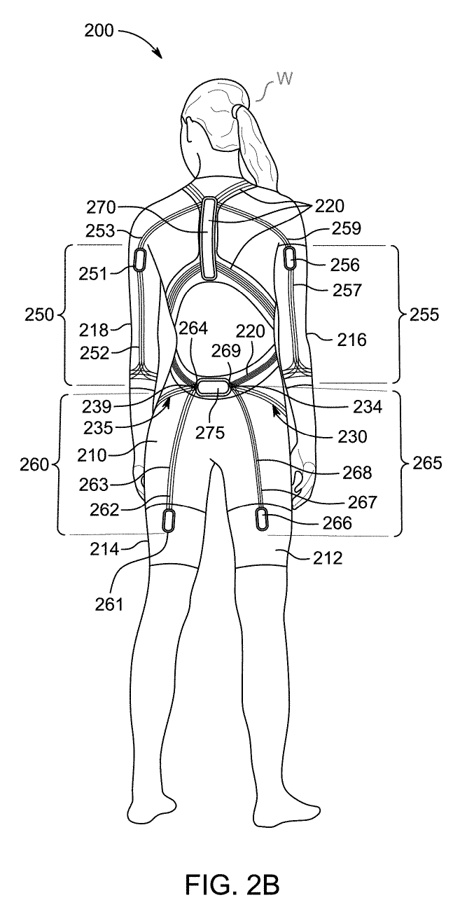

FIGS. 2A and 2B show front and back views of an illustrative exosuit 200 according to an embodiment. Exosuit 200 may embody some or all of the base layer, stability layer, power layer, sensor and controls layer, a covering layer, and user interface/user experience (UI/UX) layer, as discussed above. In addition, exosuit 200 may represent one of many different specification implementations of the exosuit shown in FIGS. 1A-1F. Exosuit 200 can include a base layer 210 with thigh LDMs 212 and 214, arm LDMs 216 and 218, and upper torso LDM 220. Thigh LDMs 212 and 214 may wrap around the thigh regions of the wearer, and arm LDMs 216 and 218 may wrap around the arm regions (e.g., including the elbow) of the wearer. Upper torso LDM 220 may wrap around the torso and neck of the wearer as shown. In particular, LDM 220 may cross near the abdomen, abut the sacrum, cover a portion of the back, and extend around the neck.

Exosuit 200 can include flexor PLSs 230 and 235 that may be secured to thigh LDMs 212 and 214 and upper torso LDM 220. Flexor PLSs 230 and 235 may provide leg muscle extensor movements. Flexor PLS 230 may include a flexdrive subsystem 231, a twisted string 232, and power/communication lines 233. Flexdrive subsystem 231 may include a motor, sensors, a battery, communications circuitry, and/or control circuitry. Twisted string 232 may be attached to flexdrive subsystem 231 and an attachment point 234 on LDM 220. Power/communications lines 233 may convey control signals and/or power to flexdrive subsystem 231. Flexor PLS 235 may include a flexdrive subsystem 236, a twisted string 237, and power/communication lines 238. Twisted string 237 may be attached to flexdrive subsystem 236 and an attachment point 239.

Exosuit 200 can include flexor PLSs 240 and 245 and extensor PLSs 250 and 255 that may be secured to LDMs 216, 218, and 220 (e.g., as shown). Flexor PLSs 240 and 245 may provide arm muscle flexor movements, and extensor PLSs 250 and 255 may provide arm muscle extensor movements. Flexor PLS 240 may include a flexdrive subsystem 241, a twisted string 242, and power/communication lines 243. Twisted string 242 may be attached to flexdrive subsystem 241 and an attachment point 244. Power/communication lines 243 may be coupled to a power and communications module 270. Flexor PLS 245 may include a flexdrive subsystem 246, a twisted string 247, and power/communication lines 248. Twisted string 247 may be attached to flexdrive subsystem 246 and an attachment point 249. Power/communication lines 248 may be coupled to power and communications module 270. Extensor PLS 250 may include a flexdrive subsystem 251, a twisted string 252, and power/communication lines 253. Twisted string 252 may be attached to flexdrive subsystem 251 and an attachment point 254. Power/communication lines 253 may be coupled to power and communications module 270. Extensor PLS 250 may include a flexdrive subsystem 256, a twisted string 257, and power/communication lines 258. Twisted string 256 may be attached to flexdrive subsystem 256 and an attachment point 259. Power/communication lines 258 may be coupled to power and communications module 270.

Exosuit 200 can include extensor PLSs 260 and 265 that may be secured to thigh LDMs 212 and 214 and LDM 220. Extensor PLSs 260 and 265 may provide leg muscle flexor movements. Extensor PLS 260 may include a flexdrive subsystem 261, a twisted string 262, and power/communication lines 263. Twisted string 262 may be attached to flexdrive subsystem 261 and an attachment point 264. Power/communication lines 263 may be coupled to a power and communications module 275. Flexor PLS 266 may include a flexdrive subsystem 266, a twisted string 267, and power/communication lines 268. Twisted string 267 may be attached to flexdrive subsystem 266 and an attachment point 269. Power/communication lines 263 may be coupled to power and communications module 275.

Exosuit 200 may be designed to assist, resist, and align movements being performed by the user of the suit. Exosuit 200 may include many sensors in various locations to provide data that may be used by control circuitry to determine and instruct or otherwise provide such movements. These sensors may be located anywhere on base layer 210 and may be electrically coupled to power and communications lines (e.g., 233, 238, 243, 248, 253, 258, 263, 268, and/or other lines). The sensors may provide absolute position data, relative position data, accelerometer data, gyroscopic data, inertial moment data, strain gauge data, resistance data, and/or any other suitable data.

Exosuit 200 may include a user interface 280 that may enable the user to control the exosuit. For example, user interface 280 can include several buttons or a touch screen interface and/or any other suitable user interface features. User interface 280 may also include a microphone to receive user spoken commands. User interface 280 may also include a speaker that can be used to playback voice recordings. Other user interface elements, including, but not limited to, buzzers (e.g., vibrating elements), may be strategically positioned around exosuit 200.

Exosuit 200 can include any suitable communications circuitry, such as that which may be contained in power and communications modules 270 or 275, to communicate directly with a user device (e.g., a smartphone) or with the user device via a central sever. The user may use the user device to select one or more movements he or she would like to perform, and upon selection of the one or more movements, exosuit 200 can then assist, resist, or align movement. The user device or exosuit 200 may provide real-time alignment guidance as to the user's performance of the movement, and exosuit 200 may provide resistance, alignment, or assistance to the movement.

An exosuit can be operated by electronic controllers that may be disposed on or within the exosuit and/or that may be in wireless or wired communication with the exosuit. The electronic controllers can be configured in a variety of ways to operate the exosuit and to enable functions of the exosuit. The electronic controllers can access and execute computer-readable programs that may be stored in elements of the exosuit and/or in other systems that may be in direct or indirect communications with the exosuit. The computer-readable programs can describe methods for operating the exosuit or can describe other operations relating to an exosuit or to a wearer of an exosuit.

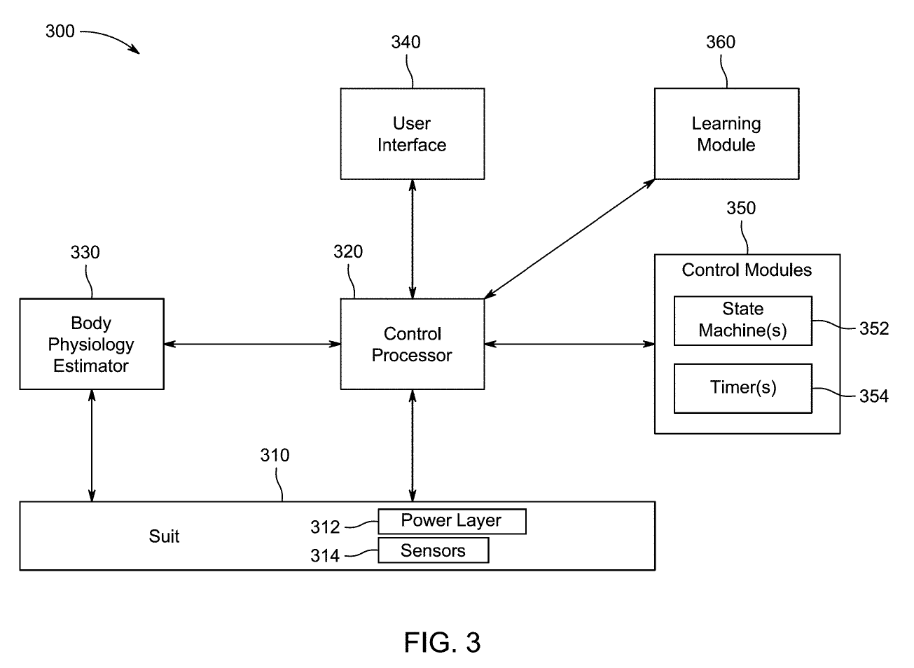

FIG. 3 shows an illustrative symbiosis exosuit system 300 according to an embodiment. The symbiosis may enable the exosuit to serve as an autonomous exosuit nervous system that may mimic or emulate the nervous system of a living organism, such as a human being. A nervous system may be responsible for basic life functions (e.g., breathing, converting food into energy, and maintaining muscle balance) that may be performed automatically without requiring conscious thought or input. The autonomous exosuit nervous system may enable the exosuit to automatically provide assistance to the user when and where the user needs it without requiring intervention by the user. Exosuit system 300 can do this by tracking the user's body physiology and automatically controlling the suit to provide the anticipated or appropriate support and/or assistance. For example, if a user has been standing for a prolonged period of time, one or more of the muscles being used to help the user stand may begin to tire, and as a result, the user's body may exhibit signs of fatigue. Exosuit 300 can observe this muscle fatigue (e.g., due to observed physiological signs) and can automatically cause exosuit 300 to engage the appropriate power layers to compensate for the muscle fatigue.

Symbiosis of exosuit 300 may be expressed in different autonomy levels, where each autonomy level may represent a degree to which physiological factors may be observed and a degree to which suit assistance or movement actions may be performed based on the observed physiological factors. For example, the symbiosis levels can range from a zero level of autonomy to an absolute full level of autonomy, with one or more intermediate levels of autonomy therebetween. As metaphorical example, autonomous cars operate according to different levels, where each level represents a different ability for the car to self-drive. The symbiosis levels of exosuit operation can be stratified in a similar manner In a zero level of autonomy, exosuit 300 may not monitor for any physiological cues, nor automatically engage any suit assistance or movement actions. Thus, in a zero level, the user may be required to provide user input to instruct the suit to perform a desired movement or assistance. In an absolute full level of autonomy, exosuit 300 may be able to observe and accurately analyze the observed physiological data (e.g., with 99% accuracy or more) and automatically execute the suit assistance or movement actions (e.g., in a way expressly desired by the user). Thus, in the absolute full level, the exosuit may seamlessly serve as an extension of the user's nervous system by automatically determining what the user needs and providing it.

The one or more intermediate levels of autonomy may provide different observable physiological results that may be accurate but may not represent the absolute nature of the absolute full level of autonomy. For example, an intermediate level may represent that the exosuit is fully capable of autonomously performing certain actions (e.g., sit to stand) but not others. A corollary to this is ABS braking, where the ABS braking system automatically figures out how best to stop the vehicle without requiring the user to pump the brakes or engage in any other activity other than stepping on the brake pedal. In the exosuit context, the exosuit may know when the user wishes to stand from a sitting position, the exosuit may know when the user wishes to perform the movement and engages the appropriate power layer segments to assist in the movement. The intermediate levels may also exist while the exosuit may be learning about its user. Each user is different, and the physiological responses may therefore be different and particular to each user. Therefore, the ability to discern the physiological cues and the assistance and movements made in response thereto may endure a learning curve before the suit is able to operate at the absolute full level.

FIG. 3 shows that exosuit system 300 can include a suit 310, a control processor 320, a body physiology estimator 330, a user interface 340, one or more control modules 350, and a learning module 360. Suit 310 can be any suitable exosuit (e.g., exosuit 200) and can include, among other things, a power layer 312 and one or more sensors 314. Control processor 320 may process instructions, pass data, and/or control the suit. Control processor 320 may be communicatively coupled in any suitable manner(s) to suit 310, body physiology estimator 330, user interface 340, control modules 350, and learning module 360. Control processor 320 may provide signals to suit 310 to control, for example, operation of power layer 312.

Body physiology estimator 330 may receive data inputs from one or more sensors 314, control processor 320, and/or any other components if desired. Estimator 330 may be operative to analyze the data to ascertain the physiology of the user. Estimator 330 may apply data analytics and statistics to the data to resolve physiological afflictions or conditions of the user's body. For example, estimator 330 can determine whether the user is sitting, standing, leaning, laying down, laying down on a side, walking, running, jumping, performing exercise movements, playing sports, reaching, holding an object or objects, or performing any other static or active physiological event. The results may be provided to control module(s) 350, for example, via control processor 320.

Sensors 314 can include one or more of any suitable accelerometer, gyroscope, magnetometer, altimeter sensor, electrocardiography (EKG) sensor, and/or any other suitable sensor. Sensors 314 may be integrated anywhere within the exosuit, though certain locations may be more preferred than others. For example, a sensor can be placed near the waist, upper body, shoes, thigh, arms, wrists, or head. In some embodiments, sensors can be embedded onto any equipment or device being used by the user. In some embodiments, the sensors can be contained external to the exosuit. For example, if worn on the wrist or arm of a worker, the device can be embedded into a watch, wrist band, elbow sleeve, or arm band. A second device may be used and clipped on the waist, on the pelvis, or slipped into a pocket in the garment, embedded into the garment itself, back-brace, belt, hard hat, protective glasses, or other personal protective equipment the worker may be wearing. The device can also be an adhesive patch worn on the skin. Other form factors can also clip onto the shoe or embedded into a pair of socks or the shoe itself

Control modules 350 can include one or more various state machines 352 and/or timers 354 that may be operative to control operation of suit 310 based on outputs supplied by estimator 330, inputs received via user interface 340, and/or signals provided by control processor 320. Multiple state machines 352 may control operation of the suit. For example, a master state machine may be supported by multiple slave state machines. The slave state machines may be executed in response to a call from the master state machine. In addition, the slave state machines may execute specific assistance functions or movements. For example, each of a sit-to-stand assistance movement, stand-to sit movement, stretch movement, standing movement, walking movement, running movement, jumping movement, crouch movement, specific exercise movement, or any other movement may have its own slave state machine to control suit operation.

Learning module 360 may be operative to learn preferences, peculiarities, or other unique features of a particular user and feedback the learnings to body physiology estimator 330 and/or control module(s) 350 and/or control processor 320. In some embodiments, learning module 360 may use data analytics to learn about the user. For example, learning module 360 may learn that a particular user walks with a particular gait and cadence. The gait and cadence learnings can be used to modify state machines 352 that may control walking for that user. In another embodiment, learning module 360 may incorporate user feedback received via user interface 340. For example, a user may go through an initial setup process whereby the user may be instructed to perform a battery of movements and provide responses thereto so that state machines 352 and timers 354 and/or any other components of system 300 may be set to operate in accordance with the preferences of the user.

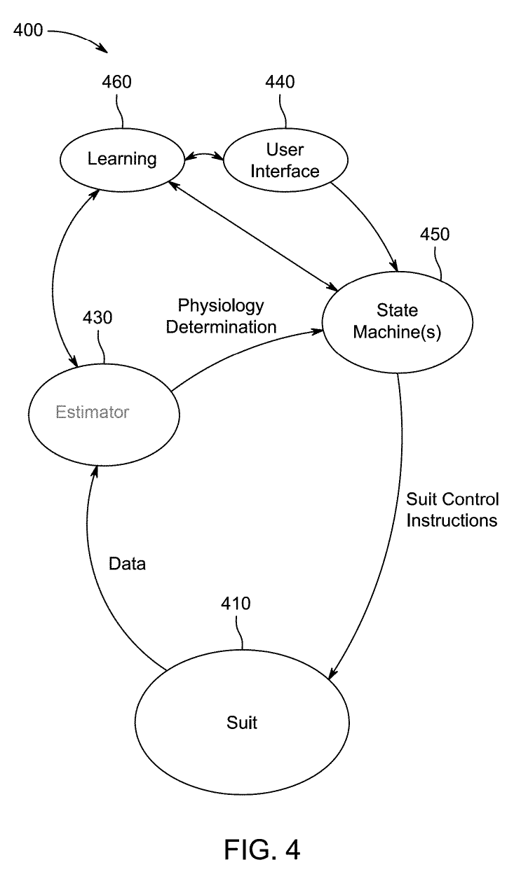

FIG. 4 shows an illustrative process 400 for implementing an exosuit system (e.g., symbiosis exosuit system 300) according to an embodiment. Process 400 may include a suit 410, an estimator 430, a user interface 440, and one or more state machines 450. Process 400 can be represented by a continuous feedback loop in which data may be supplied from suit 410 to estimator 430, which may provide a physiology determination to state machines 450, which may use the determination to generate suit control instructions that may be provided to suit 410. User inputs received via user interface 440 may provide user specified controls that can instruct state machines 450 to execute a particular movement. The autonomous exosuit nervous system may be implemented through the continuous feedback loop. The continuous feedback loop may enable the autonomous exosuit nervous system to provide rapid response and control of exosuit 410. For example, if the user is sitting down, estimator 430 can determine that the sitting position is the current physiological determination. Assume that the user reaches for something on a table. Such a movement may result in a movement that appears to be a sit-to-stand. In response to this movement, estimator 430 may register it as the start of a sit-to-stand physiological determination and instruct state machines 450 to initiate a sit-to-stand movement. This way, regardless of whether the user actually stands or sits back down, suit 410 may be primed and ready to immediately perform the assistance movement. Further assume that the user sits back down (e.g., after having grabbed the item on the table). In response to initiation of the sit down movement, estimator 430 can make this determination as it is happening and instruct state machines 450 to cease the sit-to-stand operation. Thus, the continuous feedback loop may provide real-time assessment and instantaneous suit controls in response to the user's immediate physiological needs, and not after.

In some embodiments, estimator 430 may be able to determine that the user is attempting to reach something on the table while also performing the motion that includes at least the start of a sit to stand movement. Estimator 430 may be able to correlate the reaching motion with the sit-to-stand motion and decide that the user does not actually need to stand, but may require an appropriate amount of assist to reach the item. In this particular situation, state machine 450 may activate a power layer segment (e.g., a particular one of the hip extensors) to provide the user with the reach assistance.

Learning 460 can receive and provide data to estimator 430, user interface 440, and state machines 450. Learning 460 may be leveraged to update state machines 450 and/or estimator 430.

Embodiments discussed herein refer to using exosuits to monitor worker safety and performance. That is, some applications of exosuits may be used in industrial applications in which workers don and use the exosuit to perform their duties. For example, in one embodiment, workers may use exosuits to perform heavy lifting tasks or to operate heavy equipment. The exosuit can monitor the workers as they perform their duties. The monitoring can enable injury detection or injury prevention. For example, the sensor data may show that the worker is exhibiting signs of fatigue or improper form in movement, both of which may lead to injury. The exosuit can provide additional assistance to compensate for the fatigue or improper form, or can provide feedback to inform the worker of the same. In other embodiments, the exosuit may be used to monitor worker productivity. If desired, an aggregate of work productivity data may be collected to assess worker productivity.

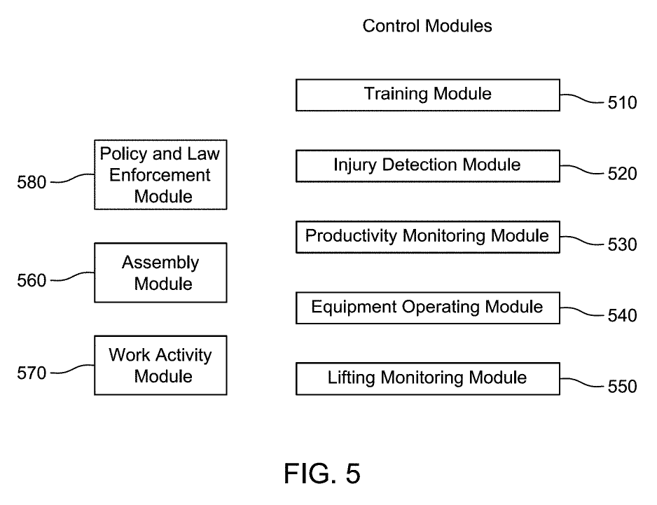

FIG. 5 shows an illustrative diagram of different control modules that may be implemented by an exosuit according to an embodiment. For example, the control modules of FIG. 5 may be implemented in control module 350 of FIG. 3. FIG. 5 can include a training module 510, an injury detection module 520, a productivity monitoring module 530, an equipment operating module 540, a lifting module 550, an assembly module 560, a work activity module 570, and/or a policy and law enforcement module 580. Other modules may be added as required. Each of the modules in FIG. 5 may be specifically configured to operate in connection with the suit by monitoring physiological movement of the user of the exosuit (e.g., via one or more sensors existing on the exosuit or otherwise of the exosuit system), controlling operation of the power layers of the exosuit, providing feedback to the user via the exosuit itself or by transmitting data to a device (e.g., a personal device) capable of providing the feedback, and/or the like.

Training module 510 may be accessed to provide on-the-job training of movements required to be performed by the worker during his or her shift. The training movements can include, for example, heavy lifting movements or ordinary lifting movements, heavy equipment operational movements, or conventional equipment operational movements, assembly movements, and/or any other suitable movement that can benefit from the use of an exosuit. The training module may include a step-by-by instruction course on how to use the exosuit in connection with the movement. The exosuit can be used to train individuals on how to properly operate the equipment, walking a user through all safety features, and/or providing real-time feedback on form and technique. Feedback can include the orientation and position of the user's forearm, upper-arm, shoulders, head, neck, pelvis, feet, and/or any other suitable anatomical feature. The training can also include providing real-time guidance on the proper orientation and use of the equipment itself. For example, the training can provide feedback that educates workers how to pick up objects properly with good bending techniques.

Injury detection module 520 may be used to detect injury in a worker or to detect circumstances or conditions that may lead to injury. Injury detection module 520 may have the ability to detect injuries that may occur on a relatively short term basis or a relatively long term basis. Relatively short term injuries may be those that occur as a result of a worker movement that causes an immediate step change in body physiology (e.g., such as pulled muscle or broken bone). Relatively long term injuries may be those that occur over a longer period of time (e.g., as a result of repetitive motion). Injury detection module 520 can detect job specific injuries. As a specific example, injury detection module 520 can detect injuries that are commonly found with construction workers. Construction workers often work with equipment that expose the worker to large forces on the human body. High intensity and high frequency vibrations from operating jack hammers, vibrating hand-held tools, saws, and other equipment can have significant long-term health impacts, such as Vibration Syndrome, vibration-induced white finger, and carpal tunnel syndrome. Vibration Syndrome can refer to a group of symptoms relating to the use of vibrating tools. Examples of Vibration Syndrome can include muscle weakness, fatigue, pain in the arms and shoulders, and blood-circulation failures in the fingers leading to an affliction known as white finger.

Injury detection module 520 may make injury assessments using many different approaches. For example, in one approach, injury detection can be done by analyzing the before and after walking gait of the user. There may be predominant differences in walking gait between a healthy person and the same injured person. For example, the exosuit can detect distinct indicators of injury, such as increased left and right gait asymmetry, increased pelvic rotation ranges, increased lateral sway, and sharp decreases in overall movement activities. Injury detection module 520 may characterize the injury event by detecting if a fall occurred or if a user was operating the equipment improperly or if the user was fatigued.

Injury detection module 520 may provide an alert if an injury has been detected. For example, if a fall has been detected and the worker is immobile, the exosuit can flash bright red and white LED lights and/or blast a message such as “Help, Man Down” over the exosuit's audio speaker. The exosuit can also triangulate and communicate the location of the fallen worker via GPS or Wi-Fi or by leveraging any suitable indoor tracking technologies. The exosuit may also trigger other lights or safety mechanisms the worker may be wearing or carrying. For example, the exosuit can cause a remote device, such as a connected head lamp, to flash pulses of light, or it can connect to the user's smartphone or handheld radio to broadcast a message to help rescue teams locate the person. Emergency alerts can be sent out to a team lead or safety lead if a worker is determined to be injured. For example, the closest teammate can also be alerted to assist the worker. Emergency Medical Service (EMS) can be configured to be notified automatically. In some embodiment, the user may cause an emergency alert to be sent out in the event that the exosuit system does not automatically detect the injury.

Productivity monitoring module 530 may monitor the productivity of one or more workers. For example, module 530 can quantify the amount of time the user is working on the job and how effective the worker is at doing his job. By monitoring the movement activities, vibrational intensities, and/or arm motion activities, for example, the exosuit can determine how productive an individual worker is compared to peers. Module 530 can identify workers who are over or underperforming. In some embodiments, over performing workers may be provided with micro bonuses (e.g., an extra vacation day) or overtime, and/or the consistent determination of over performance may be rewarded with a monetary year-end bonus. Module 530 can also monitor performance of the worker in completing tasks such as, for example, assembly speed and loading speed. Module 530 can ensure that the worker is conforming to mandatory work breaks, lunch breaks, and/or the like to ensure compliance with employment laws. Module 530 can be used as a motivational tool to encourage workers to increase their productivity. For example, module 530 may present competition like events among the workers to increase productivity and improve morale. Module 530 can determine which workers are at high risk of injury or are being particularly unproductive, and send an alert to a central system, worker or safety manager. In some embodiments, module 530 can automatically find another individual who is well rested who can take the place of the worker determined to be underperforming or at risk of injury to keep the job going.

Productivity monitoring module 530 may be able to coordinate collection of data from a multitude of sources and interpret that data to process worker productivity. For example, data may be collected from the exosuit, security cameras, badge stations, and other equipment. This way data across the entire workforce and workplace can be leveraged to optimize the productivity and safety of the workers. Predictive machine intelligence algorithms can be used to identify abnormal activities that correlate with injury and identify new red flag predictors that translate all the way back down to each individual. Machine intelligence models can also be used to predict worker productivity and identify opportunities for process and worker improvement. For example, a machine intelligence or algorithmic logic model can be used to measure the gradual decreases in worker productivity, increases in sedentary behavior, and predict worker attrition or injury.

A web dashboard can provide the safety manager or site manager with information on the productivity of the entire task force, identify which workers are at risk of injury from vibration overexposure, and which workers are under performing. Under performing workers can be identified by the lack of movement, steps, arm swings, high impact, or vibration exposure. The sensors can detect when a user is sitting or even lying down, thus, if a worker plans to sleep on the job, the system can alert the manager.

The data may be used to identify issues with equipment being used by the workers. For example, the data can identify which equipment transmits the largest vibration intensities and may therefore need repair or replacement. The number of fall detections or injuries can be geolocated to identify if there are certain high-risk areas in the construction site that needs to be addressed or give worker more caution.

Equipment operating module 540 may monitor a worker's use of equipment to ensure that the worker is using the equipment properly. The equipment can vary in size and complexity, including, for example, heavy equipment, such as construction equipment, mining equipment, and other “heavy” equipment and lite equipment, such as assembly line equipment, lawn and garden equipment, or other “lite” equipment. Module 540 can provide analysis of proper ergonomic biomechanics for equipment operation before, during, and after operation. The exosuit sensors can measure the orientation angles relative to the ground. Orientation angle along with equipment location and use can be used to approximate whether the user is operating the equipment ergonomically. For example, when the equipment is in operation, the orientation can change instantaneously due to vibrational forces being exerted on to the worker. Module 540 can quantify the orientation during operation.

Furthermore, while in operation, module 540 can calculate the changes in displacement (e.g., lateral, forward/backward, and up/down). While some displacement may be expected, namely in the vertical and forward/backward planes, significant changes in displacement can be an indicator of instability, especially in directions (e.g., lateral) where little displacement is expected. This may mean the worker is fatigued, has poor biomechanics during operation, or both. For example, when a user is about to operate equipment, such as a jackhammer, the arms should be held at specific angles relative to the ground to maintain proper control of the jackhammer throughout operation. If significant changes in arm orientation are detected, the worker may be losing control of the jack hammer, or may be losing grip of the handles, whereas if the orientation is stable, the worker has proper control. Furthermore, if there is significant lateral displacement detected, it may again show that the user is losing control or is using improper biomechanics. When events like this occur, module 540 can record and notify the worker immediately to address the issue and/or may automatically turn off the equipment to prevent injury.

Module 540 can prompt the worker to stretch or perform certain exercises before and after equipment operation to help mitigate and avoid injury. During such a prompt, module 540 can record and measure the stretches and exercises the worker actually did to help measure compliance.

Module 540 can leverage data obtained from sensors placed on the equipment in conjunction with or to the exclusion of sensors in the exosuit. The equipment sensors can, for example, calculate the orientation (and orientation variability) of the equipment during operation and quantify the displacement (and displacement variability). If the equipment is operated with improper orientation, or has unexpected changes in orientation, or significant displacements in a direction that should not occur, the device can send feedback to the worker or site managers. Module 540 can corroborate exosuit data with equipment data to determine whether the worker is using the equipment properly.

Lifting module 550 may control the exosuit to assist the worker in performing lifting operations. For example, the worker may be required to perform a series of lifting moves as part of his shift. The exosuit, in combination with lifting module 550, can assist the worker in performing those moves. For example, if the worker is required to lift and place object, the exosuit can assist in those movements. In some embodiments, the weight of the objects may be too heavy for the worker to lift without exosuit assistance.

Assembly module 560 may control the exosuit to assist the worker in performing tasks associated with assembly of an object. In one embodiment, the assembly can include construction of the object from start to finish. In another embodiment, the assembly can be a stage in an assembly line process.

Work activity module 570 may control the exosuit to assist the worker in performing any suitable worked related tasks. Module 570 may represent a catch all module for controlling the exosuit in any manner deemed suitable for the worker's job requirements.

Policy and law enforcement module 580 may be used to ensure that workers are complying with company policies and the law. For example, some companies may have policies that govern the safety and expectations of its workers. Workers wearing the exosuit can be monitored to ensure that those policies are followed. In addition, worker compensation laws, employment laws, and other laws or regulations (e.g., OSHA) promulgated by governing bodies require strict compliance. Exosuits can monitor the worker to ensure that the relevant laws and regulations are being abided.

In order for the control modules (e.g., modules shown in FIGS. 3 and 5) to perform their respective tasks, the control modules may require knowledge of the physiology or biomechanical movements of the worker wearing the exosuit. As indicated in FIG. 3, sensors 314 may provide movement data to body physiology estimator 330, which may analyze the data to extrapolate physiological or biomechanical movements of the worker. When the movements of the worker are known, control modules can use this information to provide worker safety monitoring, worker productivity monitoring, exosuit based worker assistance, and/or compliance monitoring according to various embodiments.

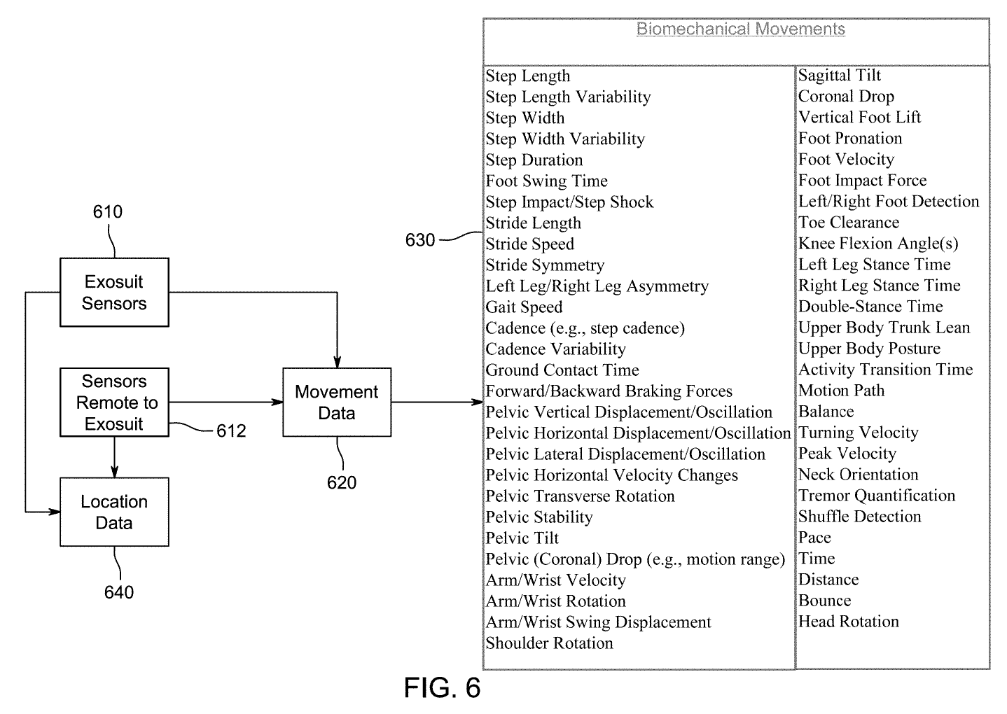

FIG. 6 shows an illustrative block diagram according to an embodiment. In particular, FIG. 6 shows that exosuit sensors 610 may provide movement data 620, which can be extrapolated to biomechanical movements 630. Sensors 612 remote to the exosuit may also provide movement data. For example, sensors remote to the exosuit may include sensors residing on a piece of equipment or environmental feature being used by or otherwise interacted with by the worker. An illustrative, and non-exhaustive, list of biomechanical movements 630 that may be sensed by any suitable sensor assembly(ies) of the system may be shown by listing of biomechanical movements 630 that can include, but is not limited to, step length, step length variability, step width, step width variability, step duration, foot swing time, step impact/step shock, stride length, stride speed, stride symmetry, left leg/right leg asymmetry, gait speed, cadence (e.g., step cadence), cadence variability, ground contact time, forward/backward braking forces, pelvic vertical displacement/oscillation, pelvic horizontal displacement/oscillation, pelvic lateral displacement/oscillation, pelvic horizontal velocity changes, pelvic transverse rotation, pelvic stability, pelvic tilt, pelvic (coronal) drop (e.g., motion range), arm/wrist velocity, arm/wrist rotation, arm/wrist swing displacement, shoulder rotation, sagittal tilt, coronal drop, vertical foot lift, foot pronation, foot velocity, foot impact force, left/right foot detection, toe clearance, knee flexion angle(s), left leg stance time, right leg stance time, double-stance time, upper body trunk lean, upper body posture, activity transition time, motion path, balance, turning velocity, peak velocity, neck orientation, tremor quantification, shuffle detection, pace, time, distance, bounce, head rotation, and/or the like. Biomechanical movements 630 can represent various components of any suitable generic body movements, such as, for example, walking, standing, running, squatting, lifting, throwing, and/or the like. Categorizing generic movements into granular biomechanical movements may provide a rich data set for accurately detecting and monitoring many or all aspects of the generic movement. Such data may enable the control modules to execute their programming with a high degree of accuracy and effectiveness. The movement data 620 may be used obtain other metrics, such as overall steps, energy expenditure, duration and intensity of exposures to vibrational activities, duration and biomechanics of proper operation of equipment, overall activity of hands, lack of sedentary behavior, and peak impact analysis.

The sensor data can be used to determine, for example, the time spent walking, running, sitting, standing, or lying down, the number of walking steps or running steps, the number of calories that were burned during any activity, the posture of a user during any of activity, and body stretches, range of motion arm and leg swings and various exercises, and/or the like. Additionally, while walking or miming, the sensor data can be used to quantify gait dynamics such as pelvic stability, range of motion in degrees of pelvic drop, tilt and rotation, the amount of vertical oscillation of the pelvis, forward/backward braking forces, step cadence (e.g., number of steps per minute), stride asymmetry, ground contact time, left step/right step, turning velocity, peak velocity, limp detection, and/or the like. The sensor data can be used to detect shock events and vibration events. The sensor data can be used to detect lifting characteristics, such as, for example, proper lifting from the knees, improper lifting from the lower back, twisting, and bending from the waist, and/or the like.

The sensor data may be used to validate whether a worker was injured on the job, measure worker productivity and identify if a worker is at risk of injury due to a newly detected limp, balance and sway characteristics in walking gait, and/or the like. Worker productivity is yet another metric that can be measured by a number of other activities including overall steps, energy expenditure duration and intensity of exposures to vibrational activities, duration and biomechanics of proper operation of equipment, overall activity of hands, lack of sedentary behavior, and peak impact analysis. For example, the sensor data may indicate a sudden abnormal walking gait from a worker with increased lateral sway and left/right leg stance time asymmetry—indicators of walk instability, potentially due to a sudden injury. In another case, the sensor data can indicate when a worker is carrying too much weight by detecting a bending motion followed by increased lateral sway and pelvic rotation.

FIG. 6 also shows that exosuit sensors 610 and sensors 612 remote to exosuit can provide location data 640. Location data 640 can indicate the location of the worker within the workplace, such as a particular room or section of a store, factory, work site, mine, and/or the like. As such, location data 640 may include GPS data, wireless signals (e.g., Wi-Fi, Bluetooth, ZigBee, etc.) for establishing location via triangulation, camera data that shows location of the user, transponder data, badge in/badge out data, or any other data that may disclose the location of the worker. Location data may be used by one or more of the control modules. For example, module 580 may use location data to determine whether the worker is taking a mandatory break. As another example, a module may use the location data to activate certain exosuit features based on the location of the worker.

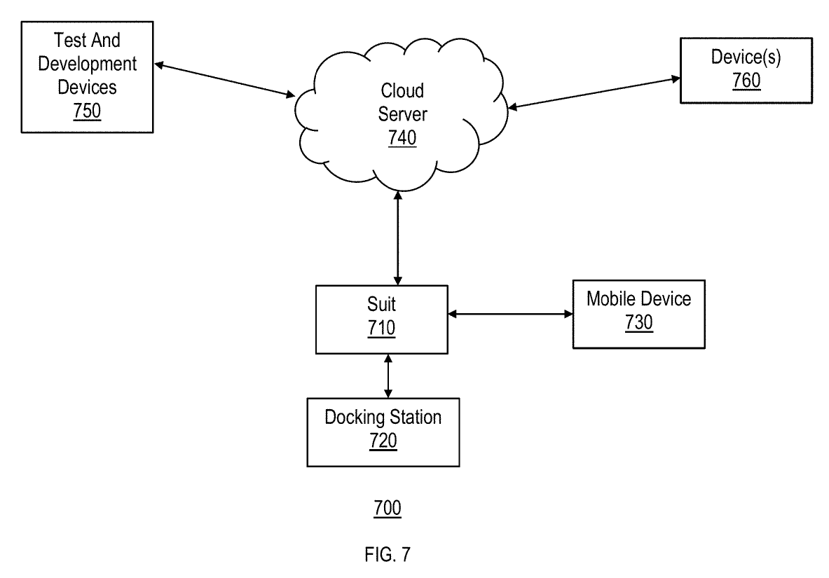

FIG. 7 shows an illustrative system 700 according to an embodiment. System 700 can include a suit 710, a docking station 720, a mobile device 730, a cloud server 740, test and development devices 750, and any other suitable devices 760. Suit 710 can include firmware that may be responsible for the user experience of the suit and/or for logging and tracking data. The firmware can be reprogrammed to provide new functional movements, gestures, sensors, and suit configurations. Suit 710 can log and transmit raw data (e.g., sensor data, motor data, algorithm outputs, etc.) to cloud server 740. Suit 710 can also log suit events, crashes, errors, suit health metrics, and other data to cloud server 740. Cloud server 740 may be a cloud-based platform that may manage personal user data and business intelligence, and may archive such data and/or share such data with any other suitable devices. The archived data may be used by test and development devices 750. Cloud server 740 may serve as a data layer that may provide highly customizable suits that may learn and adapt to user preferences and monitored uses. Cloud server 740 can handle near-time processing, long-term storage, permissions and authentication, and APIs, and/or sharing for personal user data. Test and development tools 750 may include tools for the development and testing of suits 710. Docking station 720 may be used to charge batteries and off load any data that may be stored on suit 710. Suit 710 may communicate with mobile devices 730 and/or devices 760 either directly or indirectly via cloud server 740. Devices 730 and 760 may run applications that may provide users and/or any suitable caretakers or other users (e.g., doctors, employers, etc.) with information related to use of their suits. The applications may display “dashboards” of information to the user or other appropriate entities.

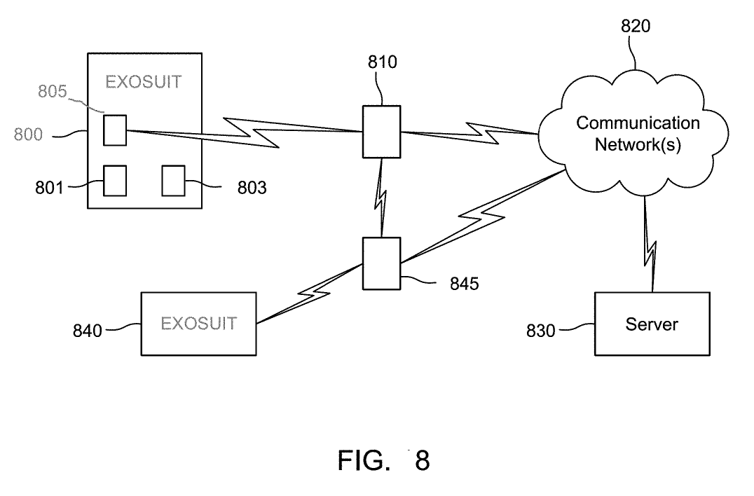

FIG. 8 illustrates a system with an example exosuit 800 that may include one or more actuators 801, one or more sensors 803, and at least one controller 805 that may be configured to operate elements of exosuit 800 (e.g., actuator(s) 801, sensor(s) 803, etc.) to enable functions of exosuit 800. Controller 805 may be configured to communicate wirelessly with a user interface 810 that may be configured to present information to a user (e.g., a wearer of exosuit 800) and to controller 805 of the flexible exosuit or to other systems. User interface 810 can be involved in controlling and/or accessing information from elements of exosuit 800. For example, an application being executed by user interface 810 can access data from sensors 803, calculate an operation (e.g., to apply dorsiflexion stretch) of actuators 801, and transmit the calculated operation to exosuit 800. User interface 810 can additionally be configured to enable other functions, such as, for example, user interface 810 can be configured to be used as a cellular telephone, a portable computer, an entertainment device, or to operate according to other applications.

User interface 810 can be configured to be removably mounted to exosuit 800 (e.g., by straps, magnets, Velcro, charging and/or data cables, etc.). Alternatively, user interface 810 can be configured as a part of exosuit 800 and not to be removed during normal operation. In some examples, a user interface can be incorporated as part of exosuit 800 (e.g., a touchscreen integrated into a sleeve of exosuit 800) and can be used to control and/or access information about exosuit 800 in addition to using user interface 810 to control and/or access information about exosuit 800. In some examples, controller 805 and/or any other elements of exosuit 800 may be configured to enable wireless or wired communication according to one or more standard protocols (e.g., Bluetooth, ZigBee, WiFi, LTE or other cellular standards, IRdA, Ethernet, etc.), such that a variety of systems and devices can be made to operate as user interface 810 when configured with complementary communications elements and computer-readable programs to enable such functionality.

Exosuit 800 can be configured as described in example embodiments herein or in other ways according to an application. Exosuit 800 can be operated to enable a variety of applications. Exosuit 800 can be operated to enhance the strength of a wearer by detecting motions of the wearer (e.g., using sensors 803) and responsively applying torques and/or forces to the body of the wearer (e.g., using actuators 801) to increase the forces the wearer is able to apply to his/her body and/or environment. Exosuit 800 can be operated to train a wearer to perform certain physical activities. For example, exosuit 800 can be operated to enable rehabilitative therapy of a wearer. Exosuit 800 can operate to amplify motions and/or forces produced by a wearer undergoing therapy in order to enable the wearer to successfully complete a program of rehabilitative therapy. Additionally or alternatively, exosuit 800 can be operated to prohibit disordered movements of the wearer and/or to use actuators 801 and/or other elements (e.g., haptic feedback elements) to indicate to the wearer a motion or action to perform and/or motions or actions that should not be performed or that should be terminated. Similarly, other programs of physical training (e.g., dancing, skating, other athletic activities, vocational training, etc.) can be enabled by operation of exosuit 800 to detect motions, torques, or forces generated by a wearer and/or to apply forces, torques, or other haptic feedback to the wearer. Other applications of exosuit 800 and/or user interface 810 are anticipated.

User interface 810 can additionally communicate with communications network(s) 820. For example, user interface 810 can include a WiFi radio, an LTE transceiver, or other cellular communications equipment, a wired modem, or some other elements to enable user interface 810 and exosuit 800 to communicate with the Internet. User interface 810 can communicate through communications network 820 with a server 830. Communication with server 830 can enable functions of user interface 810 and exosuit 800. In some examples, user interface 810 can upload telemetry data (e.g., location, configuration of elements 801 and/or 803 of exosuit 800, physiological data about a wearer of exosuit 800, etc.) to server 830.

In some examples, server 830 can be configured to control and/or access information from elements of exosuit 800 (e.g., actuator(s) 801, sensor(s) 803, etc.) to enable some application of exosuit 800. For example, server 830 can operate elements of exosuit 800 to move a wearer out of a dangerous situation if the wearer was injured, unconscious, or otherwise unable to move themselves and/or operate exosuit 800 and user interface 810 to move themselves out of the dangerous situation. Other applications of a server in communications with an exosuit are anticipated.

User interface 810 can be configured to communicate with a second user interface 845 in communication with and configured to operate a second flexible exosuit 840. Such communication can be direct (e.g., using radio transceivers or other elements to transmit and receive information over a direct wireless or wired link between user interface 810 and the second user interface 845). Additionally or alternatively, communication between user interface 810 and second user interface 845 can be facilitated by communications network(s) 820 and/or server 830 that may be configured to communicate with user interface 810 and second user interface 845 through communications network(s) 820.

Communication between user interface 810 and second user interface 845 can enable applications of exosuit 800 and second exosuit 840. In some examples, actions of exosuit 800 and second flexible exosuit 840 and/or of wearers of exosuit 800 and second exosuit 840 can be coordinated. For example, exosuit 800 and second exosuit 840 can be operated to coordinate the lifting of a heavy object by the wearers. The timing of the lift, and the degree of support provided by each of the wearers and/or exosuit 800 and second exosuit 840 can be controlled to increase the stability with which the heavy object was carried, to reduce the risk of injury of the wearers, or according to some other consideration. Coordination of actions of exosuit 800 and second exosuit 840 and/or of wearers thereof can include applying coordinated (e.g., in time, amplitude, or other properties) forces and/or torques to the wearers and/or elements of the environment of the wearers and/or applying haptic feedback (e.g., through actuators of the exosuits 800, 840, through dedicated haptic feedback elements, or through other methods) to the wearers to guide the wearers toward acting in a coordinated manner.

Coordinated operation of exosuit 800 and second exosuit 840 can be implemented in a variety of ways. In some examples, one exosuit (and the wearer thereof) can act as a master, providing commands or other information to the other exosuit such that operations of exosuit 800, 840 are coordinated. For example, exosuits 800, 840 can be operated to enable the wearers to dance (or to engage in some other athletic activity) in a coordinated manner. One of the exosuits can act as the “lead”, transmitting timing or other information about the actions performed by the “lead” wearer to the other exosuit, enabling coordinated dancing motions to be executed by the other wearer. In some examples, a first wearer of a first exosuit can act as a trainer, modeling motions or other physical activities that a second wearer of a second exosuit can learn to perform. The first exosuit can detect motions, torques, forces, or other physical activities executed by the first wearer and can send information related to the detected activities to the second exosuit. The second exosuit can then apply forces, torques, haptic feedback, or other information to the body of the second wearer to enable the second wearer to learn the motions or other physical activities modeled by the first wearer. In some examples, server 830 can send commands or other information to exosuits 800, 840 to enable coordinated operation of exosuits 800, 840.

Exosuit 800 can be operated to transmit and/or record information about the actions of a wearer, the environment of the wearer, or other information about a wearer of exosuit 800. In some examples, kinematics related to motions and actions of the wearer can be recorded and/or sent to server 830 (e.g., biokinematics (e.g., as mentioned with respect to FIG. 10). These data can be collected for medical, scientific, entertainment, social media, or other applications. The data can be used to operate a system. For example, exosuit 800 can be configured to transmit motions, forces, and/or torques generated by a user to a robotic system (e.g., a robotic arm, leg, torso, humanoid body, or some other robotic system) and the robotic system can be configured to mimic the activity of the wearer and/or to map the activity of the wearer into motions, forces, or torques of elements of the robotic system. In another example, the data can be used to operate a virtual avatar of the wearer, such that the motions of the avatar mirrored or were somehow related to the motions of the wearer. The virtual avatar can be instantiated in a virtual environment, presented to an individual or system with which the wearer is communicating, or configured and operated according to some other application.

Conversely, exosuit 800 can be operated to present haptic or other data to the wearer. In some examples, actuators 801 (e.g., twisted string actuators, exotendons, etc.) and/or haptic feedback elements (e.g., EPAM haptic elements) can be operated to apply and/or modulate forces applied to the body of the wearer to indicate mechanical or other information to the wearer. For example, the activation in a certain pattern of a haptic element of exosuit 800 disposed in a certain location of exosuit 800 can indicate that the wearer had received a call, email, or other communications. In another example, a robotic system can be operated using motions, forces, and/or torques generated by the wearer and transmitted to the robotic system by exosuit 800. Forces, moments, and other aspects of the environment and operation of the robotic system can be transmitted to exosuit 800 and presented (e.g., using actuators 801 or other haptic feedback elements) to the wearer to enable the wearer to experience force-feedback or other haptic sensations related to the wearer's operation of the robotic system. In another example, haptic data presented to a wearer can be generated by a virtual environment (e.g., an environment containing an avatar of the wearer that is being operated based on motions or other data related to the wearer that is being detected by exosuit 800).

Note that exosuit 800 illustrated in FIG. 8 is only one example of an exosuit that can be operated by control electronics, software, or algorithms described herein. Control electronics, software, or algorithms as described herein can be configured to control flexible exosuits or other mechatronic and/or robotic system having more, fewer, or different actuators, sensors or other elements. Further, control electronics, software, or algorithms as described herein can be configured to control exosuits configured similarly to or differently from illustrated exosuit 800. Further, control electronics, software, or algorithms as described herein can be configured to control flexible exosuits having reconfigurable hardware (e.g., exosuits that are able to have actuators, sensors, or other elements added or removed) and/or to detect a current hardware configuration of the flexible exosuits using a variety of methods.

A controller of an exosuit and/or computer-readable programs executed by the controller can be configured to provide encapsulation of functions and/or components of the flexible exosuit. That is, some elements of the controller (e.g., subroutines, drivers, services, daemons, functions, etc.) can be configured to operate specific elements of the exosuit (e.g., a twisted string actuator, a haptic feedback element, etc.) and to allow other elements of the controller (e.g., other programs) to operate the specific elements and/or to provide abstracted access to the specific elements (e.g., to translate a command to orient an actuator in a commanded direction into a set of commands sufficient to orient the actuator in the commanded direction). This encapsulation can allow a variety of services, drivers, daemons, or other computer-readable programs to be developed for a variety of applications of a flexible exosuits. Further, by providing encapsulation of functions of a flexible exosuit in a generic, accessible manner (e.g., by specifying and implementing an application programming interface (API) or other interface standard), computer-readable programs can be created to interface with the generic, encapsulated functions such that the computer-readable programs can enable operating modes or functions for a variety of differently-configured exosuit, rather than for a single type or model of flexible exosuit. For example, a virtual avatar communications program can access information about the posture of a wearer of a flexible exosuit by accessing a standard exosuit API. Differently-configured exosuits can include different sensors, actuators, and other elements, but can provide posture information in the same format according to the API. Other functions and features of a flexible exosuit, or other robotic, exoskeletal, assistive, haptic, or other mechatronic system, can be encapsulated by APIs or according to some other standardized computer access and control interface scheme.

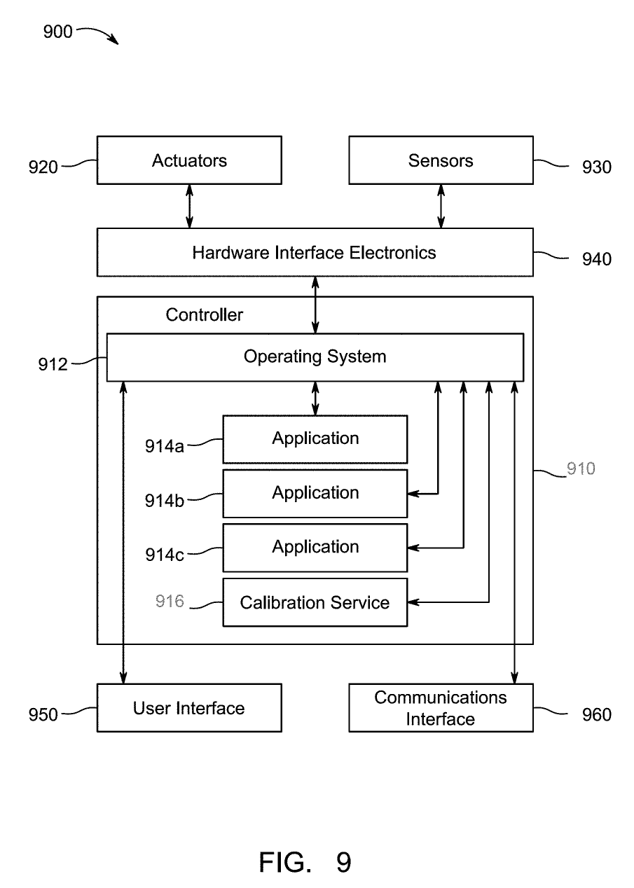

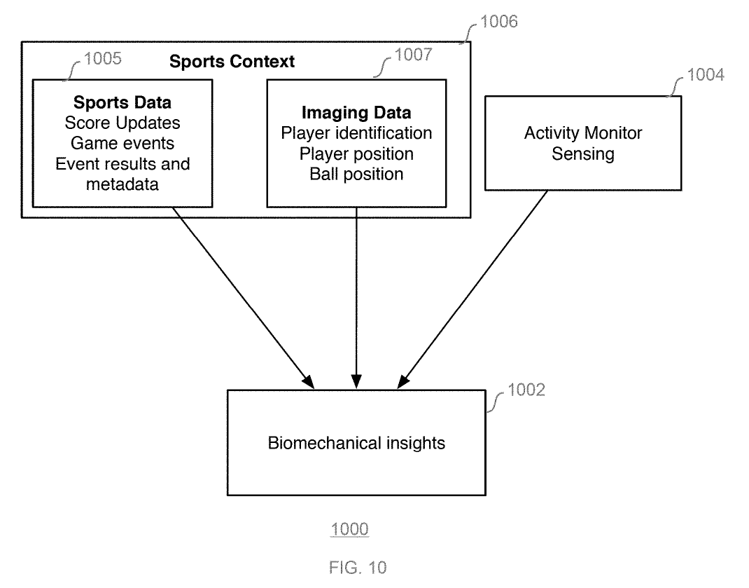

FIG. 9 is a schematic illustrating elements of an exosuit 900 and a hierarchy of control that may be used for operating exosuit 900. Flexible exosuit 900 may include one or more actuators 920 and one or more sensors 930 configured to apply forces and/or torques to and detect one or more properties of, respectively, exosuit 900, a wearer of exosuit 900, and/or the environment of the wearer. Exosuit 900 additionally may include a controller 910 that may be configured to operate actuators 920 and sensors 930 by using hardware interface electronics 940. Hardware interface electronics 940 may include electronics configured to interface signals from and to controller 910 with signals that may be used to operate actuators 920 and sensors 930. For example, actuators 920 can include exotendons, and hardware interface electronics 940 can include high-voltage generators, high-voltage switches, and/or high-voltage capacitance meters to clutch and un-clutch the exotendons and to report the length of the exotendons. Hardware interface electronics 940 can include voltage regulators, high voltage generators, amplifiers, current detectors, encoders, magnetometers, switches, controlled-current sources, DACs, ADCs, feedback controllers, brushless motor controllers, and/or other electronic and mechatronic elements.

Controller 910 additionally may be configured to operate a user interface 950 that may be configured to present information to a user and/or wearer of exosuit 900 and a communications interface 960 that may be configured to facilitate the transfer of information between controller 910 and some other system (e.g., by transmitting a wireless signal). Additionally or alternatively, user interface 950 can be part of a separate system that may be configured to transmit and receive user interface information to/from controller 910 using communications interface 960 (e.g., user interface 950 can be part of a cellphone).

Controller 910 may be configured to execute computer-readable programs describing functions of flexible exosuit 900. Among the computer-readable programs executed by controller 910 may be an operating system 912, one or more applications 914a, 914b, and 914c, and/or a calibration service 916. Operating system 912 may be configured to manage hardware resources of controller 910 (e.g., I/O ports, registers, timers, interrupts, peripherals, memory management units, serial and/or parallel communications units, etc.) and, by extension, may be configured to manage the hardware resources of exosuit 900. Operating system 912 may be the only computer-readable program executed by controller 910 that has direct access to hardware interface electronics 940 and, by extension, actuators 920 and sensors 930 of exosuit 900.

Applications 914a, 914b, and/or 914c may be computer-readable programs that describe some function, functions, operating mode, or operating modes of exosuit 900. For example, application 914a can describe a process for transmitting information about the wearer's posture to update a virtual avatar of the wearer that may include accessing information on a wearer's posture from operating system 912, maintaining communications with a remote system using communications interface 960, formatting the posture information, and/or sending the posture information to a remote system. Calibration service 916 may be a computer-readable program describing processes to store parameters describing properties of wearers, actuators 920, and/or sensors 930 of exosuit 900, to update those parameters based on operation of actuators 920 and/or sensors 930 when a wearer is using exosuit 900, to make the parameters available to operating system 912 and/or applications 914a, 914b, and/or 914c, and/or other functions relating to the parameters. Note that applications 914a, 914b, and/or 914c and/or calibration service 916 are intended as only some examples of computer-readable programs that can be run by operating system 912 of controller 910 to enable functions or operating modes of exosuit 900.

Operating system 912 can provide for low-level control and maintenance of the hardware (e.g., 920, 930, 940, etc.). In some examples, operating system 912 and/or hardware interface electronics 940 can detect information about exosuit 900, the wearer, and/or the wearer's environment from one or more sensors 930 at a constant specified rate. Operating system 912 can generate an estimate of one or more states or properties of exosuit 900 or components thereof using the detected information. Operating system 912 can update the generated estimate at the same rate as the constant specified rate or at a lower rate. The generated estimate can be generated from the detected information using a filter to remove noise, generate an estimate of an indirectly-detected property, or according to some other application. For example, operating system 912 can generate the estimate from the detected information using a Kalman filter to remove noise and to generate an estimate of a single directly or indirectly measured property of exosuit 900, the wearer, and/or the wearer's environment using more than one sensor. In some examples, operating system 912 can determine information about the wearer and/or exosuit 900 based on detected information from multiple points in time. For example, operating system 912 can determine an eversion stretch and dorsiflexion stretch.

In some examples, operating system 912 and/or hardware interface electronics 940 can operate and/or provide services related to operation of one or more actuators 920. That is, in a case where operation of actuators 920 requires the generation of control signals over a period of time, knowledge about a state or states of actuators 920, or other considerations, operating system 912 and/or hardware interface electronics 940 can translate simple commands to operate actuators 920 (e.g., a command to generate a specified level of force using a twisted string actuator (TSA) of actuators 920) into the complex and/or state-based commands to hardware interface electronics 940 and/or actuators 920 that may be necessary to effect the simple command (e.g., a sequence of currents applied to windings of a motor of a TSA, based on a starting position of a rotor determined and stored by operating system 912, a relative position of the motor detected using an encoder, and/or a force generated by the TSA detected using a load cell, etc.).

In some examples, operating system 912 can further encapsulate the operation of exosuit 900, such as by translating a system-level simple command (e.g., a commanded level of force tension applied to a footplate) into commands for multiple actuators, according to the configuration of exosuit 900. This encapsulation can enable the creation of general-purpose applications that can effect a function of an exosuit (e.g., allowing a wearer of the exosuit to stretch his foot) without being configured to operate a specific model or type of exosuit (e.g., by being configured to generate a simple force production profile that operating system 912 and hardware interface electronics 940 can translate into actuator commands that may be sufficient to cause actuators 920 to apply the commanded force production profile to the footplate).

Operating system 912 can act as a standard, multi-purpose platform to enable the use of a variety of exosuits having a variety of different hardware configurations to enable a variety of mechatronic, biomedical, human interface, training, rehabilitative, communications, and other applications. Operating system 912 can make sensors 930, actuators 920, or other elements or functions of exosuit 900 available to remote systems in communication with exosuit 900 (e.g., using communications interface 960) and/or a variety of applications, daemons, services, or other computer-readable programs being executed by operating system 912. Operating system 912 can make the actuators, sensors, or other elements or functions available in a standard way (e.g., through an API, communications protocol, or other programmatic interface), such that applications, daemons, services, or other computer-readable programs can be created to be installed on, executed by, and operated to enable functions or operating modes of a variety of flexible exosuits having a variety of different configurations. The API, communications protocol, or other programmatic interface made available by operating system 912 can encapsulate, translate, or otherwise abstract the operation of exosuit 900 to enable the creation of such computer-readable programs that are able to operate to enable functions of a wide variety of differently-configured flexible exosuits.

Additionally or alternatively, operating system 912 can be configured to operate a modular flexible exosuit system (e.g., a flexible exosuit system wherein actuators, sensors, or other elements can be added or subtracted from a flexible exosuit to enable operating modes or functions of the flexible exosuit). In some examples, operating system 912 can determine the hardware configuration of exosuit 900 dynamically and can adjust the operation of exosuit 900 relative to the determined current hardware configuration of exosuit 900. This operation can be performed in a way that was “invisible” to computer-readable programs (e.g., application 914a, application 914b, and/or application 914c) that may be accessing the functionality of exosuit 900 through a standardized programmatic interface presented by operating system 912. For example, the computer-readable program can indicate to operating system 912, through the standardized programmatic interface, that a specified level of torque was to be applied to an ankle of a wearer of exosuit 900. Operating system 912 can responsively determine a pattern of operation of actuators 920, based on the determined hardware configuration of exosuit 900, that may be sufficient to apply the specified level of torque to the ankle of the wearer.

In some examples, operating system 912 and/or hardware interface electronics 940 can operate actuators 920 to ensure that exosuit 900 does not operate to directly cause the wearer to be injured and/or elements of exosuit 900 to be damaged. In some examples, this can include not operating actuators 920 to apply forces and/or torques to the body of the wearer that exceeded some maximum threshold. This can be implemented as a watchdog process or some other computer-readable program that can be configured (e.g., when executed by controller 910) to monitor the forces being applied by actuators 920 (e.g., by monitoring commands sent to actuators 920 and/or monitoring measurements of forces or other properties detected using sensors 930) and to disable and/or change the operation of actuators 920 to prevent injury of the wearer. Additionally or alternatively, hardware interface electronics 940 can be configured to include circuitry to prevent excessive forces and/or torques from being applied to the wearer (e.g., by channeling to a comparator the output of a load cell that may be configured to measure the force generated by a TSA, and configuring the comparator to cut the power to the motor of the TSA when the force exceeded a specified level).

In some examples, operating actuators 920 to ensure that exosuit 900 does not damage itself can include a watchdog process or circuitry configured to prevent over-current, over-load, over-rotation, and/or other situations or conditions from occurring that can result in damage to elements of exosuit 900. For example, hardware interface electronics 940 can include a metal oxide varistor, breaker, shunt diode, and/or any other suitable elements that may be configured to limit the voltage and/or current applied to a winding of a motor.

Note that the above functions described as being enabled by operating system 912 can additionally or alternatively be implemented by applications 914a, 914b, and/or 914c, services, drivers, daemons, or other computer-readable programs executed by the controller 910. The applications, drivers, services, daemons, or other computer-readable programs can have special security privileges or other properties to facilitate their use to enable the above functions.

Operating system 912 can encapsulate the functions of hardware interface electronics 940, actuators 920, and/or sensors 930 for use by other computer-readable programs (e.g., applications 914a, 914b, and/or 914c, calibration service 916, etc.), by the user (e.g., through user interface 950), and/or by some other system (e.g., a system configured to communicate with controller 910 through communications interface 960). The encapsulation of functions of exosuit 900 can take the form of application programming interfaces (APIs) (e.g., sets of function calls and procedures that an application running on controller 910 can use to access the functionality of elements of exosuit 900). In some examples, operating system 912 can make available a standard “exosuit API” to applications being executed by controller 910. Such an “exosuit API” may be configured to enable applications 914a, 914b, and/or 914c to access functions of exosuit 900 without requiring those application(s) to be configured to generate whatever complex, time-dependent signals may be necessary to operate elements of exosuit 900 (e.g., actuators 920, sensors 930, etc.).

An “exosuit API” can allow applications 914a, 914b, and/or 914c to send simple commands to operating system 912 (e.g., “begin storing mechanical energy from the ankle of the wearer when the foot of the wearer contacts the ground”) in such a manner that operating system 912 can interpret those commands and generate the command signals to hardware interface electronics 940 or other elements of exosuit 900 that are sufficient to effect the simple commands generated by applications 914a, 914b, and/or 914c (e.g., determining whether the foot of the wearer has contacted the ground based on information detected by sensors 930, responsively applying high voltage to an exotendon that crosses the user's ankle, etc.).

An “exosuit API” can be an industry standard (e.g., an ISO standard), a proprietary standard, an open-source standard, or otherwise made available to individuals that can then produce applications for exosuits. An “exosuit API” can allow applications, drivers, services, daemons, or other computer-readable programs to be created that are able to operate a variety of different types and configurations of exosuits by being configured to interface with the standard “exosuit API” that is implemented by the variety of different types and configurations of exosuits. Additionally or alternatively, an “exosuit API” can provide a standard encapsulation of individual exosuit-specific actuators (e.g., actuators that apply forces to specific body segments, where differently-configured exosuits may not include an actuator that applies forces to the same specific body segments) and can provide a standard interface for accessing information on the configuration of whatever exosuit is providing the “exosuit API”. An application or other program that accesses an “exosuit API” can access data about the configuration of the exosuit (e.g., locations and forces between body segments generated by actuators, specifications of actuators, locations and specifications of sensors, etc.) and can generate simple commands for individual actuators (e.g., generate a force of 30 newtons for 50 milliseconds) based on a model of the exosuit generated by the application and based on the information on the accessed data about the configuration of the exosuit. Additional or alternate functionality can be encapsulated by an “exosuit API” according to an application.

Applications 914a, 914b, and/or 914c can individually enable all or parts of the functions and operating modes of a flexible exosuit described herein. For example, an application can enable haptic control of a robotic system by transmitting postures, forces, torques, and other information about the activity of a wearer of exosuit 900 and by translating received forces and torques from the robotic system into haptic feedback applied to the wearer (e.g., forces and torques applied to the body of the wearer by actuators 920 and/or haptic feedback elements). In another example, an application can enable a wearer to locomote more efficiently by submitting commands to and receiving data from operating system 912 (e.g., through an API) such that actuators 920 of exosuit 900 may assist the movement of the user, extract negative work from phases of the wearer's locomotion, and inject the stored work to other phases of the wearer's locomotion, or other methods of operating exosuit 900. Applications can be installed on controller 910 and/or on a computer-readable storage medium included in exosuit 900 by a variety of methods. Applications can be installed from a removable computer-readable storage medium or from a system in communication with controller 910 through communications interface 960. In some examples, the applications can be installed from a web site, a repository of compiled or un-compiled programs on the Internet, an online store (e.g., Google Play, iTunes App Store, etc.), or some other source. Further, functions of the applications can be contingent upon controller 910 being in continuous or periodic communication with a remote system (e.g., to receive updates, authenticate the application, to provide information about current environmental conditions, etc.).

Exosuit 900 illustrated in FIG. 9 is intended as an illustrative example. Other configurations of flexible exosuits and of operating systems, kernels, applications, drivers, services, daemons, or other computer-readable programs are anticipated. For example, an operating system configured to operate an exosuit can include a real-time operating system component configured to generate low-level commands to operate elements of the exosuit and a non-real-time component to enable less time-sensitive functions, like a clock on a user interface, updating computer-readable programs stored in the exosuit, or other functions. An exosuit can include more than one controller; further, some of those controllers may be configured to execute real-time applications, operating systems, drivers, or other computer-readable programs (e.g., those controllers were configured to have very short interrupt servicing routines, very fast thread switching, or other properties and functions relating to latency-sensitive computations), while other controllers may be configured to enable less time-sensitive functions of a flexible exosuit. Additional configurations and operating modes of an exosuit are anticipated. Further, control systems configured as described herein can additionally or alternatively be configured to enable the operation of devices and systems other than exosuit. For example, control systems as described herein can be configured to operate robots, rigid exosuits or exoskeletons, assistive devices, prosthetics, and/or other mechatronic devices.

Control of actuators of an exosuit can be implemented in a variety of ways according to a variety of control schemes. Generally, one or more hardware and/or software controllers can receive information about the state of the flexible exosuit, a wearer of the exosuit, and/or the environment of the exosuit from sensors disposed on or within the exosuit and/or a remote system in communication with the exosuit. The one or more hardware and/or software controllers can then generate a control output that can be executed by actuators of the exosuit to affect a commanded state of the exosuit and/or to enable some other application at the suit and/or at a remote application or dashboard for the benefit of the wearer or any other suitable entity (e.g., caretaker, etc.). One or more software controllers can be implemented as part of an operating system, kernel, driver, application, service, daemon, or other computer-readable program executed by a processor included in the exosuit.

Any suitable exosuit or at least any suitable sensor system with one or more sensors, which may be worn or otherwise carried by a user for monitoring activity of the user or equipment that may be used by the user or a competitor or other entity in the user's environment, may be utilized in a system for merging sports data and biokinematic data.

Systems, methods, and media for merging of sports data and biokinematic data are provided, which may function to provide enhanced, detailed analytics detailing the movement properties of athletes during sporting activities. The sports data preferably provides contextual signals that in combination with the biokinematic data can generate detailed analysis of players, plays, games, teams, and seasons among other forms of analysis. The systems and methods and media may preferably use sports data in detecting a context, which may then be used in selecting kinematic data of a particular time window for a particular player or set of players, and then performing a biokinematic analysis based on the context.

The systems and methods and media may utilize basic sports data, including, but not limited to, sports statistics and contextual information around a game such as: the game clock or period (e.g., first half, second quarter, two outs, etc.); score of the game; game events (e.g., kickoff, field goal attempts, jump balls, etc.); and team or player roles such as if a player is on defense or offense or if a player is serving or receiving.

The systems and methods and media may utilize image based sports data that can be applied to various actions, including, but not limited to, identifying player position in the field; ball possession; high level context of actions such as player A is defending player B, or Player X has the ball, and/or the like.

The systems and methods and media may utilize activity motion detection, which may involve using on player or equipment sensing (e.g., any suitable exosuit or at least any suitable sensor system with one or more sensors that may be worn or otherwise carried by a user or equipment that may be used by the user or a competitor or other entity in the user's environment), and/or which can be used in measuring a variety of kinematic properties of motions. Activity motion detection can use single point or multipoint sensing.

As a first benefit, the systems and methods and media can enable the selection and proper execution of biokinematic processing based on the context of a game or sport. The systems and methods and media may address a potential challenge in analyzing biokinematic motion data by narrowing the motion analysis options.

As a second benefit, the systems and methods and media can enable synchronized analysis of multiple participants. The biomechanics of an athlete's motions and timing of those motions can be analyzed and compared.

As a third benefit, the systems and methods and media can be used in analyzing biokinematic driven elements of a player or team over an entire game or season. Such detailed awareness can result in nutritional, training, and health recommendations. For example, the performance of a player returning from an injury could be analytically compared to performance from before the injury to prevent further complications. The use of contextual data can enable a wider variety of actions to be characterized by an activity monitor. The biomechanics of how a player performs certain actions can be monitored during the course of a player's career or season. If a player suffers an injury, the biomechanical characterization from before can be used in understanding how the action was changed from a biomechanical perspective.

The systems and methods and media can be applied to a variety of sports and to a variety of applications or use cases within those sports. The systems and methods and media may preferably be integrated into a form of tracking and/or even mapping the calories burned during a game.

As another exemplary use case, the systems and methods and media can be used in tracking fatigue levels of players. The various actions of a player could be individually tracked using the system and method over the course of a game and then compared to determine changes resulting from fatigue. As mentioned before a set of different actions could be monitored from a biomechanical perspective. The system and method can build an analytical understanding of the biomechanical properties of a player's actions when not fatigued. The non-fatigued biomechanical characterization can be compared to a player's current biomechanical performance to detect when a player deviates from normal performance. Non-linear deviations or other patterns of change may be a signal of fatigue. For example, the shooting motion of a basketball player may gradually change during the game resulting from fatigue, which may be detected through the biokinematic detection. Fatigue detection can be used in training, making coaching decisions (e.g., determining when to make a substitution), and for other suitable applications.

As another exemplary use case, the systems and methods and media can be applied to detecting the reaction time of a first player relative to a second player. For example, when a defender is guarding an offensive player in a sport such as soccer, football, or basketball, the system and method may be used to automatically detect how long it takes a defender to respond to the offensive player to faking one direction and then driving another.

As another exemplary use case, the systems and methods and media can be used in detecting the direction and/or orientation of players in a game. The activity motion can include direction sensed through a magnetometer of an IMU. The direction data can be used in combination with the sports data and/or image data to understand the relative position and orientation of players, the ball, and other relevant fixtures of a game (e.g., goal).

As another exemplary use case, the systems and methods and media can be applied to coaching and training. The biokinematics of a player during a successful play could be compared to those of unsuccessful plays to promote improved performance. Similarly, team management around training, play time, substitutions, jump height per player, and other team management options can be partially driven through biokinematic insights.

The systems and methods and media can be used for analyzing and altering decisions in sports in a variety of perspectives. From a first perspective, the systems and methods and media can be used for historical analysis of trends over time. This could include looking at players over a whole career or analyzing trends of an entire league. From another perspective, the systems and methods and media can be used as a post-performance analysis where a recent game or particular play can be analyzed in detail. In yet another perspective, the systems and methods and media may be used for in-progress analysis offering real-time or near real-time insights. For example real-time alerts could be triggered to alert coaches to possibly injury or other problems.

As shown in FIG. 10, a system 1000 of an embodiment can include a game data system 1005 (e.g., for providing score updates, game events, event results and metadata, etc.), an imaging data system 1007 (e.g., for providing player identification, player position, ball position, etc.) and at least one activity monitor sensing system or device 1004 (e.g., an exosuit and/or any other suitable sensor or collection of sensors on a user and/or equipment thereof) that may be used by a participant of a sport. Game data system 1005 and imaging data system 1007 (e.g., together, which may provide a sports context system 1006) may function to provide contextual insight that may then be used with the activity monitor sensing device 1004 for use in extracting detailed biokinematic analysis at a biomechanical insights system 1002. Activity monitor sensing device 1004 can preferably be used in analyzing a variety of actions. The contextual insights can be applied in determining what processes to use and what data to perform the analysis on. In one variation, sports context system 1006 can be implemented with only game data system 1005 or imaging data system 1007.

The game data system can be from any suitable source. The game data system can be the event log for a game, which tracks what players were involved in what plays, events, and/or other generally tracked metrics of a game.

The imaging data system can include one or more imaging systems. The imaging data systems may be video cameras but may alternatively include other suitable imaging systems, such as depth field cameras, infrared imaging, and/or any suitable type of imaging system. Alternatively, the system can include image system data inputs. The imaging data system can provide player identification, player position on a field, and/or other suitable information.

The activity monitor sensing device may be an inertial measurement unit system. The activity monitor sensing device may track a single point or multiple points on the body. A variety of actions can be characterized so as to be analyzed through the activity monitor sensing device. The game data system's data and imaging data system's data may be used in determining the segmenting of data and type of action to be analyzed from the activity monitor sensing device data.

The activity monitor sensing device may additionally be worn by multiple participants of a sport. Preferably, each of the participants of a sport may have an associated activity monitor sensing device or multiple activity monitor sensing devices. Data from the activity monitor sensing device can be processed on the device. Alternatively, the data can be communicated to a central resource, where data from multiple participants can be processed.

A method can include determining (e.g., at biomechanical insights system 1002) an action context from sports data, receiving kinematic data from one or more activity monitor sensing devices, selecting kinematic data based on the context, and applying a kinematic data processing routine according to the determined context.

The systems and methods associated with system 1000 can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods associated with system 1000 can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

Systems, methods, and computer-readable media may be provided to manage biomechanical achievements of a user of a user subsystem (e.g., an exosuit or other suitable subsystem with user activity sensing capabilities, user activity actuating capabilities, and/or the like), by predicting or determining one or more biomechanical achievements of the user (e.g., using one or more trained models and/or comparison(s) to actual achievements of other users) and, then, based on the determined biomechanical achievement(s), managing a mode of operation of the user subsystem and/or of any other suitable subsystem. Any suitable biomechanical model(s) (e.g., neural network(s) and/or learning engine(s)) may be trained and utilized in conjunction with any suitable condition data (e.g., data that may be indicative of any suitable characteristics of a condition of the user (e.g., age of user, weight of user, height of user, health history of user, location of user, etc.) and/or data that may be indicative any of any suitable characteristics of a user activity or user behavior performed by the user when exposed to or experiencing such a condition (e.g., sensed activity data detected by the user subsystem indicative of an activity performed by the user in the condition) and/or data that may be indicative of any suitable characteristics of a planned event to happen to the user in the condition (e.g., information indicative of a procedure or operation or therapy or any other suitable event that is to happen to the user at a certain time)) in order to predict or otherwise determine at least one biomechanical achievement of any particular user for any particular condition (e.g., generally, at a particular time (e.g., after a particular planned event), and/or for performing a particular activity). Such a biomechanical achievement may be analyzed with respect to particular rules or requirements or regulations or thresholds in order to generate any suitable control data for controlling any suitable functionality of any suitable output assembly of the user subsystem or of any other suitable subsystem (e.g., for adjusting a user interface presentation to a user (e.g., to suggest an action and/or to provide an alert to the user) and/or for adjusting an output that may affect an actual achievement of the user for the environment (e.g., for adjusting support provided by an actuator of an exosuit, etc.)).

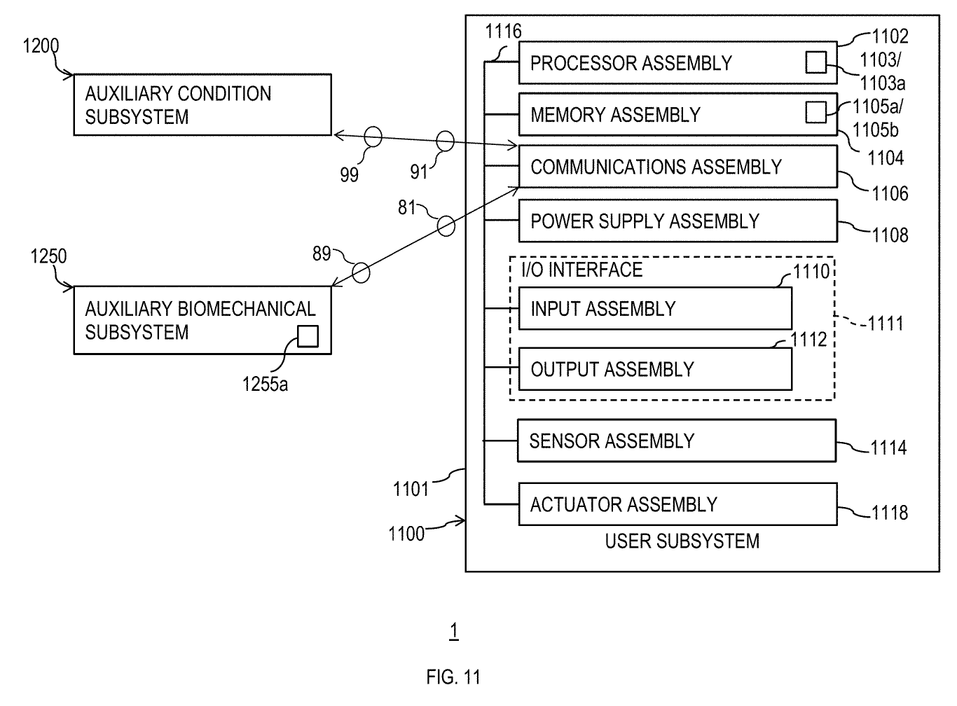

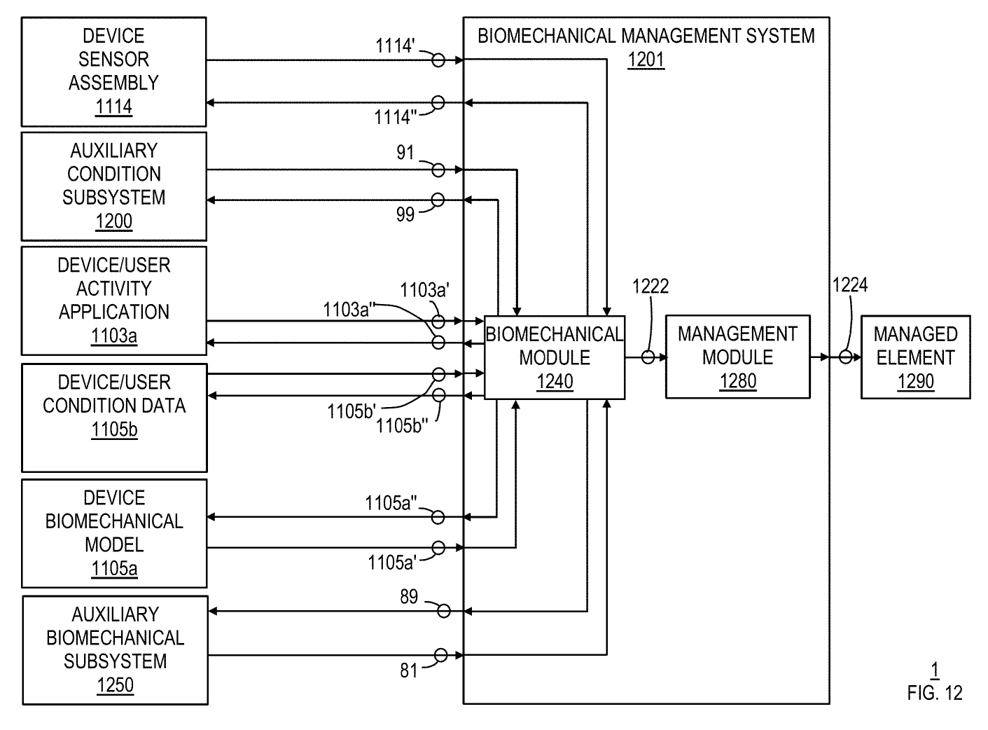

FIG. 11 is a schematic view of an illustrative system 1 that includes a user subsystem 1100 for managing biomechanical achievements in accordance with some embodiments. User subsystem 1100 can include, but is not limited to, a media player, media recorder, medical equipment, exercise equipment (e.g., treadmill), sporting equipment (e.g., tennis racquet, ball, etc.), appliance, transportation vehicle instrument, musical instrument, calculator, cellular telephone, any other wireless communication device, wearable device (e.g., a watch and/or an exosuit), personal digital assistant, remote control, pager, computer, or any combination thereof. In some embodiments, user subsystem 1100 may perform a single function (e.g., a device dedicated to sensing any suitable user activity or movement of a user wearing or carrying or otherwise interfacing with the user subsystem) and, in other embodiments, user subsystem 1100 may perform multiple functions (e.g., a device that senses any suitable user activity or movement of a user, supports and/or assists the user in performing certain activities (e.g., actuators for supporting and/or assisting a sit to stand activity or a walking activity or a lifting activity), plays music, and receives and transmits telephone calls). At least a portion (e.g., an activity sensing portion) of user subsystem 1100 may be provided by any portable, mobile, hand-held, or miniature user subsystem (e.g., electronic device) that may be configured to sense user activity of a user wherever the user travels. Some miniature electronic devices may have a form factor that is smaller than that of hand-held electronic devices. Illustrative miniature electronic devices can be integrated into various objects that may include, but are not limited to, watches, rings, necklaces, belts, accessories for belts, headsets, accessories for shoes, virtual reality devices, glasses, other wearable electronics, accessories for sporting clothing, sporting equipment, sporting accessories, accessories for fitness equipment, key chains, or any combination thereof. Alternatively, at least a portion (e.g., an activity sensing portion or any other portion (e.g., a power supply portion)) of user subsystem 1100 may not be portable at all, but may instead be generally stationary. Any suit or any suit system of FIGS. 1A-10 or any portion(s) thereof may be provided by user subsystem 1100.

As shown in FIG. 11, for example, user subsystem 1100 may include a processor assembly 1102, a memory assembly 1104, a communications assembly 1106, a power supply assembly 1108, an input assembly 1110, an output assembly 1112, a sensor assembly 1114, and an actuator assembly 1118. User subsystem 1100 may also include a bus 1116 that may provide one or more wired or wireless communication links or paths for transferring data and/or power to, from, or between various assemblies of user subsystem 1100. In some embodiments, one or more assemblies of user subsystem 1100 may be combined or omitted. Moreover, user subsystem 1100 may include any other suitable assemblies not combined or included in FIG. 11 and/or several instances of the assemblies shown in FIG. 11. For the sake of simplicity, only one of each of the assemblies is shown in FIG. 11.

Memory assembly 1104 may include one or more storage mediums, including for example, a hard-drive, flash memory, permanent memory such as read-only memory (“ROM”), semi-permanent memory such as random access memory (“RAM”), any other suitable type of storage assembly, or any combination thereof. Memory assembly 1104 may include cache memory, which may be one or more different types of memory used for temporarily storing data for electronic device applications. Memory assembly 1104 may be fixedly embedded within user subsystem 1100 or may be incorporated onto one or more suitable types of components that may be repeatedly inserted into and removed from user subsystem 1100 (e.g., a subscriber identity module (“SIM”) card or secure digital (“SD”) memory card). Memory assembly 1104 may store media data (e.g., music and image files), software (e.g., for implementing functions on subsystem 1100), firmware, preference information (e.g., media playback preferences), lifestyle information (e.g., food preferences), exercise information (e.g., information obtained by exercise monitoring applications), health information (e.g., information obtained by health monitoring applications and/or from health history records, etc.), sleep information (e.g., information obtained by sleep monitoring applications), mindfulness information (e.g., information obtained by mindfulness monitoring applications), wireless connection information (e.g., information that may enable subsystem 1100 to establish a wireless connection), contact information (e.g., telephone numbers and e-mail addresses), calendar information, any suitable device biomechanical model data of subsystem 1100 (e.g., as may be stored in any suitable device biomechanical model 1105a of memory assembly 1104), any suitable condition data 1105b of memory assembly 1104, any other suitable data, or any combination thereof.

Communications assembly 1106 may be provided to allow subsystem 1100 to communicate with one or more other user electronic devices or servers or subsystems or any other entities remote from subsystem 1100 (e.g., one or more of auxiliary subsystems 1200 and 1250 of system 1 of FIG. 11) using any suitable communications protocol(s). For example, communications assembly 1106 may support Wi-Fi™ (e.g., an 802.11 protocol), ZigBee™ (e.g., an 802.15.4 protocol), WiDi™, Ethernet, Bluetooth™, Bluetooth™ Low Energy (“BLE”), high frequency systems (e.g., 900 MHz, 2.4 GHz, and 5.6 GHz communication systems), infrared, transmission control protocol/internet protocol (“TCP/IP”) (e.g., any of the protocols used in each of the TCP/IP layers), Stream Control Transmission Protocol (“SCTP”), Dynamic Host Configuration Protocol (“DHCP”), hypertext transfer protocol (“HTTP”), BitTorrent™, file transfer protocol (“FTP”), real-time transport protocol (“RTP”), real-time streaming protocol (“RTSP”), real-time control protocol (“RTCP”), Remote Audio Output Protocol (“RAOP”), Real Data Transport Protocol™ (“RDTP”), User Datagram Protocol (“UDP”), secure shell protocol (“SSH”), wireless distribution system (“WDS”) bridging, any communications protocol that may be used by wireless and cellular telephones and personal e-mail devices (e.g., Global System for Mobile Communications (“GSM”), GSM plus Enhanced Data rates for GSM Evolution (“EDGE”), Code Division Multiple Access (“CDMA”), Orthogonal Frequency-Division Multiple Access (“OMMA”), high speed packet access (“HSPA”), multi-band, etc.), any communications protocol that may be used by a low power Wireless Personal Area Network (“6LoWPAN”) module, any other communications protocol, or any combination thereof. Communications assembly 1106 may also include or may be electrically coupled to any suitable transceiver circuitry that can enable subsystem 1100 to be communicatively coupled to another device (e.g., a server, host computer, scanner, accessory device, subsystem, etc.) and communicate data with that other device wirelessly or via a wired connection (e.g., using a connector port). Communications assembly 1106 (and/or sensor assembly 1114) may be configured to determine a geographical position of user subsystem 1100 and/or any suitable data that may be associated with that position. For example, communications assembly 1106 may utilize a global positioning system (“GPS”) or a regional or site-wide positioning system that may use cell tower positioning technology or Wi-Fi™ technology, or any suitable location-based service or real-time locating system, which may use a geo-fence for providing any suitable location-based data to subsystem 1100 (e.g., to determine a current geo-location of subsystem 1100 and/or any other suitable associated data (e.g., the current location is a gym, the current location is outside, the current location is your physical therapist's office, etc.)).

Power supply assembly 1108 may include any suitable circuitry for receiving and/or generating power, and for providing such power to one or more of the other assemblies of user subsystem 1100. For example, power supply assembly 1108 can be coupled to a power grid (e.g., when subsystem 1100 is not acting as a portable subsystem or when a battery of the subsystem is being charged at an electrical outlet with power generated by an electrical power plant). As another example, power supply assembly 1108 may be configured to generate power from a natural source (e.g., solar power using solar cells). As another example, power supply assembly 1108 can include one or more batteries for providing power (e.g., when subsystem 1100 is acting as a portable subsystem).

One or more input assemblies 110 may be provided to permit a user or subsystem environment to interact or interface with subsystem 1100. For example, input assembly 1110 can take a variety of forms, including, but not limited to, a touch pad, dial, click wheel, scroll wheel, touch screen, one or more buttons (e.g., a keyboard), mouse, joy stick, track ball, microphone, camera, scanner (e.g., a barcode scanner or any other suitable scanner that may obtain product identifying information from a code, such as a linear barcode, a matrix barcode (e.g., a quick response (“QR”) code), or the like), proximity sensor, light detector, temperature sensor, motion sensor, biometric sensor (e.g., a fingerprint reader or other feature (e.g., facial) recognition sensor, which may operate in conjunction with a feature-processing application that may be accessible to user subsystem 1100 for authenticating a user), line-in connector for data and/or power, and combinations thereof. Each input assembly 1110 can be configured to provide one or more dedicated control functions for making selections or issuing commands associated with operating subsystem 1100. Each input assembly 1110 may be positioned at any suitable location at least partially within a space defined by a housing 1101 of subsystem 1100 and/or at least partially on an external surface of housing 1101 of subsystem 1100.

User subsystem 1100 may also include one or more output assemblies 1112 that may present information (e.g., graphical, audible, and/or tactile information) to a user of subsystem 1100. For example, output assembly 1112 of user subsystem 1100 may take various forms, including, but not limited to, audio speakers, headphones, line-out connectors for data and/or power, visual displays (e.g., for transmitting data via visible light and/or via invisible light), infrared ports, flashes (e.g., light sources for providing artificial light for illuminating an environment of the subsystem), tactile/haptic outputs (e.g., rumblers, vibrators, etc.), and combinations thereof. As a specific example, user subsystem 1100 may include a display assembly output assembly as output assembly 1112, where such a display assembly output assembly may include any suitable type of display or interface for presenting visual data to a user with visible light.

It is noted that one or more input assemblies and one or more output assemblies may sometimes be referred to collectively herein as an input/output (“I/O”) assembly or I/O interface (e.g., input assembly 1110 and output assembly 1112 as I/O assembly or user interface assembly or I/O interface 1111). For example, input assembly 1110 and output assembly 1112 may sometimes be a single I/O interface 1111, such as a touch screen, that may receive input information through a user's touch of a display screen and that may also provide visual information to a user via that same display screen.

Sensor assembly 1114 may include any suitable sensor or any suitable combination of sensors that may be operative to detect any suitable movements or activities of user subsystem 1100 and/or of a user thereof and/or any other characteristics of subsystem 1100 and/or of its environment (e.g., physical activity or other characteristics of a user of subsystem 1100, light content of the subsystem environment, gas pollution content of the subsystem environment, temperature of the subsystem environment, altitude of the subsystem environment, incline or decline of the subsystem environment (e.g., incline or decline of a road on which the user is walking), etc.). Sensor assembly 1114 may include any suitable sensor(s) (e.g., any sensor of suit 100/170, any sensor of suit 200, any sensor 314 or otherwise of system 300, any sensor of suit 410, any sensor of the suit of FIG. 5, any sensor 610, any sensor 612, any sensor of suit 710, any sensor 803 or otherwise of FIG. 8, any sensor 930, any sensor of activity monitor sensing system 1004, etc.) that may detect any suitable activities or movements (e.g., biomechanical features) of the user and/or any other suitable characteristics of the user subsystem or of its environment, including, but not limited to, one or more of a GPS sensor, accelerometer, directional sensor (e.g., compass), gyroscope, magnetometer, compass, motion sensor, pedometer, passive infrared sensor, ultrasonic sensor, microwave sensor, a tomographic motion detector, a camera, a biometric sensor, a light sensor, a timer, or the like.

Sensor assembly 1114 may include any suitable sensor components or subassemblies for detecting any suitable movement of subsystem 1100 and/or of a user thereof. For example, sensor assembly 1114 may include one or more three-axis acceleration motion sensors (e.g., an accelerometer) that may be operative to detect linear acceleration in three directions (i.e., the x—or left/right direction, the y—or up/down direction, and the z—or forward/backward direction). As another example, sensor assembly 1114 may include one or more single-axis or two-axis acceleration motion sensors that may be operative to detect linear acceleration only along each of the x—or left/right direction and the y—or up/down direction, or along any other pair of directions. In some embodiments, sensor assembly 1114 may include an electrostatic capacitance (e.g., capacitance-coupling) accelerometer that may be based on silicon micro-machined micro electro-mechanical systems (“MEMS”) technology, including a heat-based MEMS type accelerometer, a piezoelectric type accelerometer, a piezo-resistance type accelerometer, and/or any other suitable accelerometer (e.g., which may provide a pedometer or other suitable function). Sensor assembly 1114 may be operative to directly or indirectly detect rotation, rotational movement, angular displacement, tilt, position, orientation, motion along a non-linear (e.g., arcuate) path, or any other non-linear motions. Additionally or alternatively, sensor assembly 1114 may include one or more angular rate, inertial, and/or gyro-motion sensors or gyroscopes for detecting rotational movement. For example, sensor assembly 1114 may include one or more rotating or vibrating elements, optical gyroscopes, vibrating gyroscopes, gas rate gyroscopes, ring gyroscopes, magnetometers (e.g., scalar or vector magnetometers), compasses, and/or the like. Any other suitable sensors may also or alternatively be provided by sensor assembly 1114 for detecting motion on subsystem 1100, such as any suitable pressure sensors, altimeters, or the like. Using sensor assembly 1114, user subsystem 1100 may be configured to determine a velocity, acceleration, orientation, and/or any other suitable motion attribute of user subsystem 1100.

Sensor assembly 1114 may include any suitable sensor components or subassemblies for detecting any suitable biomechanical data and/or health data and/or sleep data and/or mindfulness data and/or the like of a user of subsystem 1100. For example, sensor assembly 1114 may include any suitable biometric sensor that may include, but is not limited to, one or more health-related optical sensors, capacitive sensors, thermal sensors, electric field (“eField”) sensors, and/or ultrasound sensors, such as photoplethysmogram (“PPG”) sensors, electrocardiography (“ECG”) sensors, galvanic skin response (“GSR”) sensors, posture sensors, stress sensors, photoplethysmogram sensors, and/or the like. These sensors can generate data providing health-related information associated with the user. For example, PPG sensors can provide information regarding a user's respiratory rate, blood pressure, and/or oxygen saturation. ECG sensors can provide information regarding a user's heartbeats. GSR sensors can provide information regarding a user's skin moisture, which may be indicative of sweating and can prioritize a thermostat application to determine a user's body temperature. In some examples, each sensor can be a separate user subsystem, while, in other examples, any combination of two or more of the sensors can be included within a single user subsystem. For example, a gyroscope, accelerometer, photoplethysmogram, galvanic skin response sensor, and temperature sensor can be included within a wearable user subsystem, such as a smart watch, while a scale, blood pressure cuff, blood glucose monitor, SpO2 sensor, respiration sensor, posture sensor, stress sensor, and asthma inhaler can each be separate user subsystems. While specific examples are provided, it should be appreciated that other sensors can be used and other combinations of sensors can be combined into a single user subsystem. Using one or more of these sensors, one or more user subsystems 1100 can determine physiological characteristics of the user while performing a detected activity, such as a heart rate of a user associated with the detected activity, average body temperature of a user detected during the detected activity, any normal or abnormal physical qualities associated with the detected activity, or the like. In some examples, a GPS sensor or any other suitable location detection component(s) of subsystem 1100 can be used to determine a user's location (e.g., geo-location and/or address and/or location type (e.g., gym, physical therapist's office, bedroom, etc.) and movement, as well as a displacement of the user's motion. An accelerometer, directional sensor, and/or gyroscope can further generate activity data that can be used to determine whether a user of subsystem 1100 is engaging in an activity, is inactive, or is performing a gesture. Any suitable activity of a user may be tracked by sensor assembly 1114, including, but not limited to, steps taken, altitude inclined and/or declined, flights of stairs climbed, calories burned, distance walked, distance run, minutes of exercise performed and exercise quality, time of sleep and sleep quality, nutritional intake (e.g., foods ingested and their nutritional value), mindfulness activities and quantity and quality thereof (e.g., reading efficiency, data retention efficiency), any suitable work accomplishments of any suitable type (e.g., as may be sensed or logged by user input information indicative of such accomplishments), and/or the like. Subsystem 1100 can further include a timer that can be used, for example, to add time dimensions to various attributes of the detected physical activity, such as a duration of a user's physical activity or inactivity, time(s) of a day when the activity is detected or not detected, and/or the like.

Sensor assembly 1114 may include any suitable sensor components or subassemblies for detecting any suitable characteristics of any suitable feature of the lighting of the environment of subsystem 1100. For example, sensor assembly 1114 may include any suitable light sensor that may include, but is not limited to, one or more ambient visible light color sensors, illuminance ambient light level sensors, ultraviolet (“UV”) index and/or UV radiation ambient light sensors, and/or the like.

Sensor assembly 1114 may include any suitable sensor components or subassemblies for detecting any suitable characteristics of any suitable feature of the air quality of the environment of subsystem 1100. For example, sensor assembly 1114 may include any suitable air quality sensor that may include, but is not limited to, one or more ambient air flow or air velocity meters, ambient oxygen level sensors, volatile organic compound (“VOC”) sensors, ambient humidity sensors, ambient temperature sensors, and/or the like.

Sensor assembly 1114 may include any suitable sensor components or subassemblies for detecting any suitable characteristics of any suitable feature of the sound quality of the environment of subsystem 1100. For example, sensor assembly 1114 may include any suitable sound quality sensor that may include, but is not limited to, one or more microphones or the like that may determine the level of sound pollution or noise in the environment of subsystem 1100 (e.g., in decibels, etc.). Sensor assembly 1114 may also include any other suitable sensor for determining any other suitable characteristics about a user of subsystem 1100 and/or the environment of subsystem 1100 and/or any situation within which subsystem 1100 may be existing. For example, any suitable clock and/or position sensor(s) may be provided to determine the current time and/or time zone within which subsystem 1100 may be located.

One or more sensors of sensor assembly 1114 and/or any other component(s) of subsystem 1100 may be embedded in any suitable body or layer (e.g., housing 1101) of subsystem 1100, such as along a bottom surface that may be operative to contact a user, or can be positioned at any other desirable location. In some examples, different sensors can be placed in different locations inside or on the surfaces of subsystem 1100 (e.g., some located inside housing 1101 and some attached to an attachment mechanism (e.g., a wrist band coupled to a housing of a wearable device), or the like). In other examples, one or more sensors can be worn or otherwise interfaced by a user (e.g., as a sensor in or on equipment) separately as different parts of a single subsystem 1100 or as different user subsystems. In such cases, the sensors can be configured to communicate with subsystem 1100 using a wired and/or wireless technology (e.g., via communications assembly 1106). In some examples, sensors can be configured to communicate with each other and/or share data collected from one or more sensors. In some examples, subsystem 1100 can be waterproof such that the sensors can detect a user's activity in water.

Actuator assembly 1118 may include any suitable actuator or any suitable combination of actuators or other suitable component(s) that may be operative to support and/or assist a user of subsystem 1100 in any suitable manner for performing any suitable activities (e.g., actuators for supporting and/or assisting a sit to stand activity or a walking activity or a lifting activity). Actuator assembly 1118 may include any suitable actuator(s) or other suitable component(s) (e.g., any actuator of suit 100/170, any actuator of suit 200, any actuator of suit 310 or otherwise of system 300, any actuator of suit 410, any actuator of the suit of FIG. 5, any actuator of the system of FIG. 6, any actuator of suit 710, any actuator 801 or otherwise of FIG. 8, any actuator 920, any hardware interface electronics 940, any actuator of system 1000, etc.) that may support and/or assist any suitable activities or movements of the user (e.g., biomechanical features) and/or of the environment, including, but not limited to, one or more of a flexible linear actuator, twisted string actuator, flexdrive, exotendon, haptic feedback elements, stability components (e.g., elastic bands, springs, etc.), electrolaminate clutch, load distribution component, power layer segment, or the like.

System 1 may include one or more auxiliary condition subsystems 1200 that may include any suitable assemblies, such as assemblies that may be similar to one, some, or each of the assemblies of subsystem 1100. Subsystem 1200 may be configured to communicate any suitable auxiliary condition subsystem data 91 to subsystem 1100 (e.g., via a communications assembly of subsystem 1200 and communications assembly 1106 of subsystem 1100), such as automatically and/or in response to an auxiliary condition subsystem data request of data 99 that may be communicated from subsystem 1100 to auxiliary condition subsystem 1200. Such auxiliary condition subsystem data 91 may be any suitable condition attribute data that may be indicative of any suitable characteristic(s) of a condition of subsystem 1200 or an environment thereof as may be detected by auxiliary condition subsystem 1200 (e.g., as may be detected by any suitable input assembly and/or any suitable sensor assembly of auxiliary condition subsystem 1200) and/or any suitable subsystem state data that may be indicative of the current state of any components/features of auxiliary condition subsystem 1200 (e.g., any state of any suitable output assembly and/or of any suitable application of auxiliary condition subsystem 1200) and/or any suitable subsystem functionality data that may be indicative of any suitable functionalities/capabilities of auxiliary condition subsystem 1200. In some embodiments, such communicated auxiliary condition subsystem data 91 may be indicative of any suitable characteristic of an environment of auxiliary condition subsystem 1200 that may be an environment shared by subsystem 1100. For example, subsystem 1200 may include any suitable sensor assembly with any suitable sensors that may be operative to determine any suitable characteristic of an environment of subsystem 1200, which may be positioned in an environment shared by subsystem 1100. As just one example, subsystem 1200 may include or may be in communication with a heating, ventilation, and air conditioning (“HVAC”) subsystem of an environment, and subsystem 1100 may be able to access any suitable HVAC data (e.g., any suitable auxiliary condition subsystem data 91) from auxiliary condition subsystem 1200 indicative of any suitable HVAC characteristics (e.g., temperature, humidity, air velocity, oxygen level, harmful gas level, etc.) of the environment, such as when subsystem 1100 is located within that environment. As just one other example, subsystem 1200 may include or may be in communication with a game data system of an environment (e.g., game data system 1005) and/or with an imaging data system (e.g., imaging data system 1007) and/or any suitable sporting equipment and/or fitness equipment and/or medical equipment, and subsystem 1100 may be able to access any suitable game data and/or imaging data (e.g., any suitable auxiliary condition subsystem data 91) from auxiliary condition subsystem 1200 indicative of any suitable game characteristics. As yet just one other example, subsystem 1200 may be provided by a health service (e.g., a subsystem operated by a doctor's office or physical therapist's office or the like) that may be operative to determine or access, store, and/or provide any suitable health data for any suitable user (e.g., age, height, weight, medical history (e.g., diagnoses, surgeries, ailments, conditions, diets, etc.) and/or for any suitable location or environment (e.g., incline of a surface being walked by a user, amount of weight being lifted by a user, altitude of environment of user, etc.). It is to be understood that auxiliary condition subsystem 1200 may be any suitable subsystem that may be operative to determine or generate and/or control and/or access any suitable condition data about a particular environment and/or user and share such data (e.g., as any suitable auxiliary condition subsystem data 91) with subsystem 1100 at any suitable time, such as to augment and/or enhance the sensing capabilities of sensor assembly 1114 of subsystem 1100. User subsystem 1100 may be operative to communicate any suitable data 99 from communications assembly 1106 to a communications assembly of auxiliary condition subsystem 1200 using any suitable communication protocol(s), where such data 99 may be any suitable request data for instructing subsystem 1200 to share data 91 and/or may be any suitable auxiliary condition subsystem control data that may be operative to adjust any physical system attributes of auxiliary condition subsystem 1200 (e.g., of any suitable output assembly of auxiliary condition subsystem 1200 (e.g., to increase the temperature of air output by an HVAC auxiliary environment subsystem 1200, to adjust the incline of a surface of subsystem 1200 being walked by a user, amount of weight of subsystem 1200 being lifted by a user, altitude of environment of user, etc.)).

Subsystem 1100 and a user thereof may be situated in various conditions at various times (e.g., user running outdoors on a high altitude and steep incline path at 11:00 AM, user walking indoors on a sea level altitude and flat track at 8:00 PM, user having a high blood pressure in January and a low blood pressure in June, user having not yet had arthroscopic surgery on Mar. 5, 2016 but having had arthroscopic surgery by Dr. Doe on Mar. 6, 2016, etc.). At any particular condition in which subsystem 1100 and a user thereof may be situated at a particular time, any or all condition characteristic information indicative of the particular condition at the particular time may be sensed by subsystem 1100 from any or all features and data sources of the environment (e.g., directly via sensor assembly 1114 of subsystem 1100 and/or via any suitable auxiliary condition subsystem(s) 1200 of the environment). Such condition characteristic information that may be sensed or otherwise received by subsystem 1100 for a particular condition at a particular time may be processed and/or stored by subsystem 1100 as at least a portion of condition behavior data or condition data 1105b alone or in conjunction with any suitable user behavior information or user activity information that may be provided by user U (e.g., by input assembly 1110) or otherwise detected by subsystem 1100 (e.g., by sensor assembly 1114) and that may be indicative of a user's behavior within and/or a user's reaction to the particular condition, for example, as at least another portion of condition data 1105b. Any suitable user behavior information (e.g., user activity information) for a user at a particular condition at a particular time may be detected in any suitable manner by subsystem 1100 (e.g., any suitable user-provided feedback information may be provided by user U to subsystem 1100 (e.g., via any suitable input assembly 1110 (e.g., typed via a keyboard or dictated via a user microphone, etc.) or detected via any suitable sensor assembly or otherwise of subsystem 1100 or a subsystem 1200 of the environment) that may be indicative of the user's biomechanical achievements or other suitable activities or movements in the particular condition at the particular time (e.g., a subjective user-provided description of the activity (e.g., “running” or “swimming” or “power walking”), a subjective user-provided preference for adjusting the condition in some way, and/or the like) and/or that may be indicative of the user's performance of any suitable activity in the particular condition at the particular time (e.g., any suitable exercise activity information, any suitable sleep information, any suitable mindfulness information, etc. (e.g., which may be indicative of the user's effectiveness or ability to perform an activity within the particular environment))). Such condition characteristic information that may be sensed or otherwise received by subsystem 1100 for a particular condition at a particular time, as well as such user behavior information that may be sensed or otherwise received by subsystem 1100 for the particular condition at the particular time, may together be processed and/or stored by subsystem 1100 as at least a portion of condition data 1105b (e.g., for tracking a user's subjective biomechanical achievement(s) for a particular condition at a particular time and/or a user's objective activity performance capability for a particular condition at a particular time).

Processor assembly 1102 of user subsystem 1100 may include any processing circuitry that may be operative to control the operations and performance of one or more assemblies of user subsystem 1100. For example, processor assembly 1102 may receive input signals from input assembly 1110 and/or drive output signals through output assembly 1112. As shown in FIG. 11, processor assembly 1102 may be used to run one or more applications, such as an application 1103. Application 1103 may include, but is not limited to, one or more operating system applications, firmware applications, media playback applications, media editing applications, calendar applications, state determination applications, biometric feature-processing applications, compass applications, health applications, mindfulness applications, sleep applications, thermometer applications, weather applications, thermal management applications, video game applications, biomechanical applications, device and/or user activity applications, or any other suitable applications. For example, processor assembly 1102 may load application 1103 as a user interface program to determine how instructions or data received via an input assembly 1110 and/or sensor assembly 1114 and/or any other assembly of subsystem 1100 (e.g., any suitable auxiliary condition subsystem data 99 that may be received by subsystem 1100 via communications assembly 1106) may manipulate the one or more ways in which information may be stored on subsystem 1100 and/or provided to a user via an output assembly 1112 and/or actuator assembly 1118 and/or provided to an auxiliary condition subsystem (e.g., to subsystem 1200 as auxiliary condition subsystem data 91 via communications assembly 1106). Application 1103 may be accessed by processor assembly 1102 from any suitable source, such as from memory assembly 1104 (e.g., via bus 1116) or from another remote device or server (e.g., from a subsystem 1200 and/or from a subsystem 1250 of system 1 via communications assembly 1106). Processor assembly 1102 may include a single processor or multiple processors. For example, processor assembly 1102 may include at least one “general purpose” microprocessor, a combination of general and special purpose microprocessors, instruction set processors, graphics processors, video processors, and/or related chips sets, and/or special purpose microprocessors. Processor assembly 1102 also may include on board memory for caching purposes.

One particular type of application available to processor assembly 1102 may be an activity application 1103a that may be operative to determine or predict a current or planned activity or event of subsystem 1100 and/or for a user thereof. Such an activity may be determined by activity application 1103a based on any suitable data accessible by activity application 1103a (e.g., from memory assembly 1104 and/or from any suitable remote entity (e.g., any suitable auxiliary condition subsystem data 91 from any suitable auxiliary subsystem 1200 via communications assembly 1106)), such as data from any suitable activity data source, including, but not limited to, a calendar application, a health application, a social media application, an exercise monitoring application, a sleep monitoring application, a mindfulness monitoring application, transaction information, wireless connection information, subscription information, contact information, pass (e.g., event/ticketing) information, current condition data 1105b, previous condition data 1105b, biomechanical model data of any suitable biomechanical model, and/or the like. For example, at a particular time, such an activity application 1103a may be operative to determine one or more potential or planned or predicted user activities for that particular time, such as exercise, walk, run, lift, swim, sleep, play tennis, practice soccer, endure physical therapy, undergo a particular surgery or procedure on a particular biomechanical feature of the user, and/or the like. Alternatively, such an activity application 1103a may request that a user indicate a planned activity (e.g., via a user interface assembly).

User subsystem 1100 may also be provided with any suitable housing 1101 that may at least partially enclose at least a portion of one or more of the assemblies of subsystem 1100 for protection from debris and other degrading forces external to subsystem 1100. In some embodiments, one or more of the assemblies may be provided within its own housing (e.g., input assembly 1110 may be an independent keyboard or mouse within its own housing that may wirelessly or through a wire communicate with processor assembly 1102, which may be provided within its own housing).

Processor assembly 1102 may load any suitable application 1103 as a background application program or a user-detectable application program in conjunction with any suitable biomechanical model to determine how any suitable input assembly data received via any suitable input assembly 1110 and/or any suitable sensor assembly data received via any suitable sensor assembly 1114 and/or any other suitable data received via any other suitable assembly of subsystem 1100 (e.g., any suitable auxiliary condition subsystem data 91 received from auxiliary condition subsystem 1200 via communications assembly 1106 of subsystem 1100 and/or any suitable planned activity data as may be determined by activity application 1103a of subsystem 1100) may be used to determine any suitable biomechanical achievement data (e.g., biomechanical achievement state data 1222 of FIG. 12) that may be used to control or manipulate at least one functionality of subsystem 1100 (e.g., a performance or mode of user subsystem 1100 that may be altered in a particular one of various ways (e.g., particular alerts or recommendations may be provided to a user via a user interface assembly and/or particular adjustments may be made by an output assembly or actuator assembly and/or the like)). Any suitable biomechanical model or any suitable combination of two or more biomechanical models may be used by subsystem 1100 in order to make any suitable biomechanical achievement determination for any particular condition of any particular user of subsystem 1100 at any particular time (e.g., any biomechanical model(s) may be used in conjunction with any suitable condition data 1105b (e.g., any suitable condition characteristic information and/or any suitable user behavior information that may be sensed or otherwise received by subsystem 1100) and/or in conjunction with any suitable planned activity (e.g., any suitable activity as may be determined by activity application 1103a) to provide any suitable biomechanical achievement data that may be indicative of any biomechanical achievement determination for the particular condition at the particular time). For example, a device biomechanical model 1105a may be maintained and updated on subsystem 1100 (e.g., in memory assembly 1104) using processing capabilities of processor assembly 1102. Additionally or alternatively, an auxiliary biomechanical model 1255a may be maintained and updated by any suitable auxiliary biomechanical subsystem 1250 that may include any suitable assemblies, such as assemblies that may be similar to one, some, or each of the assemblies of subsystem 1100. Auxiliary biomechanical subsystem 1250 may be configured to communicate any suitable auxiliary biomechanical subsystem data 81 to subsystem 1100 (e.g., via a communications assembly of subsystem 1250 and communications assembly 1106 of subsystem 1100), such as automatically and/or in response to an auxiliary biomechanical subsystem data request of data 89 that may be communicated from subsystem 1100 to auxiliary biomechanical subsystem 1250. Such auxiliary biomechanical subsystem data 81 may be any suitable portion or the entirety of auxiliary biomechanical model 1255a for use by subsystem 1100 (e.g., for use by an application 1103 instead of or in addition to (e.g., as a supplement to) device biomechanical model 1105a).

A biomechanical model may be developed and/or generated for use in evaluating and/or predicting a biomechanical achievement for a particular condition (e.g., at a particular time and/or with respect to one or more particular activities for a particular user or type of user). For example, a biomechanical model may be a learning engine for an experiencing entity (e.g., a particular user or a particular subset or type of user or all users generally), where the learning engine may be operative to use any suitable machine learning to use certain condition data (e.g., one or more various types or categories of condition category data, such as condition data (e.g., condition characteristic information and/or user behavior information) and/or planned activity data) for a particular condition (e.g., at a particular time and/or with respect to one or more planned activities for a particular user or user type) in order to predict, estimate, and/or otherwise generate any suitable biomechanical achievement data and/or any suitable biomechanical achievement determination that may be indicative of the biomechanical achievement that may be experienced by the experiencing entity for, of, in, and/or with the particular condition by the experiencing entity (e.g., a biomechanical achievement that may be achieved or carried out by the user for the condition). For example, the learning engine may include any suitable neural network (e.g., an artificial neural network) that may be initially configured, trained on one or more sets of scored condition data from any suitable experiencing entity(ies) (e.g., condition data with a known biomechanical achievement of a particular experiencing entity for, of, in, and/or with a particular condition), and then used to predict a biomechanical achievement or any other suitable biomechanical achievement determination based on another set of condition data.

A neural network or neuronal network or artificial neural network may be hardware-based. software-based, or any combination thereof, such as any suitable model (e.g., an analytical model, a computational model, an algorithmic logic model, a machine intelligence model, a machine learning model, a prediction model, a regression model (e.g., a linear regression model (e.g., a multi-variable linear regression model)), etc.), which, in some embodiments, may include one or more sets or matrices of weights (e.g., adaptive weights, which may be numerical parameters that may be tuned by one or more learning algorithms or training methods or other suitable processes) and/or may be capable of approximating one or more functions (e.g., non-linear functions or transfer functions) of its inputs. The weights may be connection strengths between neurons of the network, which may be activated during training and/or prediction. A neural network may generally be a system of interconnected neurons that can compute values from inputs and/or that may be capable of machine learning and/or pattern recognition (e.g., due to an adaptive nature). A neural network may use any suitable machine learning techniques to optimize a training process. The neural network may be used to estimate or approximate functions that can depend on a large number of inputs and that may be generally unknown. The neural network may generally be a system of interconnected “neurons” that may exchange messages between each other, where the connections may have numeric weights (e.g., initially configured with initial weight values) that can be tuned based on experience, making the neural network adaptive to inputs and capable of learning (e.g., learning pattern recognition). A suitable optimization or training process may be operative to modify a set of initially configured weights assigned to the output of one, some, or all neurons from the input(s) and/or hidden layer(s). A non-linear transfer function may be used to couple any two portions of any two layers of neurons, including an input layer, one or more hidden layers, and an output (e.g., an input to a hidden layer, a hidden layer to an output, etc.).

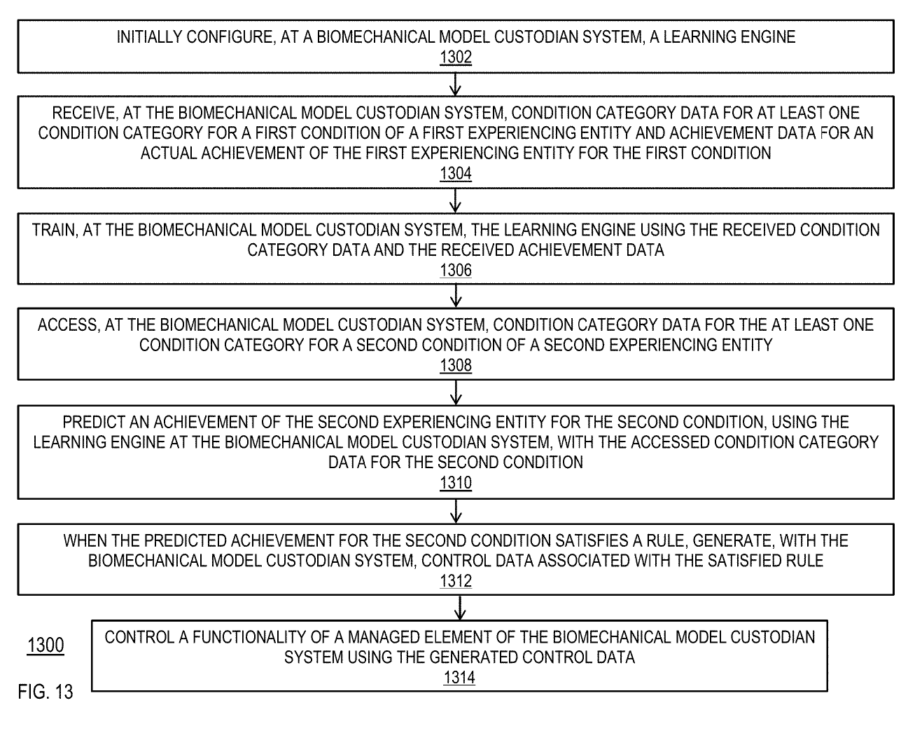

Different input neurons of the neural network may be associated with respective different types of condition categories and may be activated by condition category data of the respective condition categories (e.g., each possible category of condition characteristic information (e.g., temperature, altitude, oxygen level, air velocity, humidity, various gas levels (e.g., various VOC levels, pollen level, dust level, etc.), geo-location, location type, time of day, day of week, week of month, week of year, month of year, age of user, weight of user, height of user, health history of user, and/or the like), each possible category of user behavior information (e.g., sensed activity data detected by a user subsystem indicative of an activity performed by the user in the condition), and/or each possible category of planned activity (e.g., exercise, walk, swim, run, surgery by Dr. Doe, play soccer, etc.) may be associated with one or more particular respective input neurons of the neural network and condition category data for the particular condition category may be operative to activate the associated input neuron(s)). The weight assigned to the output of each neuron may be initially configured (e.g., at operation 1302 of process 1300 of FIG. 13) using any suitable determinations that may be made by a custodian or processor of the biomechanical model (e.g., subsystem 1100 and/or auxiliary biomechanical subsystem 1250) based on the data available to that custodian.

The initial configuring of the learning engine or biomechanical model for the experiencing entity (e.g., the initial weighting and arranging of neurons of a neural network of the learning engine) may be done using any suitable data accessible to a custodian of the biomechanical model (e.g., a manufacturer of subsystem 1100 or of a portion thereof (e.g., device biomechanical model 1105a), any suitable maintenance entity that manages auxiliary biomechanical subsystem 1250, and/or the like), such as data associated with the configuration of other learning engines of system 1 (e.g., learning engines or biomechanical models for similar experiencing entities), data associated with the experiencing entity (e.g., initial background data accessible by the model custodian about the experiencing entity's composition, background, interests, goals, past experiences, health history, and/or the like), data assumed or inferred by the model custodian using any suitable guidance, and/or the like. For example, a model custodian may be operative to capture any suitable initial background data about the experiencing entity in any suitable manner, which may be enabled by any suitable user interface provided to an appropriate subsystem or device accessible to one, some, or each experiencing entity (e.g., a model app or website). The model custodian may provide a data collection portal for enabling any suitable entity to provide initial background data for the experiencing entity. The data may be uploaded in bulk or manually entered in any suitable manner. In a particular embodiment where the experiencing entity is a particular user or a group of users, the following is a list of just some of the one or more potential types of data that may be collected by a model custodian (e.g., for use in initially configuring the model): sample questions for which answers may be collected may include, but are not limited to, questions related to an experiencing entity's evaluation of perceived biomechanical achievement capability with respect to a particular previously experienced condition (e.g., ability to run 5 miles), their preferred characteristics for an environment for an activity (e.g., preferred temperature and/or altitude and/or time of day (e.g., generally and/or for a particular planned activity and/or for a particular type of environment), ideal environment, and/or the like. 102101 A biomechanical model custodian may receive from the experiencing entity (e.g., at operation 1304 of process 1300 of FIG. 13) not only condition category data for at least one condition category for a particular condition that the experiencing entity is currently experiencing or has previously experienced but also a score or information indicative of a known or actual biomechanical achievement of the experiencing entity for, of, in, and/or with that particular condition experience. As just one example, an actual or known biomechanical achievement may be a known distance that the experiencing entity or a trusted GPS assembly may supply as an indication of a known distance that the experiencing entity ran while experiencing the condition (e.g., while generating sensed movement data of the experiencing entity running on a certain track with a certain incline). As just one other example, an actual or known biomechanical achievement may be additional performed user behavior or activity information that may be generated by sensing the experiencing entity perform a particular activity after a particular planned event of the condition (e.g., additional sensed movement data of the experiencing entity running on a certain date after an arthroscopic procedure on the experiencing entity's right knee by Dr. Doe (e.g., where the associated condition data may include other sensed movement data of the experiencing entity running on another certain date just prior to the arthroscopic procedure)). This may be enabled by any suitable user interface provided to any suitable experiencing entity by any suitable biomechanical model custodian (e.g., a user interface app or website that may be accessed by the experiencing entity). The biomechanical model custodian may provide a data collection portal for enabling any suitable entity to provide such actual or known biomechanical achievement data. The actual or known biomechanical achievement for the condition may be received and/or may be derived from the experiencing entity in any suitable manner. For example, a single questionnaire or survey may be provided by the model custodian (e.g., via any suitable user interface (e.g., I/O assembly 1111)) for deriving not only an experiencing entity's responses with respect to condition category data for a condition, but also an experiencing entity's actual biomechanical achievement for the condition. The model custodian may be configured to provide best practices and standardize much of the evaluation, which may be determined based on the experiencing entity's goals and/or objectives as captured before the condition may have been experienced. Additionally or alternatively, data indicative of an actual or known biomechanical achievement for the condition may be detected and/or received automatically from any suitable assemblies of the system (e.g., by any suitable sensor assembly(ies) (e.g., a GPS assembly, a biomechanical movement sensor assembly, etc.)) in any suitable manner.

A learning engine or biomechanical model for an experiencing entity may be trained (e.g., at operation 1306 of process 1300 of FIG. 13) using the received condition category data for the condition (e.g., as inputs of a neural network of the learning engine) and using the received actual biomechanical achievement for the condition (e.g., as an output of the neural network of the learning engine). Any suitable training methods or algorithms (e.g., learning algorithms) may be used to train the neural network of the learning engine, including, but not limited to, Back Propagation, Resilient Propagation, Genetic Algorithms, Simulated Annealing, Levenberg, Nelder-Meade, and/or the like. Such training methods may be used individually and/or in different combinations to get the best performance from a neural network. A loop (e.g., a receipt and train loop) of receiving condition category data and actual biomechanical achievement data for a condition and then training the biomechanical model using the received condition category data and actual biomechanical achievement data (e.g., a loop of operation 1304 and operation 1306 of process 1300 of FIG. 13) may be repeated any suitable number of times for the same experiencing entity and the same learning engine for more effectively training the learning engine for the experiencing entity, where the received condition category data and the received actual biomechanical achievement data received of different receipt and train loops may be for different conditions or for the same condition (e.g., at different times and/or with respect to different planned events or activity types) and/or may be received from the same source or from different sources of the experiencing entity (e.g., from different users of the experiencing entity) (e.g., a first receipt and train loop may include receiving condition category data and actual biomechanical achievement data from a first user with respect to that user's experience with a first condition, while a second receipt and train loop may include receiving condition category data and actual biomechanical achievement data from a second user with respect to that user's experience with the first condition (or with a condition substantially the same as the first condition but perhaps slightly different health data and/or surgery doctor and/or the like), while a third receipt and train loop may include receiving condition category data and actual biomechanical achievement data from a third user with respect to that user's experience with a condition for a planned arthroscopic knee surgery event, while a fourth receipt and train loop may include receiving condition category data and actual biomechanical achievement data from a fourth user with respect to that user's experience with a condition for a planned hip replacement surgery event, and/or the like), while the training of different receipt and train loops may be done for the same learning engine using whatever condition category data and actual biomechanical achievement data was received for the particular receipt and train loop. The number and/or type(s) of the one or more condition categories for which condition category data may be received for one receipt and train loop may be the same or different in any way(s) than the number and/or type(s) of the one or more condition categories for which condition category data may be received for a second receipt and train loop.

A biomechanical model custodian may access (e.g., at operation 1308 of process 1300 of FIG. 13) condition category data for at least one condition category for another condition (e.g., a condition that is different than any condition considered at any condition category data receipt of a receipt and train loop for training the learning engine for the experiencing entity (e.g., a condition differing in any suitable one or more ways (e.g., different user, different health history characteristic(s), different planned event characteristic(s), different user movements or activities being carried out and sensed, etc.). In some embodiments, this other condition may be a condition that has not been specifically experienced by any experiencing entity prior to use of the biomechanical model in an end user use case. Although, it is to be understood that this other condition may be any suitable condition. The condition category data for this other condition may be accessed from or otherwise provided by any suitable source(s) using any suitable methods (e.g., from one or more sensor assemblies and/or input assemblies of any suitable subsystem(s) 1100 and/or subsystem(s) 1200 that may be associated with the particular condition at the particular time) for use by the biomechanical model custodian (e.g., processor assembly 1102 of subsystem 1100 and/or auxiliary biomechanical subsystem 1250).

This other condition (e.g., condition of interest) may then be scored (e.g., at operation 1308 of process 1300 of FIG. 13) using the learning engine or biomechanical model for the experiencing entity with the condition category data accessed for such another condition. For example, the condition category data accessed for the condition of interest may be utilized as input(s) to the neural network of the learning engine (e.g., at operation 1310 of process 1300 of FIG. 13) similarly to how the condition category data accessed at a receipt portion of a receipt and train loop may be utilized as input(s) to the neural network of the learning engine at a training portion of the receipt and train loop, and such utilization of the learning engine with respect to the condition category data accessed for the condition of interest may result in the neural network providing an output indicative of a biomechanical achievement that may represent the learning engine's predicted or estimated biomechanical achievement to be derived from the condition of interest by the experiencing entity.

After a biomechanical achievement (e.g., any suitable biomechanical achievement data (e.g., biomechanical achievement state data 1222 of FIG. 12)) is realized for a condition of interest (e.g., for a current condition being experienced by an experiencing entity (e.g., for a particular time and/or for a particular planned event)), it may be determined (e.g., at operation 1312 of process 1300 of FIG. 13) whether the realized biomechanical achievement satisfies a particular rule of any suitable number of potential rules and, if so, the model custodian or any other suitable processor assembly or otherwise (e.g., of subsystem 1100 and/or of auxiliary biomechanical subsystem 1250) may generate any suitable control data (e.g., biomechanical mode data (e.g., biomechanical achievement mode data 1224 of system 1201 of FIG. 12)) that may be associated with that satisfied rule for controlling (e.g., at operation 1314 of process 1300 of FIG. 13) any suitable functionality of any suitable output assembly of subsystem 1100 or of auxiliary subsystem(s) 1200 and/or 1250 or otherwise (e.g., for adjusting a user interface presentation to a user (e.g., to provide a biomechanical achievement suggestion, a biomechanical achievement alert, etc.)) and/or for controlling any suitable functionality of any suitable output assembly of user subsystem 1100 or of auxiliary condition subsystem 1200 or otherwise (e.g., for adjusting support and/or assistance of any suitable actuator assembly (e.g., assembly 1118) and/or by sending any suitable data 99 for adjusting any suitable functionality and/or output of an auxiliary condition subsystem 1200 to improve the system's user experience or the user's activity performance capability (e.g., generally and/or with respect to the biomechanical achievement of the user (e.g., to provide additional support when a user is attempting a sit to stand action that is predicted to not be fully successful without such additional support))) and/or for controlling any suitable functionality of any suitable sensor assembly of subsystem 1100 or otherwise (e.g., for turning on or off a particular type of sensor and/or for adjusting the functionality (e.g., the accuracy) of a particular type of sensor (e.g., to gather any additional suitable sensor data)), and/or for updating or supplementing any input data available to activity application 1103a that may be used to determine a planned activity, and/or the like. For example, a particular rule may be a minimum threshold biomechanical achievement (e.g., minimum balance or strength) below which the predicted biomechanical achievement ought to result in a warning or other suitable instruction being provided to the experiencing entity with respect to the unsuitability of the condition of interest with respect to the experiencing entity's biomechanical achievement (e.g., an instruction to stop a particular activity of the condition of interest or not go through with a planned surgery of the condition of interest). A threshold or rule may be determined in any suitable manner and may vary between different experiencing entities and/or between different conditions of interest and/or between different combinations of such experiencing entities and conditions and/or in any other suitable manner

It is to be understood that a user (e.g., experiencing entity) does not have to be physically experiencing (e.g., with user subsystem 1100) a particular condition of interest in order for the biomechanical model to provide a predicted biomechanical achievement (e.g., biomechanical achievement state data) applicable to that condition for that user. Instead, for example, the user may select a particular condition of interest from a list of possible conditions of interest (e.g., conditions previously experienced by the user or otherwise accessible by the model custodian) as well as any suitable time (e.g., time period in the future or the current moment in time)and/or with respect to any suitable planned event for the condition of interest (e.g., after arthroscopic knee surgery by Dr. Doe), and the model custodian may be configured to access any suitable condition category data for that condition of interest (e.g., using any suitable auxiliary condition subsystem data 91 from any suitable auxiliary condition subsystem 1200 determined to be associated with or similar to the condition of interest) in order to determine an appropriate predicted biomechanical achievement for that condition of interest and/or to generate any suitable control data for that predicted biomechanical achievement, which may help the user determine whether or not to experience that condition (e.g., perform a particular activity and/or go through with a particular event).

If a condition of interest is experienced by the experiencing entity, then any suitable condition data (e.g., any suitable user behavior information), which may include an experiencing entity provided biomechanical achievement data, may be detected during that experience and may be stored (e.g., along with any suitable condition characteristic information of that experience) as condition data 1105b and/or may be used in an additional receipt and train loop for further training the learning engine. Moreover, in some embodiments, a biomechanical model custodian may be operative to compare a predicted biomechanical achievement for a particular condition of interest with an actual experiencing entity provided biomechanical achievement for the particular condition of interest that may be received after or while the experiencing entity may be actually experiencing the condition of interest and enabled to actually score or define the biomechanical achievement of the experienced condition of interest (e.g., using any suitable user behavior information, which may define any suitable actual user generated biomechanical achievement data). Such a comparison may be used in any suitable manner to further train the learning engine and/or to specifically update certain features (e.g., weights) of the learning engine. For example, any algorithm or portion thereof that may be utilized to determine a predicted biomechanical achievement may be adjusted based on the comparison. A user (e.g., experiencing entity (e.g., an end user of subsystem 1100)) may be enabled by the biomechanical model custodian to adjust one or more filters, such as a profile of conditions they prefer and/or any other suitable preferences or user profile characteristics (e.g., age, weight, blood pressure, etc.) in order to achieve such results. This capability may be useful based on changes in an experiencing entity's capabilities and/or objectives as well as the biomechanical achievement results. For example, if a user loses its hearing or ability to see, this information may be provided to the model custodian, whereby one or more weights of the model may be adjusted such that the model may provide appropriate predicted biomechanical achievements in the future.

Therefore, any suitable biomechanical model custodian (e.g., subsystem 1100 and/or auxiliary biomechanical subsystem 1250) may be operative to generate and/or manage any suitable biomechanical model or biomechanical learning engine that may utilize any suitable machine learning, such as one or more artificial neural networks, to analyze certain condition data of a condition to predict/estimate the biomechanical achievement of that condition for a particular user of the condition (e.g., generally, and/or at a particular time, and/or with respect to one or more planned activities), which may enable intelligent suggestions be provided to the user and/or intelligent system functionality adjustments be made for improving the user's experience with system 1. For example, a biomechanical engine may be initially configured or otherwise developed for an experiencing entity based on information provided to a model custodian by the experiencing entity that may be indicative of the experiencing entity's specific preferences for different conditions and/or condition types (e.g., generally and/or for particular times and/or for particular planned activities) and/or of the experiencing entity's specific experience with one or more specific conditions. An initial version of the biomechanical engine for the experiencing entity may be generated by the model custodian based on certain assumptions made by the model custodian, perhaps in combination with some limited experiencing entity-specific information that may be acquired by the model custodian from the experiencing entity prior to using the biomechanical engine, such as the experiencing entity's age, weight, height, fastest run mile, health history, and/or the like. The initial configuration of the biomechanical engine may be based on data for several condition categories, each of which may include one or more specific condition category data values, each of which may have any suitable initial weight associated therewith, based on the information available to the model custodian at the time of initial configuration of the engine (e.g., at operation 1302 of process 1300 of FIG. 13). As an example, a condition category may be user age, and the various specific condition category data values for that condition category may include <10 years old, 10-19 years old, 20-39 years old, 40-59 years old, 60-79 years old, 80-99 years old, and ≥100 years old, each of which may have a particular initial weight associated with it.

Once an initial biomechanical engine has been created for an experiencing entity, the model custodian may provide a survey or presentation of requests to the experiencing entity that asks for specific information and/or action performance with respect to a particular condition that the experiencing entity has experienced in the past or which the experiencing entity is currently experiencing. Not only may a survey ask for objective information about a particular condition, such as an identification of the condition, the time at which the condition was/is to be experienced, the current sleep level of the experiencing entity, the current nutrition level of the experiencing entity, the current mindfulness level of the experiencing entity, any suitable health history or vital statistics, an activity type performed by the experiencing entity in the condition, and/or the like, but also for objective information about a user's performance of an activity of the condition (e.g., sensor data indicative of performance of a user activity, such as running or walking or standing up or lifting generally or prior to a planned event (e.g., a surgery), etc.) and/or objective information about any the experiencing entity's actual biomechanical achievement actual length of distance run, sensed walking data after a surgery, etc.) for the condition and/or subjective information from the user about the activity or the condition generally or with respect to different condition characteristics (e.g., the experiencing entity's pain level or difficulty with respect to any portion(s) of the condition) and/or the like. A completed survey may include responses and sensed activity data and actual biomechanical achievement data. Each completed experiencing entity survey for one or more conditions (e.g., one or more conditions generally and/or for one or more times and/or for one or more planned activities) by one or more particular experiencing entity respondents of the experiencing entity may then be received by the model custodian and used to train the biomechanical engine. By training the biomechanical engine with such experiencing entity feedback on one or more prior and/or current condition experiences, the biomechanical engine may be more customized to the experiencing entity by adjusting the weights of one or more condition category options to an updated set of weights for providing an updated biomechanical engine.

Such an updated biomechanical engine, as trained based on experiencing entity survey responses or otherwise, may then be used by the model custodian to identify one or more conditions that may provide a particular experience to an experiencing entity. For example, condition data from each one of one or more available conditions accessible to the system (e.g., to the model custodian), for example, in any suitable condition database that may be accessible in any suitable manner (e.g., by the biomechanical model) may be run through the updated biomechanical engine for the experiencing entity so as to generate a predicted biomechanical achievement for each available condition (e.g., predicted biomechanical achievement data (e.g., distance run, gait properties after a surgery event of a particular type, and/or the like that the engine predicts the experiencing entity would achieve if the experiencing entity were to experience the available condition). If a predicted biomechanical achievement is generated by an experiencing entity's biomechanical engine for a particular available condition that meets a particular threshold (e.g., a user's gait would achieve an appropriate baseline within an appropriate amount of time after a particular surgery event) (e.g., generally or for particular time and/or for a particular planned activity (e.g., surgery) that may be determined to be of possible interest to the experiencing entity, for example, with respect to a condition that may be possibly experienced by the experiencing entity now or in the future), then the model custodian may utilize that information in any suitable way to facilitate suggesting or otherwise leading the experiencing entity to the particular available condition. Therefore, a model custodian may be used to determine a biomechanical achievement match between a user and a particular available condition and to facilitate utilization of a such a determined match. If a user and a condition are matched, any suitable feedback (e.g., condition data (e.g., condition characteristic information, user behavior information, user condition preference(s), and/or the like)) may be obtained by the model custodian (e.g., while the user prepares to experience the condition, during the user's experience of the condition, and/or after the user's experience of the condition) to bolster any suitable condition data associated with that experience in any suitable experience database that may be associated with the model (e.g., in any suitable condition database) and/or to further train the biomechanical model. Therefore, the biomechanical engine may be continuously refined and updated by taking into account all feedback provided by any experiencing entity, such that the experiencing entity's biomechanical engine may be improved for generating more accurate predicted biomechanical achievements going forward for future potential condition experiences. A model custodian may manage not only a condition database and one or more various biomechanical models (e.g., for one or more different experiencing entities), but also any and/or all connections and/or experiences between experiencing entities and conditions, such that the model custodian may be a master interface for all the needs of any experiencing entity and/or of any condition custodian (e.g., a physician of a hospital or a physical therapist for a specific location or the like that may benefit from any data that such a model custodian may be able to provide such a condition custodian (e.g., to improve the quality and/or popularity of the condition (e.g., to recommend or not recommend certain surgeries to certain users)).

It is to be understood that subsystem 1100 may be a model custodian for at least a portion or all of model 1105a and/or for at least a portion or all of model 1255a at the same time and/or at different times, and/or subsystem 1250 may be a model custodian for at least a portion or all of model 1105a and/or for at least a portion or all of model 1255a at the same time and/or at different times. Model 1105a may be for one or more particular users (e.g., one or more particular users associated with (e.g., registered to) subsystem 1100) while model 1255a may be for a larger group of experiencing entities, including those of model 1105a as well as other users (e.g., users of various other user subsystems that may be within system 1 (not shown (e.g., within a user subsystem ecosystem)). At least a portion of model 1255a may be used with at least a portion of model 1105a (e.g., as a hybrid model) in any suitable combination for any suitable purpose, or model 1255a may be periodically updated with any suitable model data from model 1105a or vice versa. Alternatively, model 1105a and model 1255a may be identical and only one may be used (e.g., by subsystem 1100) for a particular use case.

To accurately predict the biomechanical achievement that may be provided by a user for a condition, any suitable portion of system 1, such as subsystem 1100, may be configured to use various information sources in combination with any available biomechanical model in order to characterize or classify or predict a biomechanical achievement of a user of subsystem 1100 when appropriate or when possible. For example, any suitable processing circuitry or assembly (e.g., a biomechanical module) of subsystem 1100 may be configured to gather and to process various types of condition data, in conjunction with a biomechanical model, to determine what type of biomechanical achievement is to be expected for a particular condition. For example, any suitable condition data from one or more of sensor assembly 1114 of subsystem 1100, auxiliary condition subsystem 1200 (e.g., from one or more assemblies thereof), activity application 1103a of subsystem 1100, and/or condition data 1105b of subsystem 1100 may be utilized in conjunction with any suitable biomechanical model, such as with device biomechanical model 1105a and/or auxiliary biomechanical model 1255a of auxiliary biomechanical subsystem 1250 to determine a biomechanical achievement state of a user efficiently and/or effectively.

FIG. 12 shows a schematic view of a biomechanical management system 1201 (e.g., of user subsystem 1100) of system 1 that may be provided to manage biomechanical achievements of a user of subsystem 1100 (e.g., to determine a biomechanical achievement of a user of subsystem 1100 and to manage a mode of operation of subsystem 1100 and/or of any other suitable subsystem of system 1 based on the determined biomechanical achievement). In addition to or as an alternative to using device sensor assembly data 1114′ that may be generated by device sensor assembly 1114 based on any sensed condition characteristics, biomechanical management system 1201 may use various other types of data accessible to subsystem 1100 in order to determine a current biomechanical achievement of a user of subsystem 1100 in a particular condition and/or to determine a predicted biomechanical achievement of a user in an available condition in conjunction with any suitable biomechanical model (e.g., in conjunction with model 1105a and/or model 1255a), such as any suitable data provided by one or more of auxiliary condition subsystem 1200 (e.g., data 91 from one or more assemblies of auxiliary condition subsystem 1200), activity application 1103a of subsystem 1100 (e.g., data 1103a′ that may be provided by application 1103a and that may be indicative of one or more planned activities), and/or condition data 1105b (e.g., any suitable condition data 1105b′ that may be any suitable portion or the entirety of condition data 1105b). In response to determining the current biomechanical achievement for a current condition or a predicted biomechanical achievement for a potential available condition, biomechanical management system 1201 may apply at least one biomechanical achievement-based mode of operation to at least one managed element 1290 (e.g., any suitable assembly of subsystem 1100 and/or any suitable assembly of subsystem 1200 and/or any suitable assembly of subsystem 1250 or otherwise of system 1) based on the determined biomechanical achievement (e.g., to suggest certain user behavior and/or to control the functionality of one or more system assemblies) for improving a user's experience with system 1. For example, as shown in FIG. 12, biomechanical management system 1201 may include a biomechanical module 1240 and a management module 1280.

Biomechanical module 1240 of biomechanical management system 1201 may be configured to use various types of accessible data in order to determine (e.g., characterize) a biomechanical achievement or biomechanical achievement state (e.g., a current biomechanical achievement or current biomechanical achievement state of a user of subsystem 1100 within a current condition and/or a potential biomechanical achievement state of a user within a potential available condition). As shown, biomechanical module 1240 may be configured to receive any suitable device sensor assembly data 1114′ that may be generated and shared by any suitable device sensor assembly 1114 based on any sensed condition characteristics (e.g., automatically or in response to any suitable request type of device sensor request data 1114″ that may be provided to sensor assembly 1114 (e.g., by module 1240)), any suitable auxiliary condition subsystem data 91 that may be generated and shared by any suitable auxiliary condition subsystem assembly(ies) based on any sensed condition characteristics or any suitable auxiliary subsystem assembly characteristics (e.g., automatically or in response to any suitable request type of auxiliary condition subsystem data 99′ that may be provided to auxiliary condition subsystem 1200 (e.g., by module 1240)), any suitable activity application status data 1103a′ that may be generated and shared by any suitable activity application 1103a that may be indicative of one or more planned activities (e.g., automatically or in response to any suitable request type of activity application request data 1103a″ that may be provided to activity application 1103a (e.g., by module 1240)), and/or any suitable condition data 1105b′ that may be any suitable shared portion or the entirety of condition data 1105b (e.g., automatically or in response to any suitable request type of condition request data 1105b″ that may be provided (e.g., by module 1240) to a provider of condition data 1105b (e.g., memory assembly 1104)), and biomechanical module 1240 may be operative to use such received data in any suitable manner in conjunction with any suitable biomechanical model to determine any suitable biomechanical achievement state (e.g., with device biomechanical model data 1105a′ that may be any suitable portion or the entirety of device biomechanical model 1105a, which may be accessed automatically and/or in response to any suitable request type of device biomechanical model request data 1105a″ (e.g., condition data and actual achievement data for training or condition data for requesting prediction) that may be provided (e.g., by module 1240) to a provider of device biomechanical model 1105a (e.g., memory assembly 1104), and/or with auxiliary biomechanical subsystem model data 81 that may be any suitable portion or the entirety of auxiliary biomechanical model 1255a, which may be accessed automatically and/or in response to any suitable request type of auxiliary biomechanical subsystem request data 89′ that may be provided (e.g., by module 1240) to a provider of auxiliary biomechanical model 1255a (e.g., auxiliary biomechanical subsystem 1250)).

Once biomechanical module 1240 has determined a current biomechanical achievement for a current condition or a predicted biomechanical achievement for a potential available condition (e.g., based on any suitable combination of one or more of any suitable received data 1114′, 91, 1103a′, 1105b′, 1105a′, and 81), biomechanical module 1240 may be configured to generate and transmit biomechanical achievement state data 1222 to management module 1280, where biomechanical achievement state data 1222 may be indicative of the determined biomechanical achievement for the user of subsystem 1100. In response to determining a biomechanical achievement of a user of subsystem 1100 by receiving biomechanical achievement state data 1222, management module 1280 may be configured to apply at least one biomechanical achievement-based mode of operation to at least one managed element 1290 of system 1 (e.g., of subsystem 1100) based on the determined biomechanical achievement. For example, as shown in FIG. 12, biomechanical management system 1201 may include management module 1280, which may be configured to receive biomechanical achievement state data 1222 from biomechanical module 1240, as well as to generate and share biomechanical achievement mode data 1224 with at least one managed element 1290 of subsystem 1100 and/or of any other suitable subsystem of system 1 at least partially based on the received biomechanical achievement state data 1222 and any suitable rule system or management control application (e.g., application 1103, 1103a, etc.) that may be operative to process data 1222 for generating appropriate data 1224 to appropriately control element 1290, where such biomechanical achievement mode data 1224 may be received by managed element 1290 and used for controlling at least one characteristic of managed element 1290. Managed element 1290 may be any suitable assembly of subsystem 1100 (e.g., any processor assembly 1102, any memory assembly 1104 and/or any data stored thereon, any communications assembly 1106, any power supply assembly 1108, any input assembly 1110, any output assembly 1112, any sensor assembly 1114, any actuator assembly 1118, etc.) and/or any suitable assembly of any suitable auxiliary condition subsystem 1200 of system 1 and/or any suitable assembly of any suitable auxiliary biomechanical subsystem 1250 of system 1 and/or of any other suitable subsystem (e.g., another user subsystem (e.g., a physician's personal computing device) of system 1 and/or the like), and biomechanical achievement mode data 1224 may control managed element 1290 in any suitable way, such as by enhancing, enabling, disabling, restricting, and/or limiting one or more certain functionalities associated with such a managed element.

Biomechanical achievement mode data 1224 may be any suitable device control data for controlling any suitable functionality of any suitable assembly of subsystem 1100 as a managed element 1290 (e.g., any suitable device output control data for controlling any suitable functionality of any suitable output assembly 1112 of subsystem 1100 (e.g., for adjusting a user interface presentation to user U (e.g., to provide a biomechanical achievement suggestion)) and/or any suitable device sensor control data (e.g., a control type of device sensor request data 1114″) for controlling any suitable functionality of any suitable sensor assembly 1114 of subsystem 1100 (e.g., for turning on or off a particular type of sensor and/or for adjusting the functionality (e.g., the accuracy) of a particular type of sensor (e.g., to gather any additional suitable sensor data)) and/or any suitable device actuator control data for controlling any suitable functionality of any suitable actuator assembly 1118 of subsystem 1100 (e.g., for adjusting any support and/or assistance that may be provided by actuator assembly 1118 to the user of subsystem 1100 (e.g., to provide biomechanical support when a predicted biomechanical achievement is determined to be inadequate for achieving a desired result (e.g., transitioning from sit to stand))) and/or any suitable activity application control data (e.g., a control type of activity application request data 1103a″) for updating or supplementing any input data available to activity application 1103a that may be used to determine a planned activity, and/or the like). Additionally or alternatively, biomechanical achievement mode data 1224 may be any suitable auxiliary condition subsystem data 99 for controlling any suitable functionality of any suitable auxiliary condition subsystem 1200 as a managed element 1290 in an environment of the user (e.g., exercise equipment, sports equipment, and/or the like). Additionally or alternatively, biomechanical achievement mode data 1224 may be any suitable auxiliary biomechanical subsystem data 89 for providing any suitable data to auxiliary biomechanical subsystem 1250 as a managed element 290 (e.g., any suitable auxiliary biomechanical subsystem data 89 for updating auxiliary biomechanical model 1255a of auxiliary biomechanical subsystem 1250 in any suitable manner). Additionally or alternatively, biomechanical achievement mode data 1224 may be any suitable device biomechanical model update data (e.g., an update type of device biomechanical model request data 1105a″) for providing any suitable data to device biomechanical model 1105a as a managed element 1290 (e.g., any suitable device biomechanical model update data 1105a″ for updating device biomechanical model 1105a in any suitable manner). Additionally or alternatively, biomechanical achievement mode data 1224 may be any suitable device condition update data (e.g., an update type of condition request data 1105b″) for providing any suitable update data to condition data 1105b as a managed element 1290 (e.g., any suitable condition update data 1105b″ for updating condition data 1105b in any suitable manner).

FIG. 13 is a flowchart of an illustrative process 1300 for managing a biomechanical achievement. At operation 1302 of process 1300, a biomechanical model custodian system may initially configure a learning engine (e.g., system 1 may configure device biomechanical model 1105a or auxiliary comfort model 1255a (e.g., generally or for a particular experiencing entity)). At operation 1304 of process 1300, the biomechanical model custodian system may receive condition category data for at least one condition category for a first condition of a first experiencing entity and achievement data for an actual achievement of the first experiencing entity for the first condition. At operation 1306 of process 1300, the biomechanical model custodian system may train the learning engine using the received condition category data (e.g., as input(s)) and the received achievement data (e.g., as output(s) or label(s) of the input data). At operation 1308 of process 1300, the biomechanical model custodian system may access condition category data for the at least one condition category for a second condition of a second experiencing entity (e.g., an entity that may be the first experiencing entity or an experiencing entity different than the first experiencing entity). At operation 1310 of process 1300 (e.g., after the learning engine has been trained at least at operation 1306), the biomechanical model custodian system, using the learning engine, may predict an achievement of the second experiencing entity with the accessed condition category data for the second condition. At operation 1312 of process 1300, when the predicted achievement for the second condition satisfies a rule, the biomechanical model custodian system may generate control data associated with the satisfied rule. At operation 1314 of process 1300, the biomechanical model custodian system may control a functionality of a managed element of the biomechanical model custodian system using the generated control data.

It is understood that the operations shown in process 1300 of FIG. 13 are only illustrative and that existing operations may be modified or omitted, additional operations may be added, and the order of certain operations may be altered.

Various types of wearable sensor technologies (e.g., as described above with respect to FIGS. 1A-13) are opening up new opportunities and applications across multiple areas, including digital health, fitness, and industrial operations. These sensor technologies are generating large volumes of new types of data, spurring a new revolution in data science and services. However, while data is being generated at an unprecedented rate, certain sensor technologies may not offer high enough resolution for measuring biomechanical variables that can be used to make meaningful determinations, including, but not limited to, making determinations in health outcomes, disease severity state, understanding important user mobility gait biomechanics (e.g., when walking or running), and/or the like. For example, the health and wellness of a user can be correlated with various gait biomechanical markers, including, but not limited to, step length, stride length, stride speed, gait speed, cadence, cadence variability, and ground contact time (e.g., any suitable movements 630 of FIG. 6). Such biomechanical markers can be used to make various determinations about a user, including, but not limited to, the fall risk of a user, an assessment about the post-recovery of a surgery, a characterization of the behavior of a movement disorder, and/or whether a patient has an asymmetric stride between the left leg and right leg, a shuffle gait, or is gradually walking more consistently and faster. Therefore, solutions to measure real-world biomechanical gait markers are provided herein that can be used in various ways, such as to characterize the health and wellness of an individual, measure the outcome of patient surgeries that impact mobility (e.g., arthroplasty), determine if an individual is on a proper recovery path, and/or intervene when a patient is not on a proper recovery path.

Additionally or alternatively, in the sport of running, base metrics that may be of interest may include, but are not limited to, an individual include pace, time, and distance. While GPS or other location and time-based detection systems may accurately compute pace and distance traveled by a user, such systems must be carried by the user and are often bulky and require significant battery resources. Therefore, solutions to measure real-world step length and gait speed are provided herein that can be used without the need for GPS data or other such data, such as by using a sensor and technology platform that may generate and process various biomechanical signals, including, but not limited to cadence, vertical displacement of the pelvis, horizontal velocity changes of the pelvis, pelvic transverse rotation, sagittal tilt, and/or coronal drop (e.g., any suitable movements 630 of FIG. 6), in order to estimate with high accuracy, for example, the step length and gait speed of a user (e.g., when walking or running).

As described above with respect to FIGS. 1A-13, an activity monitoring platform or system may include hardware, software algorithms, applications, and/or web services that can automatically calibrate wearable or otherwise user-transportable sensors and detect the location of the sensor on the user. Additionally or alternatively, such an activity monitoring platform or system may include a secure cloud database that may store some or all user data that can be synced or shared in any suitable and appropriate manner to better identify where future sensor devices may be located or help tune location identification models. Such hardware may include a device that can be worn on a wearer's body, embedded into garments, belts, and/or other equipment worn on the body. Depending on the specific application, a sensor can be worn on the waist, pelvis, upper body, shoes, thigh, arms, wrists, or head. If worn on the wrist or arm of a user, the device can be embedded into a watch, wrist band, elbow sleeve, or arm band. An additional device may be used and clipped on the other wrist or arm, or placed on the waist on the pelvis, or slipped into a pocket in the garment, embedded into the garment itself, back-brace, belt, hat, glasses, or other products the user may be wearing. A device can also be an adhesive patch worn on the skin. Other form factors can also clip onto the shoe or be embedded into a pair of socks or a shoe itself. The system may include one sensor or multiple sensors that may be communicatively or otherwise coupled together. The system may include an accelerometer, gyroscope, magnetometer, altimeter sensor, and/or any other suitable sensor assemblies, and/or a Bluetooth chip with RF antenna and/or any other suitable communication assemblies. Some instances may also contain GPS, electromyography (EMG), electrocardiography (ECG), and capacitive touch sensors, while other instances may only contain a single triaxial accelerometer with CPU, Bluetooth chip and RF antenna. The processing can take place on the device itself or be wirelessly transmitted to a smartphone, smartwatch, computer, or web server that may process the biomechanical signals and forces on the human body. The device can also communicate over Bluetooth, and/or over 2G/3G/4G/5G/LTE and/or any other suitable telecommunications network(s). The device may include a haptic vibration motor, bright LED lights, and/or audio speaker for real-time feedback. The system may include software applications that can run on a computer, smartphone, or cloud server, and that may allow a user to sync data from the device, configure the device and settings, and/or view the data from the device. The software applications can also process the raw signals from the device and communicate with a webserver that may sync data and/or send firmware updates.

When a user wears any suitable sensor, the sensor may be configured to detect user motion and wake up from a sleep mode. When the user begins walking or otherwise moving, the sensor and system may be configured to generate a reference orientation frame before it may begin location detection. A method for auto-calibration has been described in U.S. Patent Application Publication No. 2017-0258374, titled “SYSTEM AND METHOD FOR AUTOMATIC POSTURE CALIBRATION,” published on Sep. 14, 2017, which is hereby incorporated by reference herein in its entirety. In addition to auto-calibration, one, some, or each sensor that may be carried by a user may synchronize to the same time. This can be accomplished by connecting with each other and/or with a peripheral device, such as a smart phone or smart watch that may include a reliable real-time clock.

Various systems and methods may be used for calculating various gait (e.g., walking gait, running, gait, etc.) biomechanical signals and/or gait biomechanical markers (e.g., cadence, vertical displacement of the pelvis, horizontal velocity of the pelvis, pelvic transverse rotation, pelvic tilt, pelvic drop, etc.) including, but not limited to, those described herein and in one or more of U.S. Patent Application Publication No. 2017-0095181, titled “SYSTEM AND METHOD FOR CHARACTERIZING BIOMECHANICAL ACTIVITY,” published on Apr. 6, 2017, U.S. Patent Application Publication No. 2017-0095692, titled “SYSTEM AND METHOD FOR RUN TRACKING WITH A WEARABLE ACTIVITY MONITOR,” published on Apr. 6, 2017, and U.S. Patent Application Publication No. 2017-0273601, titled “SYSTEM AND METHOD FOR APPLYING BIOMECHANICAL CHARACTERIZATIONS TO PATIENT CARE,” published on Sep. 28, 2017, each of which is hereby incorporated by reference herein in its entirety. For example, various biomechanical gait markers can be computed from a sensor placed on a single location with respect to the user's body or from multiple sensors placed at multiple locations with respect to the user's body. Sensor location(s) may depend on the injury type or specific biomechanical marker(s) to be measured. For example, a device can be placed on the pelvis to measure pelvic rotation dynamics, as well as vertical, horizontal, and lateral displacements of the pelvis, and/or the like. In another example, a sensor on a foot can be used to determine the vertical lift of the foot during a step, stride length, stride speed, foot pronation, impact force, ground contact time, and/or the like. For example, a sensor on the pelvis can be used to measure a hip arthroplasty and a sensor on the foot can be used to measure an ankle arthroplasty on the involved leg. Multiple sensors can also be worn or otherwise carried across a user's body, such as one sensor on the pelvis, and another sensor on the foot. These sensors can be connected to provide a more comprehensive biomechanical picture of the patient. For instance, both sensors can be synchronized to measure the biomechanical signals and forces associated with each individual foot step. The forces can be compared and computed to infer additional biomechanical signals. For example, a vertical displacement computed from a foot sensor and a vertical displacement computed from a pelvis sensor can be used to infer an estimate of the overall knee flexion of the left or right leg. Knee flexion can be used as a biomechanical gait marker to predict the recovery rate of a patient that had knee arthroplasty. In another example, two sensors can be worn, one on the left foot, one on the right foot. These sensors can be used to calculate the asymmetries in stance times between the left and right leg. Additionally, both sensors can synchronize stance times to compute double-stance times. Double-stance time is an impact biomechanical signal that may be correlated with Parkinson's Disease severity and falling risk. In another example, three sensors can be worn, such as with one on the left foot, one on the right foot, and one on the pelvis. Synchronizing these three sensors together with a reference clock, either by connecting with each other or through a smartphone (or other) computing device, can compute asymmetries in single stance times between left and right leg, knee flexion angles between left and right leg, and double-stance times. In addition, sensors can detect a suite of stride-based biomechanical signals, including, but not limited to, step cadence (e.g., number of steps per minute), ground contact time, left and/or right foot stance time, double-stance time, forward/backward braking forces, upper body trunk lean, upper body posture, step duration, step length, swing time, step impact or shock, activity transition time, stride symmetry/asymmetry, stride speed, left or right foot detection, pelvic dynamics (e.g., pelvic stability; range of motion in degrees of pelvic drop, tilt and rotation, vertical displacement/oscillation of the pelvis, and/or lateral displacement/oscillation of the pelvis), motion path, balance, turning velocity and peak velocity, foot pronation, vertical displacement of the foot, neck orientation, tremor quantification, shuffle detection, and/or any other suitable gait or biomechanical metrics. Any suitable sensor assembly at least partially worn on a user (e.g., pelvis and/or otherwise) and associated system or platform sensor may be configured to compute these and several other (e.g., walking and miming) biomechanical gait signals with every user step (e.g., along with any suitable demographic information). Biomechanically, various gait metrics, such as at least some of the ones mentioned herein, can be closely tied to step size.

Various biomechanical algorithms, models, and/or the like may be used by any suitable biomechanical achievement system with any suitable sensor assembly(ies) worn or otherwise carried by a user to determine various biomechanical movement metrics or markers of the user for any suitable purpose (e.g., to control the functionality of any suitable managed element 1290). For example, when a sensor is worn on the pelvis, foot, and/or any other place on the user's body, a number of biomechanical signals can be generated and used to set a biomechanical gait baseline across various demographic groups to determine normal gait characteristics and abnormal gait characteristics and/or pre-surgery gait characteristics and post-surgery gait characteristic. While this may be done in an artificial environment, such as a gait-lab that may require a lot of time and resources that can constrain the population baseline size, wearable sensor assemblies (e.g., any suit described herein or other sensor assembly that may easily be worn or otherwise carried by a user in its day to day activities, may enable scaling the population to much larger numbers and capture data over longer periods of time, especially for enabling capturing data in the real-world and under normal walking or running or other moving conditions, as opposed to a limited amount of data captured in an artificial environment where the users may be walking in their “best behavior” that does not actually reflect their normal behavior. Patients can wear a sensor or multiple sensors to monitor and establish their own personal baseline over a few days to weeks before a surgical operation. This may establish a pre-surgery baseline that can be compared to data captured post-surgery. After the surgery, the patient can wear the same sensors daily to measure their recovery progress until they are fully recovered. Often, certain post-surgery biomechanical gait signals may get worse relative to a pre-surgery baseline for a few days to weeks while a patient is recovering, limping, using a crutch, and/or the like. But over time, the patient usually improves, and the biomechanical gait markers should improve and gradually establish a new or similar baseline.

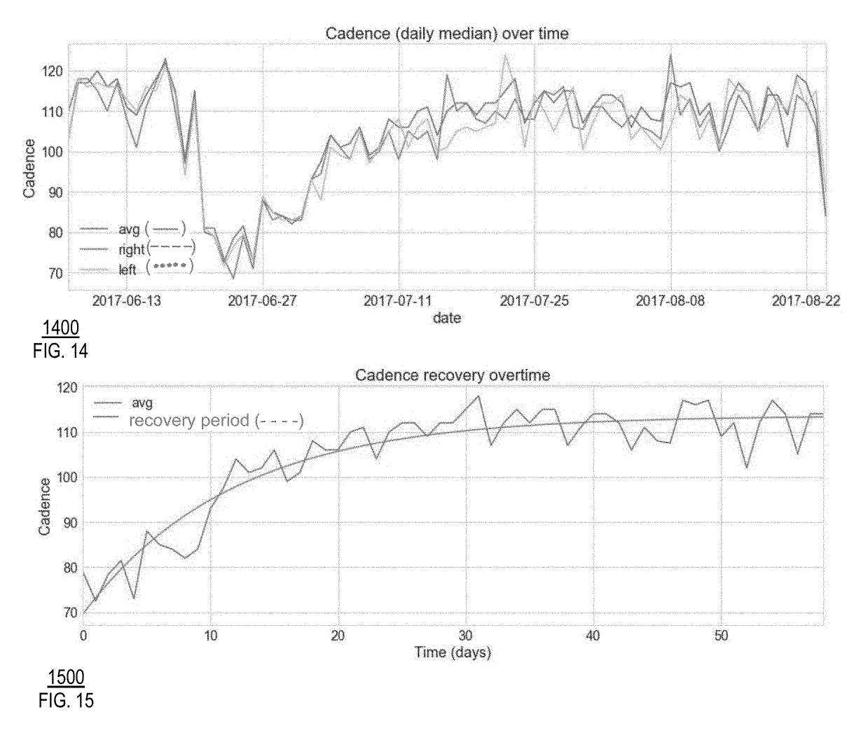

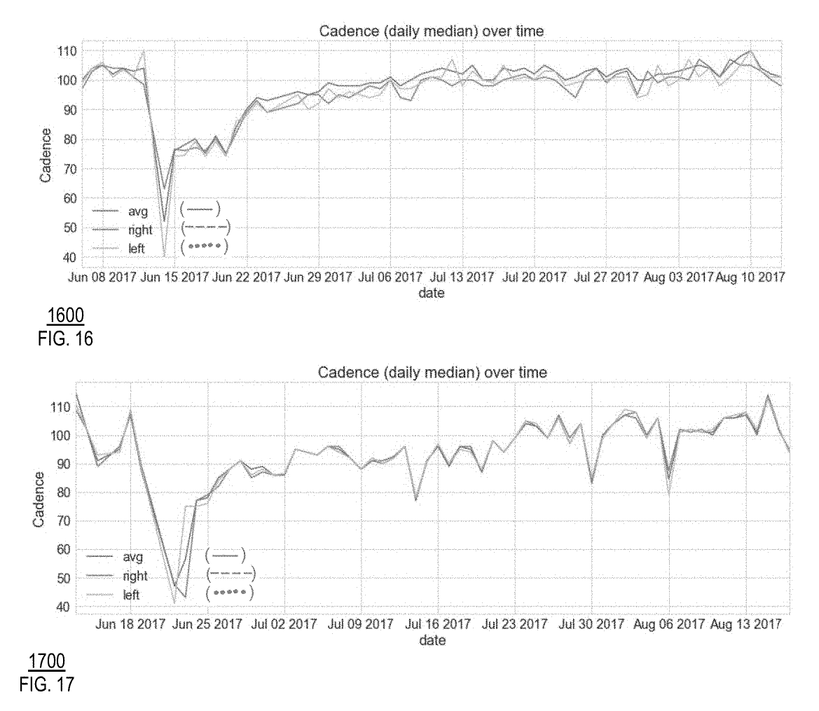

As shown by graph 1400 of FIG. 14, various biomechanical gait markers may be measured by a sensor assembly worn by a user to measure the cadence or any other suitable biomechanical movement(s) of the user over time (e.g., before and after a surgery (or any other suitable event) on Jun. 20, 2017). Cadence may be the number of steps taken per minute and can be estimated on a per step basis, quantified over an entire minute, or averaged over a certain period of time. As shown, separate cadence values may be computed for the left foot (e.g., green or dotted line), right foot (e.g., red or dashed line), and averaged over a stride, such as one left foot step and one right foot step (e.g., blue or solid line), where a cadence baseline before the surgery was around 110-115 steps per minute, and where this cadence marker falls dramatically to around 70 steps per minute immediately after the surgery, and gradually increases back to a cadence baseline of around 105-110 steps per minute. As shown by graph 1500 of FIG. 15, the same averaged stride patient cadence data from graph 1400 following the surgery may be provided but also with a dashed line that has been fitted to model the recovery period. In this case, the recovery time took about 30 days to establish a new biomechanical cadence baseline. Other patients may also exhibit a similar behavior in the drop and subsequent gradual recovery of the cadence biomechanical marker after a particular type of surgery or other event similar to that of the patient of FIGS. 14 and 15. For example, as shown by graph 1600 of FIG. 16 for another patient, with a surgery on Jun. 13, 2017, separate cadence values may be computed for the left foot (e.g., green or dotted line), right foot (e.g., red or dashed line), and averaged over a stride, such as one left foot step and one right foot step (e.g., blue or solid line), where a cadence baseline before the surgery was around 105 steps per minute, and where this cadence marker falls dramatically to around 55 steps per minute immediately after the surgery, and gradually increases back to a cadence baseline of around 100 steps per minute, where the patient recovered to about 90% of his new baseline in about two weeks after the surgery. As another example, as shown by graph 1700 of FIG. 17 for yet another patient, with a surgery on Jun. 19, 2017, separate cadence values may be computed for the left foot (e.g., green or dotted line), right foot (e.g., red or dashed line), and averaged over a stride, such as one left foot step and one right foot step (e.g., blue or solid line), where a cadence baseline before the surgery was around 105 steps per minute, and where this cadence marker falls dramatically to around 50 steps per minute immediately after the surgery, and gradually increases back to a cadence baseline of around 100 steps per minute, where the patient recovered to about 90% of his new baseline in about one month after the surgery.

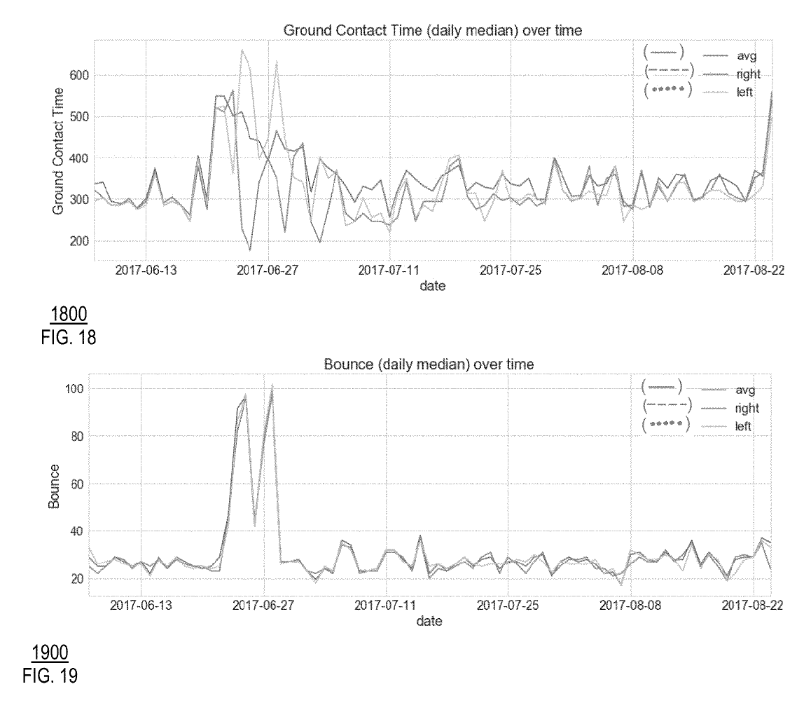

In addition to or as an alternative to a cadence biomarker, various other markers can exhibit a meaningful change before and after a particular type of surgery or event. Some other metrics may include, but are not limited to, ground contact time, left leg and right leg asymmetry, vertical oscillation of the pelvis, stride length, stride speed, and/or the like (e.g., any biomechanical movement or combination of biomechanical movements 630 of FIG. 6). For example, as shown by graph 1800 of FIG. 18 for a patient, with a surgery or event on Jun. 21, 2017, separate ground contact time values (e.g., in milliseconds) may be computed for the left foot (e.g., green or dotted line), right foot (e.g., red or dashed line), and averaged over a stride, such as one left foot step and one right foot step (e.g., blue or solid line) to indicate stride asymmetry, the average ground contact time values may increase significantly from an average of 300 milliseconds prior to the surgery to an average of 500 milliseconds after the surgery. Specifically, the left leg may increase to above 600 milliseconds while the right leg may decrease to below 200 milliseconds, which may indicate an asymmetry between the involved and uninvolved leg, where, in this case, the involved leg is likely to be the right leg because the patient spent significantly less time on this leg. Additionally, this same patient also exhibits a large increase in vertical displacement (e.g., bounce) after the surgery, as may be demonstrated by graph 1900 of FIG. 19, where separate bounce values (e.g., in millimeters) may be computed for the left foot (e.g., green or dotted line), right foot (e.g., red or dashed line), and averaged over a stride, such as one left foot step and one right foot step (e.g., blue or solid line). As shown, after the large increase in vertical displacement from between 20-30 millimeters pre-surgery to between 40-100 millimeters immediately after surgery, the vertical displacement then drops back to a normal baseline of between 20-40 millimeters. From this it may be that the patient began using crutches for a period of time after the surgery (e.g., until Jul. 1, 2017), which can coincide with the sharp discontinuous increase and decrease in vertical displacement beyond normal levels.

Therefore, there may be a number of biomechanical markers that can help provide deeper detail and insight into a patient's recovery after a particular surgery or event. A pre-surgery baseline can be used as a reference baseline to compare with a new (e.g., hopefully healthier) post-surgery baseline. Both of these baselines can also be compared to other relevant population baselines in the future. In the examples of FIGS. 14-19, the patients recovered from the surgery at issue, however, the sensor(s) and system can be used to predict and detect if there is a lapse in patient recovery. For example, module 1240 may receive pre-event and post-event biomechanical marker data for a particular user or person or patient of interest (POI) for a particular type of event of interest (EOI) or POI event (e.g., any suitable surgery or other event to be analyzed for the POI), such as from data 1114′ from any suitable device sensor assembly(ies) 1114 of the POI and/or from any other suitable source(s), as well as pre-event and post-event biomechanical marker data for one or more various different users (DUs) (e.g., user(s) other than the POI) that may be accessible with respect to the same or substantially similar type of event as the EOI from which the DUs properly recovered, such as from data 99 from subsystem 1200 or elsewhere, in order to identify pre-event data from one or more DUs that match or substantially match that of the POI (e.g., any suitable pre-event baseline(s)) and/or to compare the post-event data from the identified DUs to the post-event data of the POI (e.g., any suitable post-event baseline(s)) in order to detect any suitable difference(s) or similarities therebetween as data 1222 that module 1280 may then use to generate any suitable data 1224 for controlling the functionality of any suitable managed element 1290. Therefore, by monitoring the post-surgery recovery period, time to establish a new baseline, and comparison of the new baseline can all be used to detect improper recovery of a POI compared to one or more DUs and to alert a care provider to intervene. If a lapse in recovery was detected, then a healthcare provider can be alerted (e.g., by data 1224) to check in with the patient or the patient can be notified (e.g., by data 1224) to call their care provider. In addition, the system could also automatically suggest (e.g., by data 1224) different times for clinical visits and share them (e.g., by data 1224) with the patient to reduce the time to scheduling an appointment. Therefore, the sensor assembly and system and accessible biomechanical signals from a POI and DUs can be used to analyze or predict recovery times for each individual. One approach may be to compare pre- and post-surgery baselines of one or more biomechanical markers of a POI and at least one DU (or average of multiple DUs) to each other. If a post-surgery baseline fails to improve compared to the pre-surgery baseline, then a care provider can be alerted. Additionally, if the recovery period fails to improve after a steep drop in various biomechanical signals (e.g., as discussed with respect to cadence, ground contact time, vertical displacement, etc. with respect to FIGS. 14-19), then a care provider can be alerted. If at any time it is determined that a recovery of the POI may be stalling, a healthcare provider can be alerted to call the patient and schedule a clinical visit.

Another approach may be to utilize any suitable machine learning techniques to predict a recovery rate or any suitable post-surgery biomechanical data for a POI for any suitable surgery or EOI. With enough high quality patient labeled data, machine learning models can be built using any suitable features, such as the biomechanical signals discussed above or additional features that a model may identify and extract. The labeled data can be used to train a model and predict recovery rates based on demographic information, surgery type, implant type (or implant model, if applicable), and even the doctor that performed the surgery. For example, at least one biomechanical model may be trained (e.g., at operation 1306) not only by condition category data received (e.g., at operation 1304) for at least one or more condition categories for a DU condition of any suitable DU (e.g., as input data for the model) but also by any suitable actual achievement data received (e.g., at operation 1304) for an actual achievement of the DU for the DU condition (e.g., as output for the model), where the DU condition may include the DU experiencing a particular DU event (e.g., a particular surgery or physical therapy or otherwise that may affect the biomechanics of the user). For example, such output data (e.g., actual achievement data) may include any suitable sensor data indicative of any suitable sensed biomechanical movement(s) of the DU for any suitable period of time after a particular DU event, while such input data (e.g., condition category data) may include any suitable sensor data indicative of any suitable sensed biomechanical movement(s) of the DU for any suitable period of time leading up to the particular DU event as well as any other suitable input data associated with the DU and/or with the particular DU event, including, but not limited to, any suitable demographic information about the DU (e.g., age of the DU, occupation of the DU, ethnicity of the DU, where DU lives, etc.), any suitable health information about the DU (e.g., height of the DU, weight of the DU, any diagnosed physical ailments or conditions of the DU, any previous events (e.g., surgeries, and/or the like) experienced by the DU prior to the particular event, measured strength of one, some, or each limb and/or muscle group of the DU, and/or the like), any suitable information about the particular event (e.g., surgery or therapy type, product type (e.g., implant type (if applicable) and/or implant model (if applicable) and/or medication used (if applicable)), name of doctor that performed event on the DU, name of hospital at which the event was performed on the DU, any medications and/or therapy administered during the event and/or after the event, and/or the like), and/or the like. For example, such output data (e.g., actual achievement data) may include any suitable sensor data indicative of any suitable biomechanical movement(s) of a particular DU sensed by any suitable sensor assembly(ies) for any suitable period of time after a particular event (e.g., the cadence values of graph 1400 of the left foot, the right foot, and/or the average stride of a first particular DU after a first particular DU event on Jun. 20, 2017; the cadence values of graph 1600 of the left foot, the right foot, and/or the average stride of a second particular DU after a second particular DU event on Jun. 13, 2017; the cadence values of graph 1700 of the left foot, the right foot, and/or the average stride of a third particular DU after a third particular DU event on Jun. 19, 2017; the ground contact time values of graph 1800 of the left foot, the right foot, and/or the average stride of a fourth particular DU after a fourth particular DU event on Jun. 21, 2017 and/or the bounce values of graph 1900 of the left foot, the right foot, and/or the average stride of the fourth particular DU prior to the fourth particular DU event on Jun. 21, 2017; and/or the like), while such input data (e.g., condition category data) may include any suitable sensor data indicative of any suitable biomechanical movement(s) of the particular DU sensed by any suitable sensor assembly(ies) for any suitable period of time leading up to the particular DU event on event (e.g., the cadence values of graph 1400 of the left foot, the right foot, and/or the average stride of the first particular DU prior to the first particular DU event on Jun. 20, 2017; the cadence values of graph 1600 of the left foot, the right foot, and/or the average stride of the second particular DU prior to the second particular DU event on Jun. 13, 2017; the cadence values of graph 1700 of the left foot, the right foot, and/or the average stride of the third particular DU prior to the third particular DU event on Jun. 19, 2017; the ground contact time values of graph 1800 of the left foot, the right foot, and/or the average stride of the fourth particular DU prior to the fourth particular DU event on Jun. 21, 2017 and/or the bounce values of graph 1900 of the left foot, the right foot, and/or the average stride of the fourth particular DU prior to the fourth particular DU event on Jun. 21, 2017; and/or the like) as well as any other suitable condition category data related to the particular DU and/or to the particular DU event. If the particular DU event of each one of the four particular DU events of FIGS. 14-19 were the same type of event but just on different days (e.g., arthroscopic surgery on the right knee of each particular DU (e.g., by the same doctor at the same hospital), etc.), then a single model associated with that particular type of event may be trained (e.g., at operation 1306) by the input and output data associated with each of the four particular DUs. Additionally or alternatively, the input and output data associated with a particular DU event and a particular DU may train its own model (e.g., at operation 1306). It is to be appreciated that the more sets of input data and output data that become accessible (e.g., at operation 1304), one or more models may become better trained (e.g., at operation 1306) and/or may be trained more specifically to a particular type of category data (e.g., users between the ages of 20 and 30, left knee arthroscopic surgery, or the like) or to a particular combination of two or more particular types of category data (e.g., users between the ages of 20 and 30 years old that undergo left knee arthroscopic surgery, users who weigh more than 300 pounds that are under the age of 20 years old, users who are taller than 7 feet that undergo hip replacement surgery using implant type ABC and model XYZ, or the like).

Any suitable input data (e.g., condition category data) may also be received or accessed (e.g., at operation 1308) for a particular POI and a particular POI event, such as any suitable sensor data indicative of any suitable biomechanical movement(s) of the POI sensed for any suitable period of time leading up to the particular POI event (e.g., the cadence values of the left foot, the right foot, and/or the average stride of the particular POI leading up to a particular POI event; the ground contact time values of the left foot, the right foot, and/or the average stride of the particular POI leading up to the particular POI event; the bounce values of the left foot, the right foot, and/or the average stride of the particular POI leading up to the particular POI event; any other suitable sensed biomechanical marker(s) of any suitable movement(s) of the particular POI leading up to the particular POI event; and/or the like) as well as any other suitable input data associated with the particular POI and/or with the particular POI event, including, but not limited to, any suitable demographic information about the particular POI (e.g., age of the particular POI, occupation of the particular POI, ethnicity of the particular POI, where the particular POI lives, etc.), any suitable health information about the particular POI (e.g., height of the particular POI, weight of the particular POI, any diagnosed physical ailments or conditions of the particular POI, any previous events (e.g., surgeries, and/or the like) experienced by the particular POI prior to the particular POI event, measured strength of one, some, or each limb and/or muscle group of the particular POI, and/or the like), any suitable information about the particular POI event (e.g., surgery or therapy type, implant type and/or implant model (if applicable), name of doctor that performed the particular POI event on the particular POI, name of hospital at which the particular POI event was performed on the particular POI, any medications and/or therapy administered during the particular POI event and/or to be administered after the particular POI event, and/or the like), and/or the like. Then at least one model (e.g., as trained at an operation 1306) may be used (e.g., at operation 1310) to predict an achievement of the particular POI for the particular condition of the particular POI for the particular POI as or based on an output of the model using the particular input data (e.g., condition category data) received or accessed (e.g., at operation 1308) for the particular POI and the particular POI event as input to the model, where such an output of the model may be any suitable data indicative of any suitable biomechanical movement(s) of the particular POI predicted to be made by the particular POI (e.g., predicted to be sensed) for any suitable period of time after the particular POI event (e.g., the predicted cadence values of the left foot, the right foot, and/or the average stride of the particular POI after the particular POI event; the predicted around contact time values of the left foot, the right foot, and/or the average stride of the particular POI after the particular POI event; the predicted bounce values of the left foot, the right foot, and/or the average stride of the particular POI after the particular POI event; any other suitable predicted biomechanical marker(s) of any suitable movement(s) of the particular POI after the particular POI event; and/or the like). Different models or different sets of models may be used to provide such output(s) for different types of input data (e.g., a model trained on input data for DU(s)/DU event(s) closely related to the input data for the particular POI/POI event (e.g., each model trained on DU input data for the same type of event as the particular POI event, and/or each model trained on DU input data for DU(s) with the same demographic information and/or the most similar health history information as the particular POI, and/or the like may be used to provide one or more predicted achievements for the particular POI/POI event).

Then, once at least one predicted achievement has been made for a particular POI and a particular POI event (e.g., data indicative of any suitable biomechanical movement(s) of the particular POI predicted to be made by the particular POI (e.g., data that may resemble the portion of one or more of the graphs of FIGS. 14-19 after the associated graph event or any other suitable predicted POI biomechanical movement data), it may be determined (e.g., at operation 1312) whether that predicted achievement satisfies at least one condition or rule (e.g., a pre-defined rule that may be associated with any suitable characteristic(s) of the POI and/or of the POI event and/or the like), and, if so, appropriate control data may be generated (e.g., at operation 1312) that may be used to control (e.g., at operation 1314) at least one functionality of at least one managed element. Any suitable predicted achievement data may be used to satisfy any associated rule in order to generate control data operative to control any suitable functionality of any suitable managed element (e.g., automatically without any active user (e.g., patient and/or caregiver) input following the prediction).

In some embodiments, a model used to predict the achievement of the particular POI for the particular POI event may be trained using successful recovery data related to successful DU recovery with respect to particular DU(s) and particular DU event(s) of the training data, such that the predicted achievement(s) provided by such a model using input data related to a particular POI and particular POI event may be indicative of a predicted recovery by the particular POI with respect to the particular POI event that may be most optimistic for success. Alternatively, a model used to predict the achievement of the particular POI for the particular POI event may be trained using all recovery data related to all DU recovery (e.g., successful, unsuccessful, and anywhere in between) with respect to particular DU(s) and particular DU event(s) of the training data, such that the predicted achievement(s) provided by such a model using input data related to a particular POI and particular POI event may be indicative of a predicted recovery by the particular POI with respect to the particular POI event that may be successful or unsuccessful. Therefore, a first type of trained model or set of trained models (e.g., successful recovery trained model(s)) may be used (e.g., at operation 1310) to predict how a successful recovery by the particular POI after the particular POI event may go, while a second type of trained model or set of trained models (e.g., all recovery trained model(s)) may be used (e.g., at another iteration of operation 1310) to predict how a recovery by the particular POI after the particular POI event may go, and those different predictions may be used with respect to different rules to potentially generate different control signals for controlling different functionalities of different managed elements. Therefore, in addition to or as an alternative to possibly predicting a successful recovery, the system may be used to predict fall risk or predict that a fall may occur after the event. After surgery, many patients are at high risk of falling because they are not accustomed to the effects of their surgery (e.g., on their gait). This system data may provide deeper insights into why a patient does or does not recover properly.

As an example, if a model (e.g., a model trained on only successful recovery data or on all recovery data) predicts particular recovery achievement for a particular POI/POI event and a comparison between that prediction and any actual recovery data that may be received for the particular POI/POI event (e.g., sensed by a sensor assembly for any suitable movement(s) of the POI post-event) indicates that the actual recovery is lagging or stalling compared to the predicted recovery by any suitable amount or in any suitable manner (e.g., the comparison satisfies a particular rule), then the system may generate a control signal to adjust any functionality of any managed element in any suitable manner, such as a control signal that may be operative to alert the POI and/or a caretaker (e.g., healthcare provider) thereof to schedule a clinical visit in order to address the negative comparison, and/or a control signal that may be operative to adjust or re-prioritize a recovery plan being followed by the POI for aiding in the recovery process (e.g., adding 25 squats to each recovery day's therapy regiment), and/or the like. Thus, comparison data indicative of a comparison between actual recovery data of a POI sensed by a sensor assembly of a system and a predicted recovery for the POI predicted by a model of the system can be used to help customize recovery exercises and plans, and/or such comparison data can be shared with physical therapists, thereby enabling deeper insight into patient mobility, which may help them modify or re-prioritize various exercises, stretches, or recovery treatments.

As an example, if a well-trained model predicts particular recovery achievement for a particular POI/POI event, that prediction may be used to provide the POI and/or the POI's caretaker(s) with an estimate of the amount of time the POI will need to achieve a full recovery after the POI event (e.g., based on the POI's demographic and/or health information, pre-event biomechanical gait signals of the POI, type of event, and the history of one or more DU's pre- and post-event biomechanical gait signals). This service can help provide more data for a patient to make an informed decision on the best time to schedule the surgery. As surgeries can take a long time to recover, patients may need to take time off from work or may miss an important event. Such model recovery prediction can help the patient, family, and physician identify an ideal time for surgery and post-surgery recovery.

Recovery prediction can also extend beyond pre-event and post-event outcome analysis and may help inform a patient and physician whether or not a surgery should even take place. In such embodiments, the system can be used as a diagnostic tool to determine whether a patient should be recommended surgery, and whether the surgery may improve their overall mobility and quality of life. In some cases, surgeries can be predicted to decrease overall quality of life and lead to decreased mobility and worse biomechanical gait baselines. Therefore, by using robust sensor assemblies and/or large scale data of pre- and post-event data for one or more DUs and at least pre-event data for a POI, model recovery prediction can be used to help estimate the potential overall improvement in quality of life for a POI. In some cases, this service can help a physician to determine that a particular event (e.g., a particular surgery) associated with a prediction is not needed, and other particular events may be used to generate other model recovery predictions to determine which of many possible events may be best suited for the POI (e.g., with respect to shortest recovery time or the like (e.g., with respect to a particular type of biomechanical marker or with respect to a combination of particular types of biomechanical markers)). In other cases, such recovery prediction can help identify the right surgery for a patient, as sometimes a patient may otherwise be prescribed the wrong surgery. In other cases, a particular surgery may potentially decrease the patient's mobility. In addition, some patients who are frail and weak may be put at greater risk due to the actual surgery itself, and predicted recovery may be used to determine in advance that a particular surgery should not be recommended for that patient.

In addition, machine learning models can also be built (e.g., using input data with DU biomechanical activity data sensed well in advance of an actual event) and used as an “early detection” diagnostic to detect early on if a patient may require surgery in the well-off future (e.g., not imminently but potentially years from now). This may be important to identify patients who may need to get surgery while they are still healthy, strong, and can benefit from the increased mobility later in life. Some patients may not recognize that they need surgery until it is too late. This often leads to decreased mobility while they are delaying an inevitable surgery. Reduced mobility can lead to other health complications, such as diabetes and obesity. Identifying patients who will eventually need surgery early on can result in long term health benefits and potential reductions in healthcare costs.

As mentioned, in addition to or as an alternative to predicting a successful recovery for a POI/POI event, the system may be used to predict fall risk or detect when a fall has occurred. After surgery, many patients are at high risk of falling because they are not accustomed to the effects of their surgery on their gait. This data may provide deeper insights into why a patient does or does not recover properly. For example, sensors on the foot and/or pelvis can measure the biomechanical characteristics that are highly correlated with falls. Some such metrics may include, but are not limited to, cadence, cadence variability, step length, step length variability, step width variability, stride speed, foot vertical displacement, toe clearance, foot swing time, double stance time, pelvic lateral displacement, pelvic coronal drop, and pelvic transverse rotation. If any one or multiple actual detected biomechanical achievement signals (e.g., of actual POI post-event data) are detected (e.g., at operation 1312) as abnormal (e.g., compared to appropriate predicted post-event data (e.g., for satisfying any suitable rule (e.g., at operation 1312))), the system may be configured to alert the user (e.g., at operation 1314) to pay attention to walking or ask the patient to sit down and/or to give the patient an objective score (e.g., to help him/her quantify their fall risk at that particular moment, or over time, etc.), and/or the system may be configured to generate any suitable actuator assembly control data that may be operative to control (e.g., at operation 1314) any suitable actuator assembly worn by the POI to help assist and/or support the POI in order to help avoid the fall at risk of occurring.

Additionally or alternatively, a care provider can be alerted to fall risk or if a fall has been detected. These indicators can also provide additional context to why a patient may not be recovering properly or as quickly as projected. Additional details with respect to certain types of fall prediction may be described in U.S. Patent Application Publication No. 2018-0177436, titled “BIOMECHANICAL PLATFORM FOR REMOTE MONITORING FOR ELDERLY FALL PREDICTION, DETECTION AND PREVENTION,” as published on Jun. 28, 2018, which is hereby incorporated by reference herein in its entirety.

While the system may be configured to track the progress of walking gait mobility, it can also provide real-time feedback via audio, haptic, or any other communication medium to correct a patient in real-time and provide coaching and personalized tips to accelerate recovery or gait retraining outside a clinic. For example the system may define a personalized recovery plan or training plan that may prioritize recommended adjustments that it may determine ought to be made based on the patient's mobility status, the progress of the user, ease of learning, feedback from a physician, and/or predicted recovery achievement.

For example, if the patient was determined to have an unstable pelvis while walking pre-event and/or post-event, the system can suggest pelvic drop be prioritized first until the patient has mastered it before moving onto another biomechanical gait marker to work on during post-event recovery (e.g., by providing recommendations and/or actuator control based on comparison between actual post-event achievement data for that biomechanical gait marker and predicted post-event achievement data for that biomechanical gait marker (e.g., as predicted based on models trained with successful DU recovery data and/or all available DU recovery data and/or based on actual recovery data of comparable DUs from comparable events). In another example, patients can be reminded by the system when their cadence variability increases significantly, which may be another sign of gait instability and potential fall risk. The system may then coach the user to focus on cadence and ask the user to walk according to a predefined cadence, such as 60 steps per minute or any other suitable cadence that may be identified using any suitable predicted recovery data. The system may provide feedback, such as a beep, every second similarly to a metronome to help the patient keep their rhythm while they walk.

The personalized coaching can also include additional exercises or stretches to strengthen specific muscles and reduce overall instability. For instance, if the patient is determined to be walking asymmetrically on his right side post-event (e.g., as compared to predicted recovery data or actual recovery data of comparable DUs from comparable events), the system may suggest exercise(s) to activate and strengthen the patient's left leg to begin balancing the stride asymmetry.

The system can also be personalized to work with the specific physical therapists (PT) or their gait retraining plans (e.g., at operation 1312 when determining whether one or more rules have been satisfied for generating certain control data). The system can focus on the PT's priorities, provide customized feedback from the PT, and send the progress updates directly to the PT. The system can be configured to send all this information back to the PT who can modify a training program virtually depending on the patient's progress (e.g., as compared to predicted recovery data or actual recovery data of comparable DUs from comparable events). With this new deeper information, the PT may be empowered to make decisions without having to see the patient in his clinic. The PT can then focus his time on the patients who may need it most. Additionally or alternatively, the system may be configured to modify a training or recovery program automatically (e.g., at operations 1312 and 1314) without having to ask the PT.

A sensor assembly worn or otherwise carried by a user in any suitable manner (e.g., on the pelvis) may be configured to compute any suitable biomechanical gait signals of the user (e.g., for any biomechanical movements 630 of FIG. 6, or for particular biomechanical movements, such as cadence, vertical displacement of the pelvis, horizontal velocity of the pelvis, pelvic transverse rotation, pelvis tilt, and/or pelvic drop) with every step that the user may take (e.g., while running or walking) and such biomechanical signals may be utilized by the system along with any suitable demographic and/or health and/or other suitable type(s) of user information to attempt to determine or predict a step length of the user. For example, a biomechanical model (e.g., a regression model) may be built that may use one or more various biomechanical gait metrics as inputs and step size as outputs (e.g., for training and/or for predicting step size). The data may include gait metrics computed at every step or averaged over a number of steps taken by a user. The system may provide a mobile application that a user can walk or run with to get coaching and real-time feedback on various biomechanical attributes, including, but not limited to, their posture, walking gait, or running gait. With a smart phone, an application may include receiving GPS data (e.g., periodically) from an enabled GPS assembly to measure distance traveled by the user. Gait metrics may be synchronized with such GPS data to compute step size and serve as training and/or testing data. Multiple approaches may be taken for estimating a running step length and running gait speed of an individual. However these same approaches may be used to estimate a walking step length and walking gait speed of an individual. Step length may be the forward displacement of each foot. A similar or alternative metric, stride length, may be the forward displacement of two consecutive steps of the right and left foot. Step length may be along the axis of dominant motion of a user. The step direction or angle may additionally be measured. In some cases, there may be a true step length measuring the distance from a reference point of the body to the foot positions. Alternatively, there may be an effective step length that measures the effective distance a step moves a user if, for example, the user walks with a waddle or with steps not in a parallel direction. Gait speed may be the measure of the speed of steps or strides. Alternatively or additionally, stride/step duration and/or other biomechanical signals may be generated.

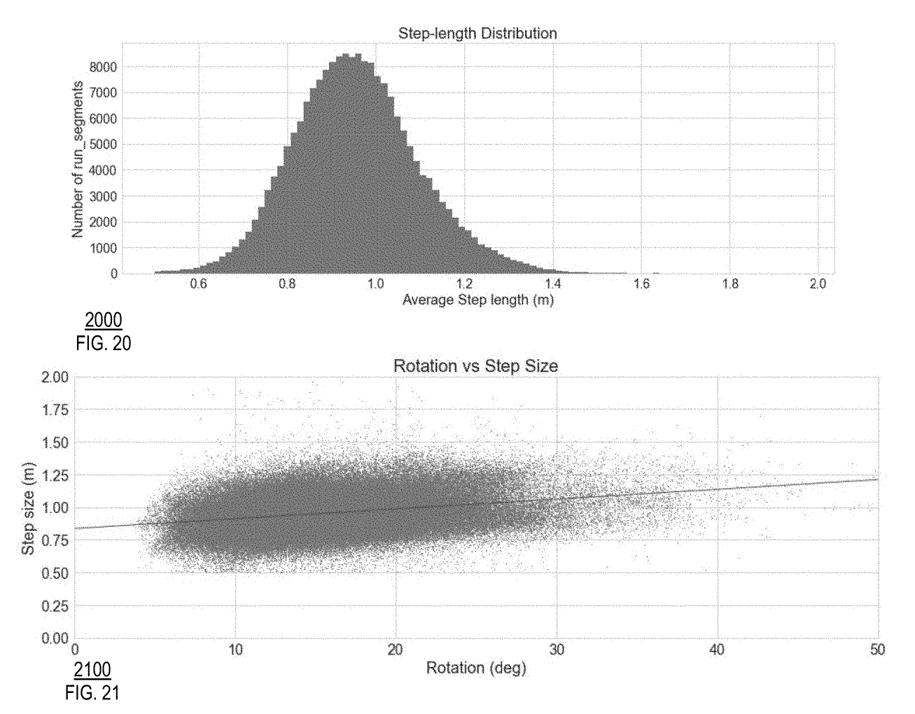

To compute running step length, running biomechanical gait and GPS data from thousands of runners may be analyzed and cut into segments of continuous running. Running metrics may be averaged and summed over a run or walk segment. Segments of less than 50 steps or any other suitable minimum number of steps may be discarded. For example, as shown by graph 2000 of FIG. 20, a distribution of average step size (e.g., in meters) may be plotted for various such run segments. The shape of the plot may be very close to a normal distribution, while the average step-size may be 0.95 meters (e.g., just a little over a yard) and the standard deviation may be 0.15 meters (e.g., about 6 inches). Runners considered in this dataset may span a wide demographic (e.g., users with ages ranging from 14 years to 84 years, with weights ranging from 90 pounds to 300 pounds, heights ranging from 4′8″ to 6′8″, and with average speeds ranging from 2.9 miles per hour to 9.5 miles per hour). Graph 2100 of FIG. 21 may highlight the positive correlation between average step size (e.g., in meters) and pelvic transverse rotation (e.g., in degrees) of the same runner data. with a correlation coefficient of 0.0075 (e.g., as may be shown by the slope of the best fit line), whereby a simple linear regression may suggest that, on average, a change of 1 degree of rotation can affect the step size by 7.5 millimeters.

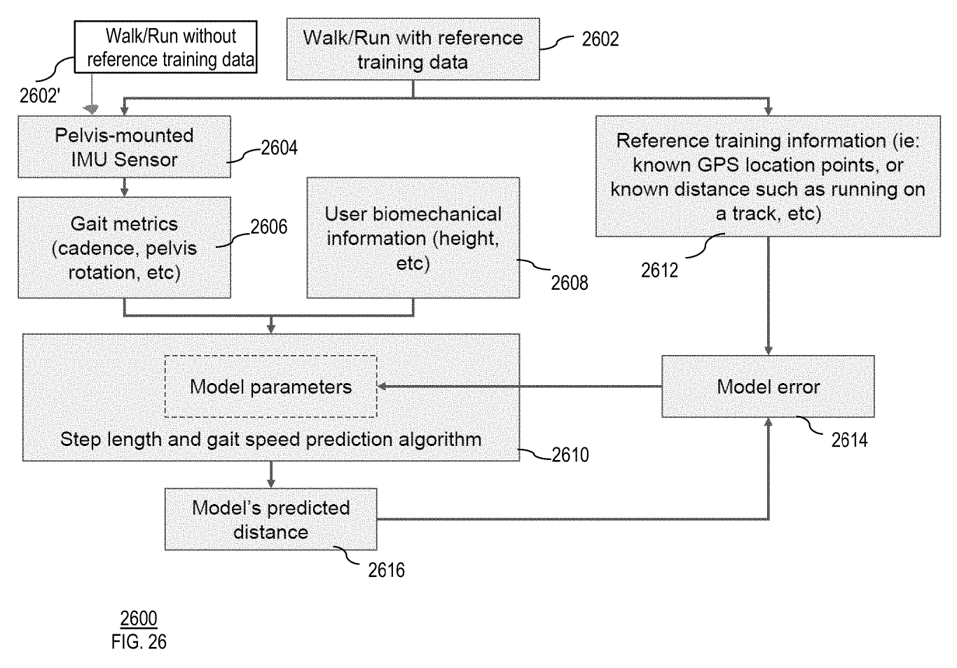

In one approach, a model (e.g., a multi-variable linear regression model) may be built to predict the step-size from the above running gait metrics. After training (e.g., using 5-fold cross-validation, and L1 regularization) a model (e.g., at operation 2610 of process 2600 of FIG. 26) using the sensed running biomechanical gait data as input data (e.g., at operation 2606 from any suitable sensors at operation 2604 for any suitable user(s) generating reference training data at operation 2602 of process 2600 of FIG. 26) and the GPS detected or otherwise entered actual achievement distance traveled data as output data (e.g., at operation 2612 of process 2600 along with any (e.g., feedback fed) model error of operation 2614), the model may be determined to predict a distance (e.g., at operation 2616 of process 2600) that may achieve a root-mean-square (RMS) deviation or test error of 0.11 meters or any other suitable model error (e.g., operation 2610 may train a model/algorithm using known distance points for a given walk/run from operation 2612 and the gait metrics and biomechanical information of operations 2606 and 2608 may be used, along with the model parameters, to create a prediction of the distance at operation 2616, where the error between the model's prediction and the reference distance points may be used to tune the model parameters (e.g., at operation 2614) to iteratively obtain better predictions of the distance). Compared with the average 0.95 meter step-size, this may be about a 12% error. This may be equivalent to an error of half a lap around a track for every 4 laps (e.g., every mile) run. In practice, GPS data that may be detected by a smart phone may have about a 3% to 6% error while GPS data that may be detected by a smart watch may have about a 1% to 3% error. In aggregate, the metrics of such a model may be reasonable predictors of step-size, but its accuracy may greatly improve when evaluated on an individual user by individual user basis. For example, each individual could have a tight correlation between their personal gait metrics and step-size, but since each person might be different from the next, in aggregate, the correlation may be weaker.

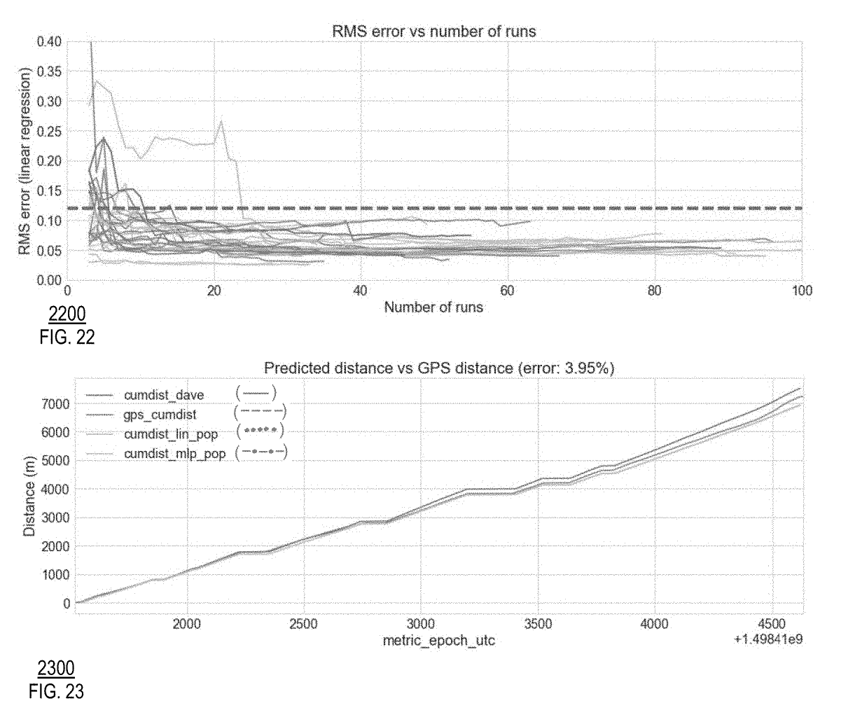

Thus, another approach may be to build at least one regression model that is unique to each individual user. In which case, a pertinent question to answer may be “how many runs does a runner need to accumulate before a personalized model for that runner may become most accurate?”. The more runs a runner accumulates, the more data that may be collected, and, therefore the more accurate a model may be for that user. For example, this may be simulated by ordering runs chronologically for a runner (e.g., n total runs) and then training a model (e.g., a linear regression model) on the runner's first k runs (e.g., where k<n) and then testing the model on all remaining n-k runs. When different models for various different runners with n total runs may be trained for each k that is greater than or equal to 3, the resulting test error for each k number of runs may be plotted as shown by graph 2200 of FIG. 22 (e.g., where each runner may be represented by a different plotted line). For example, the x-axis value of each plotted line/runner may show the first k runs the model is trained on and the y-axis value of each plotted line/runner may show the RMS error when the model is applied to remaining n-k runs (e.g., all runs in the future). The error may be shown to converge after about 10 runs for most runners. Approaches can be taken to deal with edge cases, such as users where the model converges after 22 runs. In the end, as shown, all the models may converge to test errors below 10%, which may be below the general model error of 12%, and, on average, as shown, the individual models may converge to an error of 5%.

One approach to minimizing the prediction error during the first 10 runs may be to apply the general population model to the first 10 runs and then apply the user-specific model to subsequent runs. Alternatively, a model can be trained on different segments of the running population based on demographic, running type, style, running expertise, and/or any other suitable user-describing input data (e.g., category data), where such a model may potentially be more accurate than the general population model but can also be pre-trained and used for the first 10 runs. Both models may become more accurate with more running data. After these first 10 runs, the model may switch to the individual-user model. Another approach may be to start with a demographic model and re-train it with user-specific runs to improve the accuracy.

One of the pitfalls of using RMS error as a metric for performance when predicting total running distance may be that if a model over-predicts or under-predicts running distance, the RMS metric may treat both predictions the same and may add up the magnitude of the errors. However, because step counts may be added over the length of a run, it is possible for these errors to cancel out and add up to a smaller net error, thereby resulting in a more accurate estimate of total running distance. This can be described by the triangle inequality of the following equation:

$$\vert {\sum }_{i} ( {\hat{x}}_{i} - {x}_{i} ) \vert <= {\sum }_{i} \vert {\hat{x}}_{i} - {x}_{i} \vert ,$$

where \hat{x}ᵢ may be the predicted step length and xᵢ may be the step length computed from GPS data. This equation may help explain that the sum of the errors over all steps i taken in a run (e.g., left hand side of the equation) can be less than or equal to the sum of the absolute errors (e.g., right hand side of the equation). As shown by graph 2300 of FIG. 23, an accumulated distance over the course of a run determined by actual achievement distance data (e.g., metric-epoch-utc) may be plotted against predicted estimated distance data (e.g., meters) output from various models and GPS data, which may include a linear regression model that is trained on a specific user (e.g., the plotted data for “cum_dist_dave” with a blue or full line), a linear regression model that is trained on a population of runners (e.g., the plotted data for “cum_lin_pop” with a green or dotted line), a multi-layer perceptron model (e.g., neural net) that may be trained on a population of runners (e.g., the plotted data for “cumdist_mlp_pop” with a yellow or an alternating dotted/dashed line), and a GPS data output (e.g., the plotted data for “gps_cumdist” with a red or dashed line). Although the GPS data may have a 3% to 5% error, it may be assumed that model accurately models the relationship between metrics and step length, such that, if the model is applied to an accurate distance measure, similar performance may be achieved. A same approach may be applied to other types of regressions, such as least squares regression, Bayesian linear regression, kernel regression, stochastic gradient descent non-linear regression, decision trees regression, spline regression, support vector regression, and/or deep-nets regression.

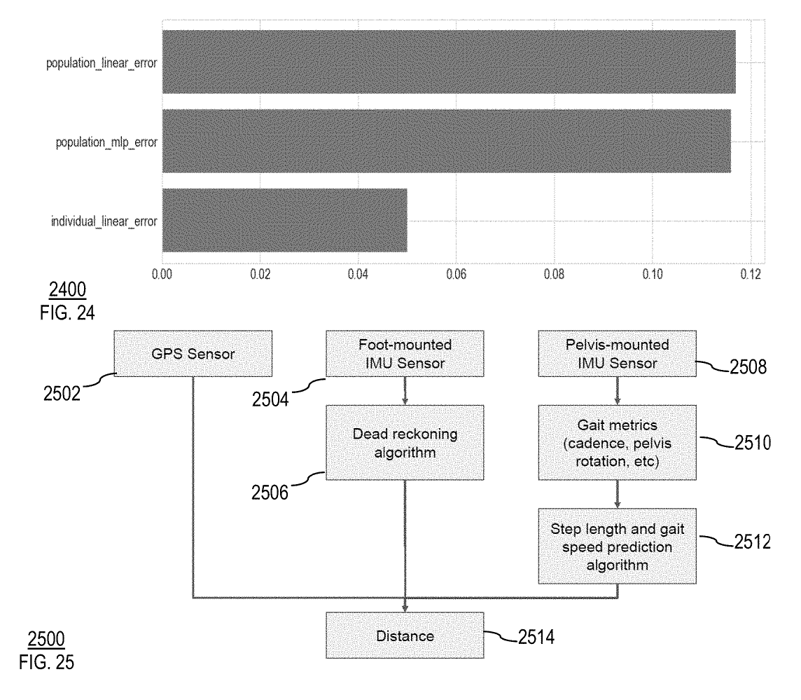

As shown, all such models may behave similarly, but more strikingly, they may all perform with an accuracy of close to 4% by the end of the run. Each model may have about a 10% error from step to step, but, because the errors can cancel themselves out, the overall accuracy over an entire run is only 4%, which may be comparable to GPS error. Therefore, as shown by graph 2400 of FIG. 24, a comparison of the test errors between a linear regression model trained on the population, a multiple-perceptron model trained on the population, and a linear regression model trained on an individual user may be provided.

A more sophisticated machine learning model may not perform better than linear regression, potentially due to the high quality and high-dimensionality to the biomechanical data. In summary, a single sensor worn on the pelvis of a user can be used to quantify a plurality of biomechanical gait metrics. These gait metrics can be inputted into a machine learning model that can predict distance traveled at or near the same error of a GPS device. The model can estimate the step length for the right leg only, left leg only, or over the entire stride (e.g., left and right step). Once step length has been computed, gait speed can be estimated by dividing the length of the step over the time it took to complete the step, where such time data may be included in the sensed gait metric data.

The system's ability to predict high accuracy step length and gait speed can power a new generation of research and applications. In addition, the system can enable products and solutions to calculate step length and gait speed without the need of a GPS device or component. This may be advantageous because the system may consume less power and can be used in locations where GPS wouldn't work. GPS assemblies are often power-hungry component that can have material impact on a system's (e.g., user subsystem's) form-factor, size, and/or battery capacity. Therefore, a system operative to accurately predict step length and/or gait speed without GPS data for a particular POI may enable system designers to design smaller and thinner devices because the system may require less battery capacity. In addition, GPS may not work everywhere (e.g., GPS may have difficulty working indoors, in underground cave systems, and/or around other natural formations that can attenuate the GPS signal).

A system may be configured to combine magnetometer data (e.g., compass data and/or any other suitable non-GPS data indicative of direction(s) travelled over time (e.g., direction and time values)) with predicted step length and step count data to estimate navigational pathways traversed through time and space. These pathways can be used to estimate real-time locations of people indoors or chart out pathways of people movement. For example, the system may be used to automatically map out underground cave systems for explorers, or help them find their way back out if they get lost. In another example, the system may be used to map out the flow of human traffic in a construction site, warehouse, indoor shopping mall, or other busy work environment. The system can also be used in conjunction with GPS, Wi-Fi, 2G/3G/4G/LTE, Bluetooth beacons, and/or other technologies that can help the system to calibrate location data and provide higher location resolution.

Step length and gait speed may be two biomechanical gait markers that may be important to addressing a number of specific applications. In particular, these metrics have been observed to be correlated with issues such as fall risk, post-surgical recovery, and movement disorder severity. Therefore, these metrics can be used as inputs into other models that may have been trained to predict fall risk, post-surgical recovery rate, movement disorder severity, and/or treatment efficacy. Some examples of such applications may now be described.

In a running context, predicted step length and gait speed for a POI (e.g., at operation 1310 or at operations 2610 and 2616 using biomechanical metrics of the POI determined at operations 260T and 2604 and 2606 or at operation 1308 and a model trained at operation 1306) can be used (e.g., at operations 1310 and 1312) to help the POI runner track its training progress and estimate its pace time and distance traveled without the need to carry a GPS device. Measuring the variance of these metrics (e.g., along with other biomechanical metrics) over a run or number of runs can also help measure or predict the onset of fatigue as a user trains. If the variance is determined (e.g., at operation 1312) to increase significantly over a run, the runner may be alerted to slow down or take a break (e.g., at operation 1314). These metrics can help the user or a coach track and modify a training plan to help the runner run safer, further, and/or faster. If a stride length or gait speed variance is determined (e.g., at operation 1312) to increase significantly, the system can provide real-time feedback (e.g., at operation 1314) to the user to be more mindful while running, to focus on improving a specific metric, or to alert the user if they are at risk of fatiguing.

Both gait speed and step length may be important biomechanical metrics for walking gait analysis. The system can monitor a predicted walking step length, step length variability, gait speed, and/or gait speed variability. These biomechanical markers may be important and highly correlated with predicting fall risk, post-recovery of a surgery, and/or disease severity for various movement disorders. For example, these metrics can be used to measure, monitor, and/or predict (e.g., at operation 1312) when a user may be at high risk of falling. If such a risk is detected (e.g., at operation 1312), a care provider can be alerted (e.g., at operation 1314). The system can additionally or alternatively notify (e.g., at operation 1314) the user by voice, text, or through another connected internet of things (IoT) device to take a rest, sit down, drink water, and/or avoid strenuous activity. The system can also send (e.g., at operation 1314) an alert to the user's loved ones or a nurse call center who can call the user directly and make sure the user is alright.

Some research has observed correlations in various biomechanical gait markers and Parkinson's Disease (PD) severity. Metrics, such as larger step length, faster gait speed, faster step cadence, and/or low cadence variability are among metrics that have been correlated with less severe Parkinson's Disease behaviors, while shorter step lengths, slower gait speed, slower cadence, and/or higher cadence variability are among metrics that have been observed with patients with more severe PD. As PD behavior fluctuates, the system may be configured to accurately and comprehensively characterize PD behavior over days and weeks or any other suitable time period(s). This information can be used by physicians to understand the full scope of how PD impacts a patient, the efficacy of various PD drug prescriptions, and/or may help accelerate the time to stabilize drug dosages. Additionally or alternatively, the system can be configured to provide data (e.g., at operation 1314) that may be operative to help physicians, pharmaceutical companies, and other suitable caretakers understand the effects of various drug treatments beyond PD (e.g., to all diseases that may impact mobility), such as pain, obesity, arthroplasty, and other diseases. For instance, this system can be used to measure the pre- and/or post-mobility changes of a patient suffering from pain that takes a pain relief drug or goes through a physical therapy session (e.g., an event of a condition of process 1300).

Step length and gait speed, among additional biomechanics, can also be used to evaluate the pre- and/or post-surgical recovery of a patient that may undergo a surgery, such as a knee, hip, or ankle arthroplasty. A sensor assembly of the system can be worn for a few days before the surgery to establish a baseline. The sensor assembly can then be worn after surgery for a period of time to measure and monitor the general recovery pattern. As a patient recovers, step length and gait speed may usually increase. If the patient fails to recover properly (e.g., as may be determined by comparison with known achievements of other similar users or by comparison with a predicted achievement for the patient by any applicable model(s), the system can detect this and alert a care provider to check in with the patient or can generate any suitable control signal for adjusting any other suitable functionality of any suitable managed element in any suitable manner.

The system may be configured to estimate the location and pathway a user has traveled, such that, if a user is detected to have fallen down, the system may be configured not only to send out an alert to an emergency system indicative of the occurrence of the fall, but also an estimated location of the fall (e.g., based on the number of steps and distance traversed (e.g., as may be predicted by any suitably trained model) as well as the direction traveled (e.g., the direction as may be monitored with a magnetometer or other suitable sensor assembly of a user subsystem)). Similar to other types of position dead-reckoning solutions (e.g., following a first path of components 2504 and 2506 and 2514 of a process 2500 of FIG. 25) or GPS solutions (e.g., following a second path of components 2502 and 2514 of process 2500), such a system may be configured to perform real-time user location when indoors, underground, or where GPS signals may be difficult to receive using body-mounted sensor(s) (e.g., pelvis-mounted IMU sensors) and gait metrics (e.g., cadence, pelvis rotation, etc.) as may be detected by such sensor(s) and any suitable step length and/or gait speed prediction algorithm(s) and/or model(s) for determining a distance traveled without GPS data (e.g., following a third path of components 2508, 2510, 2512, and 2514 of process 2500 of FIG. 25).

The system may be configured to monitor workers who may work in harsh and unsafe conditions. For example, the system can monitor construction workers who may get injured, fall, or be rendered unconscious in a work site, tunnel, or other large area. The system can send out an estimated location periodically to a central system that can monitor for potential injury or emergency or understand traffic flow of people. The system can also mark estimated locations automatically where a worker is detected to have been injured, falls, or loses stability. A central subsystem (e.g., cloud server or otherwise) of the system can be configured to collect all this data across multiple sensor assemblies and identify unsafe zones and/or hotspots for optimizing the safety conditions of a work environment. If a worker approaches such a hotspot, the system can be configured to alert the worker with caution. If a worker is determined to be walking or running too quickly through a hotspot, the worker can also be alerted to slow down (e.g., by the worker's own user subsystem or a nearby auxiliary subsystem). Additionally or alternatively, the system can be configured to highlight other hotspots in any other environment. For example, the system can be used to remotely monitor elderly parents who live at home and mark any potential hotspots in the home where a user may have lost balance, tripped, or fallen down. These hotspots can be alerted to the elderly user and/or any suitable caretaker or family member. Alerts can also automatically be triggered by the system when a user comes near such a hotspot. In the case of when an elderly parent has fallen, the system can be configured to alert emergency personnel of the type of the accident, severity of the accident, and/or location of the accident, thereby making it easier for emergency responders to react. In the context of a home environment, the system may be more suitable for location tracking as it may have higher resolution than GPS signal tracking inside such home environments.

For patients who may suffer from Alzheimer's or who may frequently wander off, the system can be configured to keep the patient from wandering too far. For example, if the system determines that a patient has wandered too far away from home, hospital, or nursing home or other location of interest, the system may be configured to automatically alert a family member or nursing staff or other suitable caretaker of the event and location. The system can additionally or alternatively alert the user and provide audio feedback and directions to the user for finding their way back home.

The system can be used to monitor worker productivity. For example, the system may be configured to measure the movement of construction workers or delivery personnel. The paths a delivery personnel or other worker may take can be measured (e.g., based on predicted step length and/or gait speed) and used to optimize a worker's schedule or delivery path. The system can also be used to monitor the productivity of janitor staff. The system can measure the overall motion, mobility, and aggregate the amount of surface area a cleaning staff covers during their shift. The data can be aggregated and used to highlight potential areas that are skipped, or may need attention in the future.

In addition to determining a user's location, health status, and/or productivity, the system may be configured to estimate physical attributes of the user, such as height, weight, and flexibility. These attributes can be estimated (e.g., at operations 1312 and 1314) as there may be relationships (e.g., rules) associated with and between step length, gait speed, and/or other biomechanical gait metrics and user height, flexibility, and/or weight that may be used to determine such estimates based on certain predicted biomechanical gait metrics.

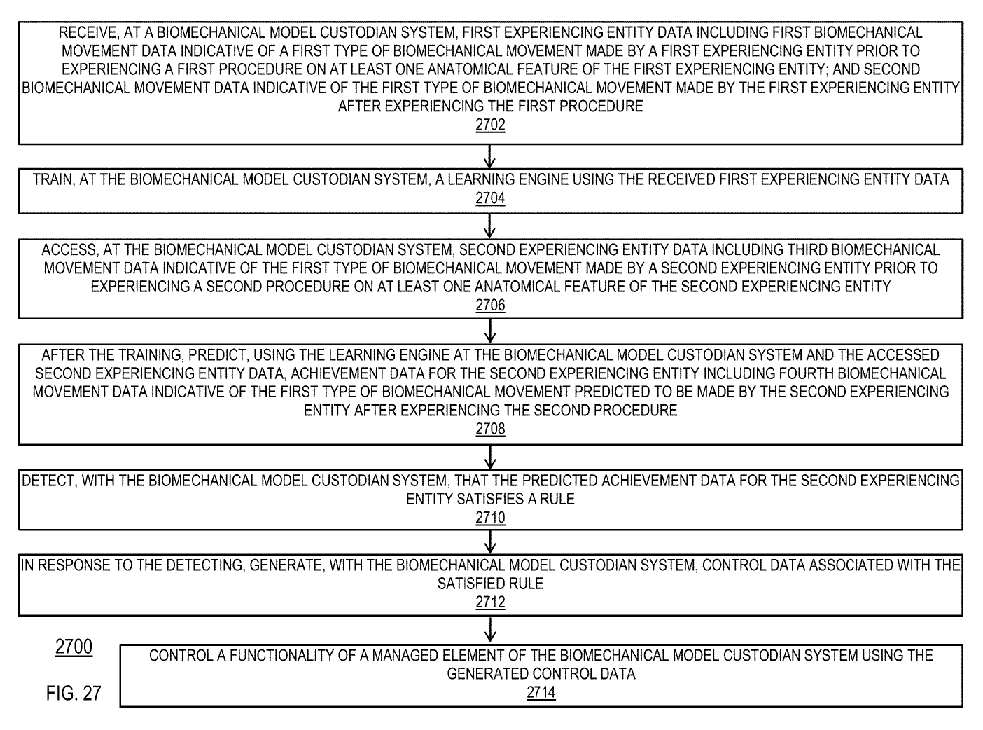

FIG. 27 is a flowchart of an illustrative process 2700 for managing biomechanical achievements using a biomechanical model custodian system (e.g., system 1). At operation 2702 of process 2700, the biomechanical model custodian system may receive first experiencing entity data that may include first biomechanical movement data indicative of a first type of biomechanical movement made by a first experiencing entity prior to experiencing a first procedure on at least one anatomical feature of the first experiencing entity, and second biomechanical movement data indicative of the first type of biomechanical movement made by the first experiencing entity after experiencing the first procedure (e.g., pre-recovery biomechanical data and recovery biomechanical data for a DU). At operation 2704 of process 2700, the biomechanical model custodian system may train a learning engine using the received first experiencing entity data (e.g., system 1 may train device biomechanical model 1105a or auxiliary comfort model 1255a). At operation 2706 of process 2700, the biomechanical model custodian system may access second experiencing entity data that may include third biomechanical movement data indicative of the first type of biomechanical movement made by a second experiencing entity prior to experiencing a second procedure on at least one anatomical feature of the second experiencing entity (e.g., pre-recovery biomechanical data for a POI). After the training of operation 2704, at operation 2708 of process 2700, the biomechanical model custodian system may predict, using the learning engine and the accessed second experiencing entity data, achievement data for the second experiencing entity including fourth biomechanical movement data indicative of the first type of biomechanical movement predicted to be made by the second experiencing entity after experiencing the second procedure (e.g., predict recovery biomechanical data for the POI). At operation 2710 of process 2700, the biomechanical model custodian system may detect that the predicted achievement data for the second experiencing entity satisfies a rule. In response to the detecting of operation 2710, at operation 2712 of process 2700, the biomechanical model custodian system may generate control data associated with the satisfied rule. At operation 2714 of process 2700, a functionality of a managed element of the biomechanical model custodian system may be controlled using the generated control data.

It is understood that the operations shown in process 2700 of FIG. 27 are only illustrative and that existing operations may be modified or omitted, additional operations may be added, and the order of certain operations may be altered.

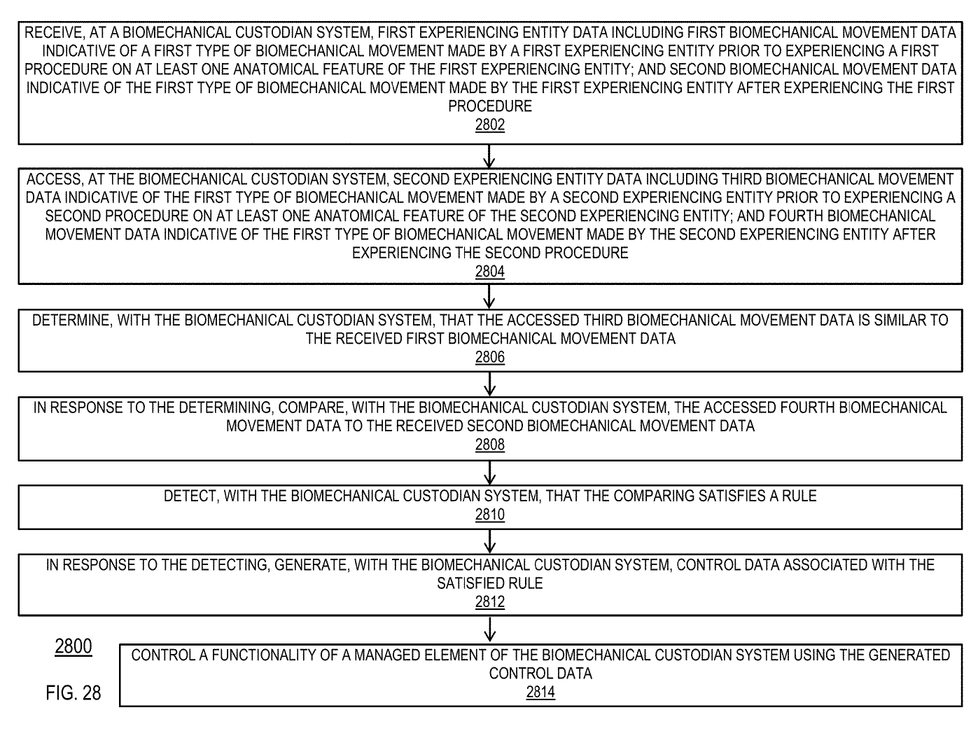

FIG. 28 is a flowchart of an illustrative process 2800 for managing biomechanical achievements using a biomechanical custodian system (e.g., system 1). At operation 2802 of process 2800, the biomechanical custodian system may receive first experiencing entity data that may include first biomechanical movement data indicative of a first type of biomechanical movement made by a first experiencing entity prior to experiencing a first procedure on at least one anatomical feature of the first experiencing entity, and second biomechanical movement data indicative of the first type of biomechanical movement made by the first experiencing entity after experiencing the first procedure (e.g., pre-recovery biomechanical data and recovery biomechanical data for a DU). At operation 2804 of process 2800, the biomechanical custodian system may access second experiencing entity data including third biomechanical movement data indicative of the first type of biomechanical movement made by a second experiencing entity prior to experiencing a second procedure on at least one anatomical feature of the second experiencing entity, and fourth biomechanical movement data indicative of the first type of biomechanical movement made by the second experiencing entity after experiencing the second procedure (e.g., pre-recovery biomechanical data and recovery biomechanical data for a POI). At operation 2806 of process 2800, the biomechanical custodian system may determine that the accessed third biomechanical movement data is similar to the received first biomechanical movement data (e.g., by determining that a baseline of the accessed third biomechanical movement data is within a first particular threshold of a baseline of the received first biomechanical movement data (e.g., that the baselines are similar to each other by at least a particular degree as may be determined in any suitable manner using any suitable computing technique(s))). In response to the determining of operation 2806, at operation 2808 of process 2800, the biomechanical custodian system may compare the accessed fourth biomechanical movement data to the received second biomechanical movement data (e.g., to detect any similarities or differences therebetween). At operation 2810 of process 2800, the biomechanical custodian system may detect that the comparing satisfies a rule. In response to the detecting of operation 2810, at operation 2812 of process 2800, the biomechanical custodian system may generate control data associated with the satisfied rule. At operation 2814 of process 2800, a functionality of a managed element of the biomechanical custodian system may be controlled using the generated control data. In some embodiments, the determining of operation 2806 may include determining that a baseline of the accessed third biomechanical movement data is within a first particular threshold of a baseline of the received first biomechanical movement data, the comparing of operation 2808 may include identifying that a baseline of the accessed fourth biomechanical movement data is more than a second particular threshold off from a baseline of the received second biomechanical movement data, and/or the detecting of operation 2810 may include recognizing that the identifying satisfies the rule.

It is understood that the operations shown in process 2800 of FIG. 28 are only illustrative and that existing operations may be modified or omitted, additional operations may be added, and the order of certain operations may be altered.

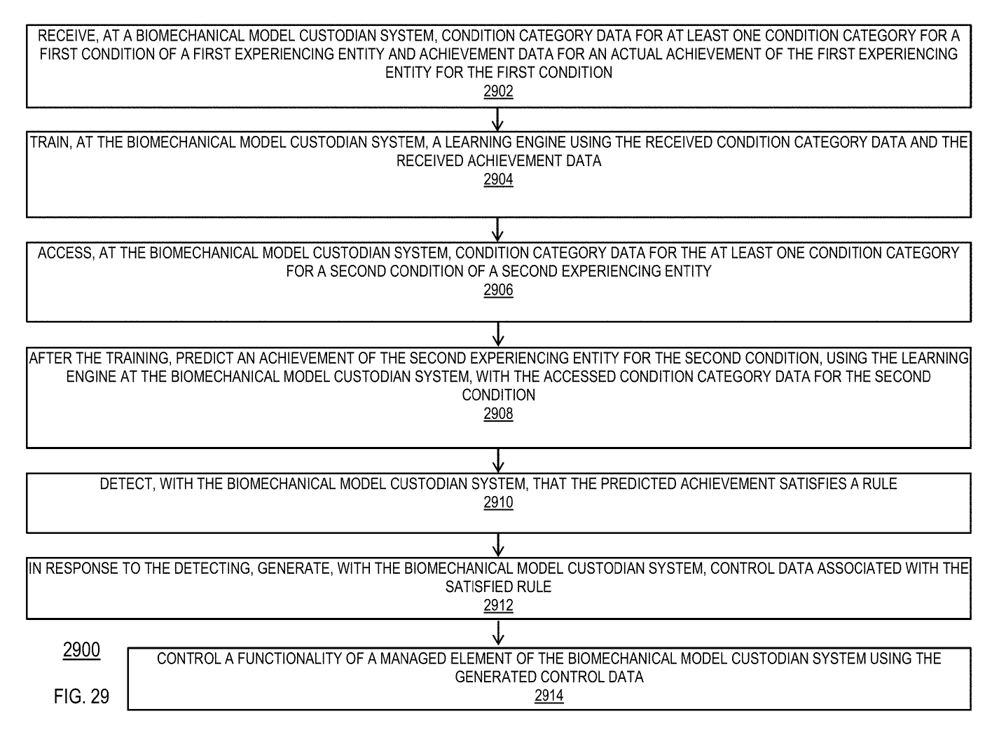

FIG. 29 is a flowchart of an illustrative process 2900 for managing biomechanical achievements using a biomechanical model custodian system (e.g., system 1). At operation 2902 of process 2900, the biomechanical model custodian system may receive condition category data for at least one condition category for a first condition of a first experiencing entity and achievement data for an actual achievement of the first experiencing entity for the first condition (e.g., pre-recovery biomechanical data and recovery biomechanical data for a DU). At operation 2904 of process 2900, the biomechanical model custodian system may train a learning engine using the received condition category data and the received achievement data (e.g., system 1 may train device biomechanical model 1105a or auxiliary comfort model 1255a). At operation 2906 of process 2900, the biomechanical model custodian system may access condition category data for the at least one condition category for a second condition of a second experiencing entity (e.g., pre-recovery biomechanical data for a POI). After the training of operation 2904, at operation 2908 of process 2900, the biomechanical model custodian system may predict, using the learning engine at the biomechanical model custodian system, with the accessed condition category data for the second condition, an achievement of the second experiencing entity for the second condition (e.g., predict recovery biomechanical data for the POI). At operation 2910 of process 2900, the biomechanical model custodian system may detect that the predicted achievement satisfies a rule. In response to the detecting of operation 2910, at operation 2912 of process 2900, the biomechanical model custodian system may generate control data associated with the satisfied rule. At operation 2914 of process 2900, a functionality of a managed element of the biomechanical model custodian system may be controlled using the generated control data.

It is understood that the operations shown in process 2900 of FIG. 29 are only illustrative and that existing operations may be modified or omitted, additional operations may be added, and the order of certain operations may be altered.

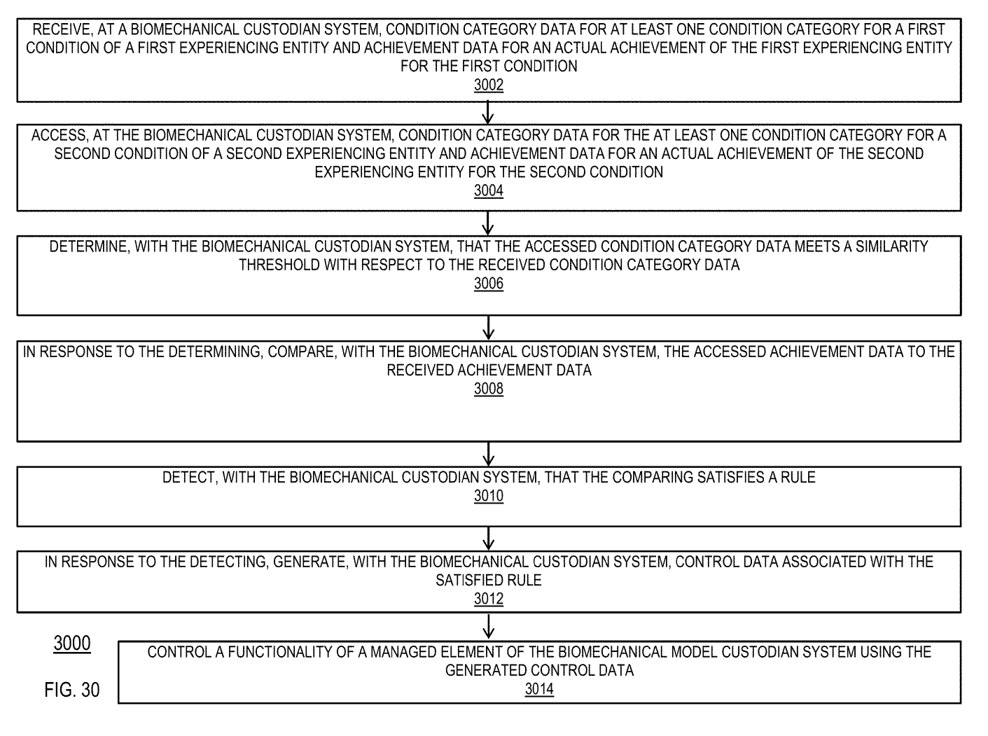

FIG. 30 is a flowchart of an illustrative process 3000 for managing biomechanical achievements using a biomechanical custodian system (e.g., system 1). At operation 3002 of process 3000, the biomechanical custodian system may receive condition category data for at least one condition category for a first condition of a first experiencing entity and achievement data for an actual achievement of the first experiencing entity for the first condition (e.g., pre-event data and post-event data for a DU). At operation 3004 of process 3000, the biomechanical custodian system may access condition category data for the at least one condition category for a second condition of a second experiencing entity and achievement data for an actual achievement of the second experiencing entity for the second condition (e.g., pre-event data and post-event data for a POI). At operation 3006 of process 3000, the biomechanical custodian system may determine that the accessed condition category data meets a similarity threshold with respect to the received condition category data (e.g., by determining that a baseline of the accessed condition category data is within a first particular threshold of a baseline of the received condition category data (e.g., that the baselines are similar to each other by at least a particular degree as may be determined in any suitable manner using any suitable computing technique(s))). In response to the determining of operation 3006, at operation 3008 of process 3000, the biomechanical custodian system may compare the accessed achievement data to the received achievement data (e.g., to detect any similarities or differences therebetween). At operation 3010 of process 3000, the biomechanical custodian system may detect that the comparing satisfies a rule. In response to the detecting of operation 3010, at operation 3012 of process 3000, the biomechanical custodian system may generate control data associated with the satisfied rule. At operation 3014 of process 3000, a functionality of a managed element of the biomechanical custodian system may be controlled using the generated control data. For example, in some embodiments, the received achievement data may include first biomechanical movement data indicative of a first biomechanical movement of the first experiencing entity after experiencing a first surgical procedure of the first condition (e.g., recovery data of the DU) and the accessed achievement data may include second biomechanical movement data indicative of a second biomechanical movement of the second experiencing entity after experiencing a second surgical procedure of the second condition (e.g., recovery data of the POI). As another example, the received achievement data may include first biomechanical movement data indicative of a first biomechanical movement of the first experiencing entity after starting a first drug treatment of the first condition and the accessed achievement data may include second biomechanical movement data indicative of a second biomechanical movement of the second experiencing entity after starting a second drug treatment of the second condition. As yet another example, each one of the first biomechanical movement and the second biomechanical movement may include cadence. As yet another example, each one of the first biomechanical movement and the second biomechanical movement may include one of ground contact time or bounce time.

It is understood that the operations shown in process 3000 of FIG. 30 are only illustrative and that existing operations may be modified or omitted, additional operations may be added, and the order of certain operations may be altered.

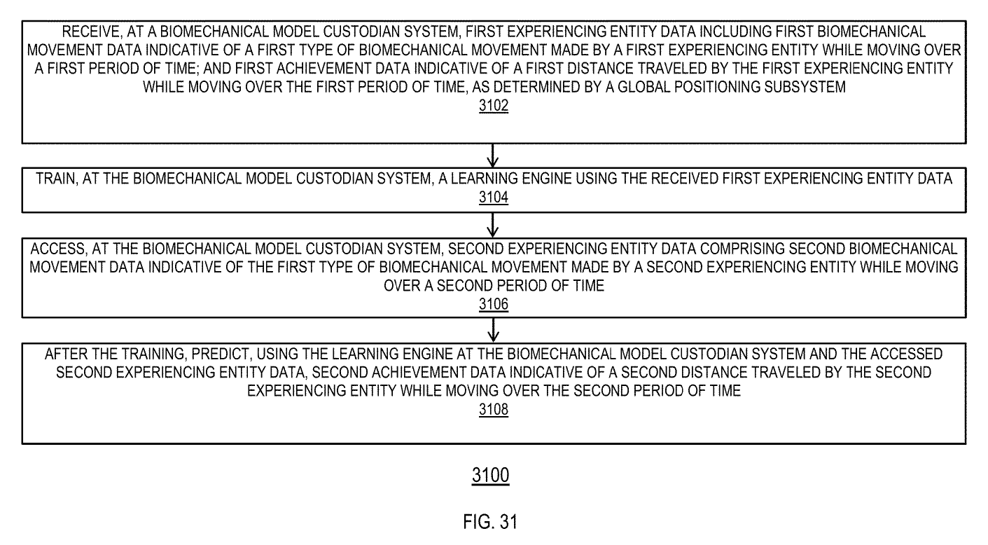

FIG. 31 is a flowchart of an illustrative process 3100 for managing biomechanical achievements using a biomechanical model custodian system that includes a global positioning subsystem (e.g., system 1). At operation 3102 of process 3100, the biomechanical model custodian system may receive first experiencing entity data that may include first biomechanical movement data indicative of a first type of biomechanical movement made by a first experiencing entity while moving over a first period of time and first achievement data indicative of a first distance traveled by the first experiencing entity while moving over the first period of time, as determined by the global positioning subsystem (e.g., biomechanical gait data of a DU walking for 10 minutes and GPS data indicating that the DU walked 1 mile in those 10 minutes). At operation 3104 of process 3100, the biomechanical model custodian system may train a learning engine using the received first experiencing entity data (e.g., system 1 may train device biomechanical model 1105a or auxiliary comfort model 1255a). At operation 3106 of process 3100, the biomechanical model custodian system may access second experiencing entity data including second biomechanical movement data indicative of the first type of biomechanical movement made by a second experiencing entity while moving over a second period of time (e.g., biomechanical gait data of a POI walking for 15 minutes). After the training of operation 3104, at operation 3108 of process 3100, the biomechanical model custodian system may predict, using the learning engine and the accessed second experiencing entity data, second achievement data indicative of a second distance traveled by the second experiencing entity while moving over the second period of time (e.g., predict that the POI walked 1.5 miles in those 15 minutes).

It is understood that the operations shown in process 3100 of FIG. 31 are only illustrative and that existing operations may be modified or omitted, additional operations may be added, and the order of certain operations may be altered.

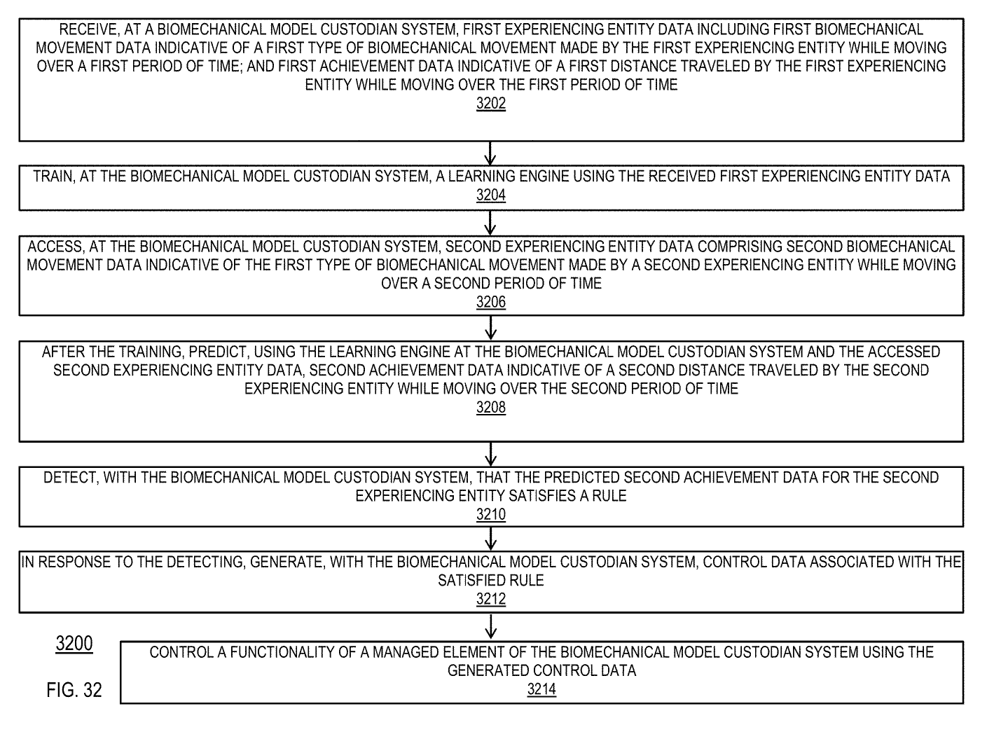

FIG. 32 is a flowchart of an illustrative process 3200 for managing biomechanical achievements using a biomechanical model custodian system (e.g., system 1). At operation 3202 of process 3200, the biomechanical model custodian system may receive first biomechanical movement data indicative of a first type of biomechanical movement made by the first experiencing entity while moving over a first period of time and first achievement data indicative of a first distance traveled by the first experiencing entity while moving over the first period of time (e.g., biomechanical gait data of a DU walking for 10 minutes and data (e.g., GPS data or user entered data) indicating that the DU walked 1 mile in those 10 minutes). At operation 3204 of process 3200, the biomechanical model custodian system may train a learning engine using the received first experiencing entity data (e.g., system 1 may train device biomechanical model 1105a or auxiliary comfort model 1255a). At operation 3206 of process 3200, the biomechanical model custodian system may access second experiencing entity data including second biomechanical movement data indicative of the first type of biomechanical movement made by a second experiencing entity while moving over a second period of time (e.g., biomechanical gait data of a POI walking for 15 minutes). After the training of operation 3204, at operation 3208 of process 3200, the biomechanical model custodian system may predict, using the learning engine and the accessed second experiencing entity data, second achievement data indicative of a second distance traveled by the second experiencing entity while moving over the second period of time (e.g., predict that the POI walked 1.5 miles in those 15 minutes). At operation 3210 of process 3200, the biomechanical model custodian system may detect that the predicted second achievement data for the second experiencing entity satisfies a rule. In response to the detecting of operation 3210, at operation 3212 of process 3200, the biomechanical model custodian system may generate control data associated with the satisfied rule. At operation 3214 of process 3200, a functionality of a managed element of the biomechanical model custodian system may be controlled using the generated control data.

It is understood that the operations shown in process 3200 of FIG. 32 are only illustrative and that existing operations may be modified or omitted, additional operations may be added, and the order of certain operations may be altered.

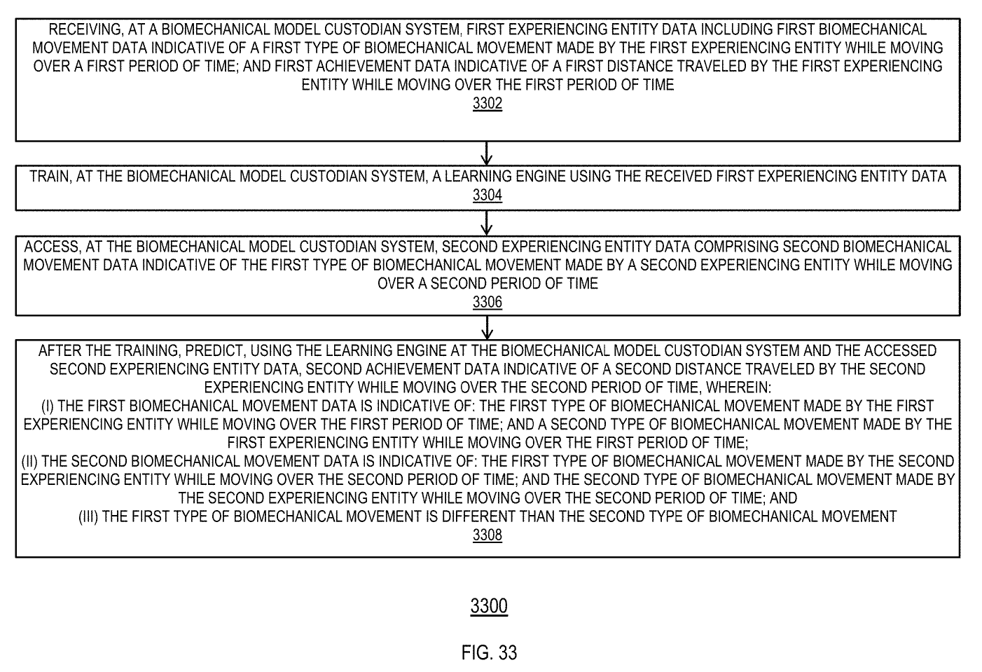

FIG. 33 is a flowchart of an illustrative process 3300 for managing biomechanical achievements using a biomechanical model custodian system (e.g., system 1). At operation 3302 of process 3200, the biomechanical model custodian system may receive first biomechanical movement data indicative of a first type of biomechanical movement made by the first experiencing entity while moving over a first period of time and first achievement data indicative of a first distance traveled by the first experiencing entity while moving over the first period of time (e.g., biomechanical gait data of a DU walking for 10 minutes and data (e.g., GPS data or user entered data) indicating that the DU walked 1 mile in those 10 minutes). At operation 3304 of process 3300, the biomechanical model custodian system may train a learning engine using the received first experiencing entity data (e.g., system 1 may train device biomechanical model 1105a or auxiliary comfort model 1255a). At operation 3306 of process 3300, the biomechanical model custodian system may access second experiencing entity data including second biomechanical movement data indicative of the first type of biomechanical movement made by a second experiencing entity while moving over a second period of time (e.g., biomechanical gait data of a POI walking for 15 minutes). After the training of operation 3304, at operation 3308 of process 3300, the biomechanical model custodian system may predict, using the learning engine and the accessed second experiencing entity data, second achievement data indicative of a second distance traveled by the second experiencing entity while moving over the second period of time (e.g., predict that the POI walked 1.5 miles in those 15 minutes), wherein the first biomechanical movement data is indicative of the first type of biomechanical movement made by the first experiencing entity while moving over the first period of time and a second type of biomechanical movement made by the first experiencing entity while moving over the first period of time, the second biomechanical movement data is indicative of the first type of biomechanical movement made by the second experiencing entity while moving over the second period of time and the second type of biomechanical movement made by the second experiencing entity while moving over the second period of time, and the first type of biomechanical movement is different than the second type of biomechanical movement (e.g., the biomechanical gait data of each one of the DU and POI may include cadence data and pelvic tilt data).

It is understood that the operations shown in process 3300 of FIG. 33 are only illustrative and that existing operations may be modified or omitted, additional operations may be added, and the order of certain operations may be altered.

It is to be understood that any suitable sensor assembly(ies) may be used to provide any suitable sensor(s) that may be configured to sense any suitable data (e.g., any suitable raw IMU data) from any suitable entities (e.g., DUs and/or POIs) that may be wearing or otherwise carrying the sensor(s) while moving in any suitable manner (e.g., walking, running, etc.). For example, any suitable suit (e.g., as described with respect to any one or more of FIGS. 1A-12) may provide any suitable sensor(s) (e.g., sensor assembly 1114 of user subsystem 1100) that, when worn, may sense any suitable movement(s) of one or more wearing users at any suitable moment(s) and/or over any suitable period(s) of time. Additionally, or alternatively, one or more distinct sensor(s) may be individually worn or carried by a user without wearing a suit (e.g., by positioning one or more sensors in a pocket, hat, watch, glove, belt, hand, etc.). The raw sensor data (e.g., data 1114′ or otherwise) that may be sensed by such sensors may be transformed into any suitable metrics (e.g., higher order metrics) and/or measurements, as any suitable sensed biomechanical movement data may be transformed into and/or may otherwise be representative of any suitable biomechanical metrics (e.g., gait metrics) of any suitable biomechanical movement(s) of the user from whom the data was sensed. Such metrics may be used to evaluate the actual performance or achievement of the user in various domains and/or to predict and evaluate future performance or achievement of the user in various domains (e.g., as biomechanical achievement state data 1222), that may determine or predict a user's sport activities, post-procedure recovery, and/or distance traveled. Such performance evaluations may then be used (e.g., with respect to any suitable rule(s) or applications) to control (e.g., as data 1224) any suitable functionality of any suitable system (e.g., any suitable managed element 1290). For example, any suitable suit (e.g., as described with respect to any one or more of FIGS. 1A-12) may provide any suitable managed element(s) (e.g., output assembly 112 and/or actuator assembly 1118 of user subsystem 1100) that, when worn, may be controlled by such performance evaluations to provide any suitable assistance and/or support and/or feedback to a wearing user at any suitable moment(s) and/or over any suitable period(s) of time.

Any suitable sensor(s) of any suitable suit (e.g., of any suit as described with respect to any one or more of FIGS. 1A-12) and/or of any other suitable sensor assembly, alone and/or in any suitable combination with any suitable processing assembly(ies), may be configured to sense from any suitable user(s) any suitable data, including, but not limited to, any suitable category data and/or any suitable achievement data of operation 1304, any suitable category data of operation 1308, any suitable gait metrics of operation 2510, any suitable gait metrics of operation 2606, any suitable experiencing entity data and/or any suitable biomechanical movement data of operation 2702, any suitable experiencing entity data and/or any suitable biomechanical movement data of operation 2706, any suitable experiencing entity data and/or any suitable biomechanical movement data of operation 2802, any suitable experiencing entity data and/or any suitable biomechanical movement data of operation 2804, any suitable category data and/or any suitable achievement data of operation 2902, any suitable category data of operation 2906, any suitable category data and/or any suitable achievement data of operation 3002, any suitable category data and/or any suitable achievement data of operation 3004, any suitable experiencing entity data and/or any suitable biomechanical movement data and/or any suitable achievement data of operation 3102, any suitable experiencing entity data and/or any suitable biomechanical movement data of operation 3106, any suitable experiencing entity data and/or any suitable biomechanical movement data and/or any suitable achievement data of operation 3202, any suitable experiencing entity data and/or any suitable biomechanical movement data of operation 3206, any suitable experiencing entity data and/or any suitable biomechanical movement data and/or any suitable achievement data of operation 3302, any suitable experiencing entity data and/or any suitable biomechanical movement data of operation 3306, and/or the like. Additionally or alternatively, any suitable actuator(s) and/or any suitable output component(s) of any suitable suit (e.g., of any suit as described with respect to any one or more of FIGS. 1A-12) and/or of any other suitable actuator or output assembly, alone and/or in any suitable combination with any suitable processing assembly(ies), may be configured to be any suitable managed element of which any suitable functionality may be controlled (e.g., defined, instructed, adjusted, manipulated, etc.) using any suitable control data, including, but not limited to, any suitable control data of operation 1304, any suitable control data of operation 2714, any suitable control data of operation 2814, any suitable control data of operation 2914, any suitable control data of operation 3014, any suitable control data of operation 3214, and/or the like. For example, any suitable actuator of any suitable suit may be controlled by such control data in any suitable manner to provide any suitable assistance and/or support to a wearing user (e.g., in response to a determination that the user may need help recovering from an experienced procedure in a particular manner). Additionally or alternatively, any suitable haptic feedback component or other suitable user output interface component (e.g., display) may be controlled by such control data in any suitable manner to provide any suitable assistance and/or support to a wearing user (e.g., in response to a determination that the user's stride length or walking speed is varying or increasing to the point of putting the user at risk of a fall).

Sensed biomechanical signals of a moving user can be stored on a sensing user subsystem (e.g., user device), in a peripheral computing subsystem, and/or on a web server or cloud database (e.g., to help train models in the cloud and/or to take advantage of macro- and/or longitudinal trends (e.g., to improve patient recovery prediction, fall risk prediction, or other machine intelligence prediction models)). A web dashboard or any other suitable mechanism may be utilized by the system to automatically summarize or display or other otherwise communicate or otherwise utilize for functionality control (e.g., at operation 1314) any suitable gait and mobility data to a patient or any suitable care providers of the patient (e.g., a family member, physician, nurse, hospital, insurance institutions, physical therapist, etc.). Data can be aggregated to give hospitals or insurance institutions and/or the like a robust understanding of the status of their patient populations and the effectiveness of various treatments and surgeries. Institutions can dive even deeper and identify, for example, which surgeons are performing the best (e.g., which surgeons' procedures (e.g., surgery events) have the highest recovery rates and which ones need more improvement or which ones should be put on probation or flagged for deeper review) and/or the data can be rolled up to also be used to help identify the top performing hospitals and clinics in the nation (e.g., institutions that may produce the highest overall recovery success rates). Eventually, this data can be used to identify best practices across physicians, clinics, and hospitals to help democratize access to information, elevate the standard of patient care, and/or improve access to high quality healthcare. Additionally or alternatively, a web dashboard or any other suitable mechanism may be utilized by the system to automatically summarize or display or other otherwise communicate or otherwise utilize for functionality control any suitable gait mobility and estimated location data that can be provided to the user, family members, care providers, coaches, or other professionals who can use the data to monitor the progress and intervene if necessary. Productivity dashboards can be aggregated across workers and safety hotspots can be mapped in the home, work, or other environments. Services can be built on top of such data to provide automated monitoring and/or auto-detection of abnormalities in behavior patterns, optimize workflow and productivity, and/or the like. As users may use the products and services of the system more and more, the data can be utilized to further refine individual, regional, and/or population models that can predict the behaviors of disease change, drug treatment efficacy, or injuries in the workplace before one ever occurs.

The systems and methods and media of the embodiments can be embodied and/or implemented at least in part as a machine configured to receive a computer-readable medium storing computer-readable instructions. The instructions can be executed by computer-executable components integrated with any suitable application, applet, host, server, network, website, communication service, communication interface, hardware/firmware/software elements of a user computer or mobile device or user subsystem, wristband, smartphone, or any suitable combination thereof. Other systems and methods and media of the embodiment can be embodied and/or implemented at least in part as a machine configured to receive a computer-readable medium storing computer-readable instructions. The instructions can be executed by computer-executable components integrated with apparatuses and networks of the type described herein. The computer-readable medium can be stored on any suitable computer readable media such as RAMs, ROMs, flash memory, EEPROMs, optical devices (e.g., CD or DVD), hard drives, floppy drives, or any suitable device. The computer-executable component can be a processor but any suitable dedicated hardware device can (e.g., alternatively or additionally) execute the instructions.

The use of one or more suitable models or engines or neural networks or the like (e.g., device biomechanical model 1105a) may enable prediction or any suitable determination of an appropriate biomechanical achievement of a user for a particular condition. Such models (e.g., neural networks) running on any suitable processing units (e.g., graphical processing units (“GPUs”) that may be available to system 1) may provide significant speed and/or power-saving improvements in efficiency and accuracy with respect to prediction over other types of algorithms and human-conducted analysis of data, as such models can provide estimates in a few milliseconds or less, thereby improving the functionality of any computing device on which they may be run. Due to such efficiency and accuracy, such models enable a technical solution for enabling the generation (e.g., at operation 1312) of any suitable control data (e.g., for controlling (e.g., a(e.g., at operation 1314) any suitable functionality of any suitable output assembly of a user subsystem or any other suitable subsystem associated with a condition (e.g., for providing alerts, recommendations, safety measures, biomechanical assistance and/or support, and/or the like to a user) using any suitable real-time data (e.g., data made available to the models) that may not be possible without the use of such models, as such models may increase performance of their computing device(s) by requiring less memory and/or less power, providing faster response times, and/or increased accuracy and/or reliability). Due to the condensed time frame and/or the time within which a decision with respect to condition data ought to be made to provide a desirable user experience, such models offer the unique ability to provide accurate determinations with the speed necessary to enable user biomechanical achievement.

Moreover, one, some, or all of the processes described with respect to FIGS. 1A-33 may each be implemented by software, but may also be implemented in hardware, firmware, or any combination of software, hardware, and firmware. They each may also be embodied as machine- or computer-readable code recorded on a machine- or computer-readable medium. The computer-readable medium may be any data storage device that can store data or instructions which can thereafter be read by a computer system. Examples of such a non-transitory computer-readable medium (e.g., memory assembly 1104 of FIG. 11) may include, but are not limited to, read-only memory, random-access memory, flash memory, CD-ROMs, DVDs, magnetic tape, removable memory cards, optical data storage devices, and the like. The computer-readable medium can also be distributed over network-coupled computer systems so that the computer-readable code is stored and executed in a distributed fashion. For example, the computer-readable medium may be communicated from one electronic device to another electronic device using any suitable communications protocol (e.g., the computer-readable medium may be communicated to user subsystem 1100 via any suitable communications assembly 1106 (e.g., as at least a portion of application 1103)). Such a transitory computer-readable medium may embody computer-readable code, instructions, data structures, program modules, or other data in a modulated data signal, such as a carrier wave or other transport mechanism, and may include any information delivery media. A modulated data signal may be a signal that has one or more of its characteristics set or changed in such a manner as to encode information in the signal.

It is to be understood that any or each module of biomechanical management system 1201 may be provided as a software construct, firmware construct, one or more hardware components, or a combination thereof. For example, any or each module of biomechanical management system 1201 may be described in the general context of computer-executable instructions, such as program modules, that may be executed by one or more computers or other devices. Generally, a program module may include one or more routines, programs, objects, components, and/or data structures that may perform one or more particular tasks or that may implement one or more particular abstract data types. It is also to be understood that the number, configuration, functionality, and interconnection of the modules of biomechanical management system 1201 are only illustrative, and that the number, configuration, functionality, and interconnection of existing modules may be modified or omitted, additional modules may be added, and the interconnection of certain modules may be altered.

At least a portion of one or more of the modules of biomechanical management system 1201 may be stored in or otherwise accessible to subsystem 1100 in any suitable manner (e.g., in memory assembly 1104 of subsystem 1100 (e.g., as at least a portion of application 1103)). Any or each module of biomechanical management system 1201 may be implemented using any suitable technologies (e.g., as one or more integrated circuit devices), and different modules may or may not be identical in structure, capabilities, and operation. Any or all of the modules or other components of biomechanical management system 1201 may be mounted on an expansion card, mounted directly on a system motherboard, or integrated into a system chipset component (e.g., into a “north bridge” chip).

Any or each module of biomechanical management system 1201 may be a dedicated system implemented using one or more expansion cards adapted for various bus standards. For example, all of the modules may be mounted on different interconnected expansion cards or all of the modules may be mounted on one expansion card. With respect to biomechanical management system 1201, by way of example only, the modules of biomechanical management system 1201 may interface with a motherboard or processor assembly 1102 of subsystem 1100 through an expansion slot (e.g., a peripheral component interconnect (“PCI”) slot or a PCI express slot). Alternatively, biomechanical management system 1201 need not be removable but may include one or more dedicated modules that may include memory (e.g., RAM) dedicated to the utilization of the module. In other embodiments, biomechanical management system 1201 may be at least partially integrated into subsystem 1100. For example, a module of biomechanical management system 1201 may utilize a portion of device memory assembly 1104 of subsystem 1100. Any or each module of biomechanical management system 1201 may include its own processing circuitry and/or memory. Alternatively, any or each module of biomechanical management system 1201 may share processing circuitry and/or memory with any other module of biomechanical management system 1201 and/or processor assembly 1102 and/or memory assembly 1104 of subsystem 1100.

In some embodiments, a powered assistive exosuit intended primarily for assistive functions can also be adapted to perform exosuit functions. In one embodiment, an assistive exosuit similar to the embodiments described in U.S. Publication No. 2018/0056104, that is used for assistive functions may be adapted to perform exosuit functions. Embodiments of such an assistive exosuit may include FLAs approximating muscle groups, such as hip flexors, gluteal/hip extensors, spinal extensors, and/or abdominal muscles. In the assistive modes of these exosuits, these FLAs may provide assistance for activities such as moving between standing and seated positions, walking, and postural stability. Actuation of specific FLAs within such an exosuit system may also provide stretching assistance. Typically, activation of one or more FLAs approximating a muscle group can stretch the antagonist muscles. For example, activation of one or more FLAs approximating the abdominal muscles might stretch the spinal extensors, or activation of one or more FLAs approximating gluteal/hip extensor muscles can stretch the hip flexors. The exosuit may be adapted to detect when the wearer is ready to initiate a stretch and perform an automated stretching regimen; or the wearer may indicate to the suit to initiate a stretching regimen.

It can be appreciated that assistive exosuits may have multiple applications. Assistive exosuits may be prescribed for medical applications. These may include therapeutic applications, such as assistance with exercise or stretching regimens for rehabilitation, disease mitigation, or other therapeutic purposes. Mobility-assistance devices such as wheelchairs, walkers, crutches, and scooters are often prescribed for individuals with mobility impairments. Likewise, an assistive exosuit may be prescribed for mobility assistance for patients with mobility impairments. Compared with mobility assistance devices, such as wheelchairs, walkers, crutches, and scooters, an assistive exosuit may be less bulky, more visually appealing, and conform with activities of daily living, such as riding in vehicles, attending community or social functions, using the toilet, and common household activities.

An assistive exosuit may additionally function as primary apparel, fashion items, or accessories. The exosuit may be stylized for desired visual appearance. The stylized design may reinforce visual perception of the assistance that the exosuit is intended to provide. For example, an assistive exosuit intended to assist with torso and upper body activities may present a visual appearance of a muscular torso and upper body. Alternatively, the stylized design may be intended to mask or camouflage the functionality of the assistive exosuit through design of the base layer, electro/mechanical integration, and/or other design factors.

Similarly to assistive exosuits intended for medically prescribed mobility assistance, assistive exosuits may be developed and utilized for non-medical mobility assistance, performance enhancement, and/or support. For many, independent aging is associated with greater quality of life, however activities may become more limited with time due to normal aging processes. An assistive exosuit may enable aging individuals living independently to electively enhance their abilities and activities. For example, gait or walking assistance could enable individuals to maintain routines such as social walking or golf. Additionally or alternatively, any suitable gait biomechanical markers may be sensed or otherwise predicted for a user in order to selectively generate control signals in certain situations for adjusting the functionality of one or more managed elements in any suitable manner (e.g., to provide warnings or instructions to the user and/or a suitable caretaker with respect to a planned or previously carried out event (e.g., surgery or physical therapy procedure) and/or to provide actuator assistance and support to the user and/or to track the location of the user without GPS data, and/or the like). Postural assistance may render social situations more comfortable, with less fatigue. Assistance with transitioning between seated and standing positions may reduce fatigue, increase confidence, and reduce the risk of falls. These types of assistance, while not explicitly medical in nature, may enable more fulfilling, independent living during aging processes.

Athletic applications for an assistive exosuit are also envisioned. In one example, an exosuit may be optimized to assist with a particular activity, such as cycling. In the cycling example, FLAs approximating gluteal or hip extensor muscles may be integrated into bicycle clothing, providing assistance with pedaling. The assistance could be varied based on terrain, fatigue level or strength of the wearer, or other factors. The assistance provided may enable increased performance, injury avoidance, or maintenance of performance in the case of injury or aging. It can be appreciated that assistive exosuits could be optimized to assist with the demands of other sports such as running, jumping, swimming, skiing, or other activities. An athletic assistive exosuit may also be optimized for training in a particular sport or activity. Assistive exosuits may guide the wearer in proper form or technique, such as a golf swing, running stride, skiing form, swimming stroke, or other components of sports or activities. Assistive exosuits may also provide resistance for strength or endurance training. The provided resistance may be according to a regimen, such as high intensity intervals.

Assistive exosuit systems as described herein may also be used in gaming applications. Motions of the wearer, detected by the suit, may be incorporated as a game controller system. For example, the suit may sense wearer's motions that simulate running, jumping, throwing, dancing, fighting, or other motions appropriate to a particular game. The suit may provide haptic feedback to the wearer, including resistance or assistance with the motions performed or other haptic feedback to the wearer.

Assistive exosuits as described herein may be used for military or first responder applications. Military and first responder personnel are often to be required to perform arduous work where safety or even life may be at stake. An assistive exosuit may provide additional strength or endurance as required for these occupations. An assistive exosuit may connect to one or more communication networks to provide communication services for the wearer, as well as remote monitoring of the suit or wearer.

Assistive exosuits as described herein may be used for industrial or occupational safety applications. Exosuits may provide more strength or endurance for specific physical tasks such as lifting or carrying or repetitive tasks such as assembly line work. By providing physical assistance, assistive exosuits may also help avoid or prevent occupational injury due overexertion or repetitive stress.

Assistive exosuits as described herein may also be configured as home accessories. Home accessory assistive exosuits may assist with household tasks such as cleaning or yard work, or may be used for recreational or exercise purposes. The communication capabilities of an assistive exosuit may connect to a home network for communication, entertainment or safety monitoring purposes.

It is to be understood that the disclosed subject matter is not limited in its application to the details of construction and to the arrangements of the components set forth in this description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art can appreciate that the conception, upon which this disclosure is based, may readily be utilized as a basis for the designing of other structures, systems, methods, and media for carrying out the several purposes of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

1.-40. (canceled)

41. A method for managing biomechanical achievements using a biomechanical model custodian system comprising a global positioning subsystem, the method comprising:
receiving, at the biomechanical model custodian system, first experiencing entity data comprising:
first biomechanical movement data indicative of a first type of biomechanical movement made by a first experiencing entity while moving over a first period of time; and
first achievement data indicative of a first distance traveled by the first experiencing entity while moving over the first period of time, as determined by the global positioning subsystem;
training, at the biomechanical model custodian system, a learning engine using the received first experiencing entity data;
accessing, at the biomechanical model custodian system, second experiencing entity data comprising second biomechanical movement data indicative of the first type of biomechanical movement made by a second experiencing entity while moving over a second period of time; and
after the training, predicting, using the learning engine at the biomechanical model custodian system and the accessed second experiencing entity data, second achievement data indicative of a second distance traveled by the second experiencing entity while moving over the second period of time.

42. The method of claim 41, wherein the first experiencing entity is the second experiencing entity.

43. The method of claim 41, wherein the first experiencing entity is different than the second experiencing entity.

44. The method of claim 41, wherein the first type of biomechanical movement comprises cadence.

45. The method of claim 41, wherein the first type of biomechanical movement comprises vertical displacement of the pelvis.

46. The method of claim 41, wherein the first type of biomechanical movement comprises pelvic transverse rotation.

47. The method of claim 41, wherein the first type of biomechanical movement comprises pelvic tilt.

48. The method of claim 41, wherein the first type of biomechanical movement comprises pelvic drop.

49. The method of claim 41, wherein:
the first biomechanical movement data is indicative of:
the first type of biomechanical movement made by the first experiencing entity while moving over the first period of time; and
a second type of biomechanical movement made by the first experiencing entity while moving over the first period of time;
the second biomechanical movement data is indicative of:
the first type of biomechanical movement made by the second experiencing entity while moving over the second period of time; and
the second type of biomechanical movement made by the second experiencing entity while moving over the second period of time; and
the first type of biomechanical movement is different than the second type of biomechanical movement.

50. The method of claim 49, wherein:
the first type of biomechanical movement comprises one of:
cadence;
vertical displacement of the pelvis;
pelvic transverse rotation;
pelvic tilt; or
pelvic drop; and
the second type of biomechanical movement comprises another one of:
cadence;
vertical displacement of the pelvis;
pelvic transverse rotation;
pelvic tilt; or
pelvic drop.

51. The method of claim 41, wherein the learning engine is a regression model.

52. The method of claim 41, further comprising:
detecting, with the biomechanical model custodian system, that the predicted second achievement data for the second experiencing entity satisfies a rule;
in response to the detecting, generating, with the biomechanical model custodian system, control data associated with the satisfied rule; and
controlling a functionality of a managed element of the biomechanical model custodian system using the generated control data.

53. The method of claim 52, wherein the rule requires that the predicted second achievement data comprises a particular variance in distance traveled by the moving second experiencing entity between a first subperiod of the second period of time and a second subperiod of the second period of time.

54. The method of claim 41, wherein:
the second experiencing entity data further comprises direction movement data indicative of at least one direction traveled by the second experiencing entity while moving over the second period of time, as determined by a magnetometer; and
the method further comprises determining, with the biomechanical model custodian system, using the predicted second achievement data and the direction movement data, a navigational pathway traversed by the second experiencing entity through space over the second period of time.

55. The method of claim 54, further comprising:
detecting, with the biomechanical model custodian system, that the determined navigational pathway satisfies a rule;
in response to the detecting, generating, with the biomechanical model custodian system, control data associated with the satisfied rule; and
controlling a functionality of a managed element of the biomechanical model custodian system using the generated control data.

56. A method for managing biomechanical achievements using a biomechanical model custodian system, the method comprising:
receiving, at the biomechanical model custodian system, first experiencing entity data comprising:
first biomechanical movement data indicative of a first type of biomechanical movement made by the first experiencing entity while moving over a first period of time; and
first achievement data indicative of a first distance traveled by the first experiencing entity while moving over the first period of time;
training, at the biomechanical model custodian system, a learning engine using the received first experiencing entity data;
accessing, at the biomechanical model custodian system, second experiencing entity data comprising second biomechanical movement data indicative of the first type of biomechanical movement made by a second experiencing entity while moving over a second period of time;
after the training, predicting, using the learning engine at the biomechanical model custodian system and the accessed second experiencing entity data, second achievement data indicative of a second distance traveled by the second experiencing entity while moving over the second period of time;
detecting, with the biomechanical model custodian system, that the predicted second achievement data for the second experiencing entity satisfies a rule;
in response to the detecting, generating, with the biomechanical model custodian system, control data associated with the satisfied rule; and
controlling a functionality of a managed element of the biomechanical model custodian system using the generated control data.

57. The method of claim 56, wherein the rule requires that the predicted second achievement data comprises a particular variance in distance traveled by the moving second experiencing entity between a first subperiod of the second period of time and a second subperiod of the second period of time.

58. The method of claim 56, wherein:
the second experiencing entity data further comprises direction movement data indicative of at least one direction traveled by the second experiencing entity while moving over the second period of time; and
the method further comprises determining, with the biomechanical model custodian system, using the predicted second achievement data and the direction movement data, a navigational pathway traversed by the second experiencing entity through space over the second period of time.

59. The method of claim 58, wherein the detecting comprises detecting that the determined navigational pathway satisfies the rule.

60. A method for managing biomechanical achievements using a biomechanical model custodian system, the method comprising:
receiving, at the biomechanical model custodian system, first experiencing entity data comprising:
first biomechanical movement data indicative of a first type of biomechanical movement made by the first experiencing entity while moving over a first period of time; and
first achievement data indicative of a first distance traveled by the first experiencing entity while moving over the first period of time;
training, at the biomechanical model custodian system, a learning engine using the received first experiencing entity data;
accessing, at the biomechanical model custodian system, second experiencing entity data comprising second biomechanical movement data indicative of the first type of biomechanical movement made by a second experiencing entity while moving over a second period of time; and
after the training, predicting, using the learning engine at the biomechanical model custodian system and the accessed second experiencing entity data, second achievement data indicative of a second distance traveled by the second experiencing entity while moving over the second period of time, wherein:
the first biomechanical movement data is indicative of:
the first type of biomechanical movement made by the first experiencing entity while moving over the first period of time; and
a second type of biomechanical movement made by the first experiencing entity while moving over the first period of time;
the second biomechanical movement data is indicative of:
the first type of biomechanical movement made by the second experiencing entity while moving over the second period of time; and
the second type of biomechanical movement made by the second experiencing entity while moving over the second period of time; and
the first type of biomechanical movement is different than the second type of biomechanical movement.
